(19) United States
(12) Patent Application Publication
(10) Pub. No.: US 2010/0143477 A1
(43) Pub. Date: Jun. 10, 2010

(54) Methods and Compositions for Stimulating the Proliferation or Differentiation of Stem Cells with Substance Por an Analog Thereof

(75) Inventors: Hal N. Siegel, Paradise Valley, AZ (US); Kasey L. Benson, Scottsdale, AZ (US)

Correspondence Address: THE LUTHER LAW FIRM, PLC, 10575 N 114TH STREET, SUITE 103, SCOTTSDALE, AZ, 85259, US

(73) Assignee: Immuneregen Biosciences, Inc., Scottsdale, AZ (US)

(21) Appl. No.: 12/670,999

(22) PCT Filed: Jul. 24, 2008
(86) PCT No.: PCT/US08/08985

§ 371 Date: Jan. 27, 2010

Related U.S. Application Data
(60) Provisional application No. 61/039,860, filed on Mar. 27, 2008.
(60) Provisional application No. 60/952,691, filed on Jul. 30, 2007.
(60) Provisional application No. 61/024,354, filed on Jan. 29, 2008.
(60) Provisional application No. 60/979,769, filed on Oct. 12, 2007.
(60) Provisional application No. 61/038,871, filed on Mar. 24, 2008.
(60) Provisional application No. 60/983,012, filed on Oct. 26, 2007.
(60) Provisional application No. 60/997,314, filed on Oct. 2, 2007.
(60) Provisional application No. 60/952,394, filed on Jul. 27, 2007.
(60) Provisional application No. 61/039,867, filed on Mar. 27, 2008.
(60) Provisional application No. 61/039,866, filed on Mar. 27, 2008.
(60) Provisional application No. 61/047,709, filed on Apr. 24, 2008.
(60) Provisional application No. 61/039,686, filed on Mar. 26, 2008.
(60) Provisional application No. 60/965,580, filed on Aug. 20, 2007.

(51) Int. Cl. A61K 38/08; C12N 5/07; A61K 9/00; A61K 35/12; A61K 35/14; A61P 17/02

(52) U.S. Cl. 424/484; 514/15; 435/325; 424/93.7

(57) ABSTRACT

Compositions and methods are provided for stimulating cell proliferation and differentiation with substance P or a substance P analog. In one embodiment, the methods provide for stimulating or promoting stem cell differentiation by contacting a stem cell with substance P or a substance P analog. In another embodiment, the methods provide for administering to subject an effective amount of substance P or a substance P analog to treat an illness, disease or disorder.

### RELATED APPLICATION DATA

This International Patent Application filed on Jul. 24, 2008, claims the benefit under 35 U.S.C. Section 119(e) of Provisional Patent Application No. 60/952,394, filed on Jul. 27, 2007; Provisional Patent Application No. 60/952,691, filed on Jul. 30, 2007; Provisional Patent Application No. 60/965,580, filed on Aug. 20, 2007, Provisional Patent Application No. 60/997,314 filed on Oct. 2, 2007; Provisional Patent Application No. 60/979,769 filed on Oct. 12, 2007; Provisional Patent Application No. 60/983,012 filed on Oct. 26, 2007; Provisional Patent Application No. 61/024,354 filed on Jan. 29, 2008; Provisional Patent Application No. 61/038,871 filed on Mar. 24, 2008; Provisional Patent Application No. 61/039,686 filed Mar. 26, 2008; Provisional Patent Application No. 61/039,867, filed on Mar. 27, 2008; Provisional Patent Application No. 61/039,866, filed on Mar. 27, 2008; Provisional Patent Application No. 61/039,860 filed Mar. 27, 2008; and Provisional Patent Application No. 61/047,709, filed Apr. 24, 2008; and is related to co-pending U.S. Patent Application PCT/U.S. ______ filed on even date herewith (also known as Attorney Docket No. IRB-004); and, co-pending International Patent Application PCT/U.S. ______ filed on even date herewith (also known as Attorney Docket No. 12241-047-228); co-pending International Patent Application PCT/U.S. ______ filed on even date herewith (also known as Attorney Docket No. 12241-046-228) co-pending International Patent Application PCT/U.S. ______ filed on even date herewith (also known as Attorney Docket No. 12241-049-228) the entire contents of each of the foregoing incorporated by reference herein.

### FIELD OF THE INVENTION

The invention relates to the field of cell differentiation. In particular, it relates to stimulation of growth and differentiation of pluripotent cells and to stimulation of growth without differentiation of pluripotent cells.

### BACKGROUND

Stem cells have tremendous potential in treating and ameliorating diseases. Yet, despite advances made in the culturing of stem cells, bathers remain. Stem cell cultures can be relatively slow to grow and the culture media can be expensive, especially for items such as cytokines and growth factors. In addition, it would be beneficial to selectively promote differentiation to specific functional cells. Improvements are needed.

In addition, the successful integration of stem cells, like human bone marrow cells (HBMCs), into potential therapies depends on a) stem cell expansion in number without differentiation; b) differentiation into a specific cell type or collection of cell types and c) promotion of their functional integration into existing tissue.

Historically, stem cells such as HBMCs have been grown on a feeder layer of mouse cells that are irradiated but viable cells and conditioned with media derived from these cells. These methods increase the risk of zoonoses acquired from the murine feeder cells and culture medium, and have significant disadvantages in reproducibility and scalability that greatly limit their clinical potential. In addition, there is a need for replenishing the HBMCs from a human donor. Although newer HBMC cell lines have been derived on human feeder layers, this system suffers from poor reproducibility and presents limits for large-scale HBMC expansion. Improvements are needed.

### SUMMARY OF THE INVENTION

The methods and compositions relate to the field of cell proliferation or differentiation. In certain embodiments the methods and compositions provide in vitro cell proliferation or differentiation. In certain embodiments the methods and compositions provide in vivo cell proliferation or differentiation.

In certain embodiments, the compositions can comprise cell media or cell culture with one or more substance P analogs, as described herein that enhance, stimulate or induce differentiation or proliferation of cells and methods relating thereto.

In certain embodiments, the compositions can comprise pharmaceutical compositions with one or more substance P analogs that can enhance, stimulate or induce differentiation or proliferation of cells in a subject in need thereof, and methods relating thereto.

In certain embodiments, the compositions can comprise pharmaceutical compositions with one or more substance P analogs and cells to promote, enhance or induce wound healing. In a preferred embodiment, the wound healing composition can comprise a three-dimensional matrix with cells and one or more substance P analogs wherein the cells are induced or promoted to differentiate or proliferate to promote wound healing.

In one aspect, the methods provide treating or ameliorating a stem cell disorder comprising administering to a subject an effective amount of a substance P analog; wherein the substance P wherein the substance P analog is of Formula (I): Z₁-Xaa¹-Xaa²-Xaa³-Xaa⁴-Xaa⁵-Xaa⁶-Xaa⁷-Xaa⁸-Xaa⁹-Xaa¹⁰-Xaa¹¹-Z₂ (I) (SEQ ID NO:11) or a pharmaceutically acceptable salt thereof, wherein Xaa¹ is Arg, Lys, 6-N methyllysine or (6-N,6-N)dimethyllysine; Xaa² is Pro or Ala; Xaa³ is Lys, Arg, 6-N-methyllysine or (6-N,6-N)dimethyllysine; Xaa⁴ is Pro or Ala; Xaa⁵ is Gln or Asn; Xaa⁶ is Gln or Asn; Xaa⁷ is Tyr, Phe or Phe substituted with chlorine at position 2, 3 or 4; Xaa⁸ is Tyr, Phe, or Phe substituted with chlorine at position 2, 3 or 4; Xaa⁹ is Gly, Pro, Ala or N-methylglycine; Xaa¹⁰ is Leu, Val, Ile, Norleucine, Met, Met sulfoxide, Met sulfone, N-methylleucine, or N-methylvaline; Xaa¹¹ is Met, Met sulfoxide, Met sulfone, or Norleucine; Z₁ is R₂N— or RC(O)NR—; Z₂ is —C(O)NR₂ or —C(O)OR or a salt thereof; each R is independently —H, (C₁-C₆) alkyl, (C₁-C₆) alkenyl, (C₁-C₆) alkynyl, (C₅-C₂₀) aryl, (C₆-C₂₆) alkaryl, 5-20 membered heteroaryl or 6-26 membered alkheteroaryl; and each “-” between residues Xaa¹ through Xaa¹¹ independently designates an amide linkage, a substitute amide linkage or an isostere of an amide.

In one embodiment, Xaa¹ is Arg; Xaa² is Pro; Xaa³ is Lys; Xaa⁴ is Pro; Xaa⁵ is Gln; Xaa⁶ is Gln; Xaa⁷ is Tyr, Phe or Phe substituted with chlorine at position 4; Xaa⁸ is Tyr, Phe, or Phe substituted with chlorine at position 4; Xaa⁹ is Gly, Pro or N-methylglycine; Xaa¹⁰ is Leu; and Xaa¹¹ is Met, Met sulfoxide, Met sulfone or Norleucine. In another embodiment, the “-” between residues Xaa¹ through Xaa¹¹ designates —C(O)NH—; Z₁ is H₂N—; and Z₂ is —C(O)NH₂. In another embodiment, the substance P analog is: RPKPQQFFGLM (SEQ ID NO:1); RPKPQQFFGLNle (SEQ ID NO:2); RPKPQQFFPLM (SEQ ID NO:3); RPKPQQFFMeGlyLM (SEQ ID NO:4); RPKPQQFTGLM (SEQ ID NO:5); RPKPQQF(4-Cl)F(4-Cl)GLM (SEQ ID NO:6); RPKPQQFFGLM(O) (SEQ ID NO:7); RPKPQQFFMeGlyLM(O) (SEQ ID NO:8); RPKPQQFFGLM(O₂) (SEQ ID NO:9); or RPKPQQFFMeGlyLM(O₂) (SEQ ID NO:10). In yet another embodiment, the substance P analog is Z₁—RPKPQQFFMeGlyLM(O₂)—Z₂; wherein Z₁ is NH₂ and Z₂ is C(O)NH₂.

In another embodiment, the stem cell disorder is amegakaryocytosis, aplastic anemia, blackfan-diamond anemia, congenital cytopenia, congenital dyserythropoietic anemia, dyskeratosis congenital, Fanconi anemia, paroxysmal nocturnal hemoglobinuria (PNH), pure red cell aplasia, acute myelofibrosis, agnogenic myeloid metaplasia, polycythemia vera, essential thrombocythemia, beta thalassemia major, sickle cell disease, familial erythrophagocytic lymphohistiocytosis, hemophagocytosis, Langerhans' cell histiocytosis (hystiocytosis X), chronic granulomatous disease, congenital neutropenia, ataxia-telangiectasia, myelokathexis, bare lymphocyte syndrome, leukocyte adhesion deficiency, severe combined immunodeficiencies (SCID), common variable immunodeficiency, bare lymphocyte syndrome, Chediak-Higashi syndrome, Kostmann syndrome, Omenn syndrome, purine nucleoside phosphorylase deficiency, reticular dysgenesis, Wiskott-Aldrich syndrome, X-linked lymphoproliferative disorder, adrenoleukodystrophy fucosidosis, Gaucher disease, Hunter's syndrome (MPS-II), Hurler's syndrome (MPS-IH), Krabbe disease, Lesch-Nyhan syndrome, mannosidosis, Maroteaux-Lamy syndrome (MPS-VI), metachromatic leukodystrophy, mucolipidosis (I-cell disease), neuronal ceroid lipofuscinosis (Batten disease), Niemann-Pick disease, Sandhoff disease, San Filippo syndrome (MPS-III), Morquio Syndrome (MPS-IV), Sly Syndrome, Beta-Glucuronidase deficiency (MPS-VII), andrenoleukodystrophy, Scheie syndrome (MPS-IS), sly syndrome, Tay Sachs, Wolman disease, Mucopolysaccharidoses (MPS), acute biphenotypic leukemia, acute lymphocytic leukemia (ALL), acute myelogenous leukemia (AML), acute undifferentiated leukemia, adult T cell leukemia, adult T cell lymphoma, chronic lymphocytic leukemia (CLL), chronic myelogenous leukemia (CML), Hodgkin's lymphoma, juvenile chronic myelogenous leukemia (JCML), juvenile myelomonocytic leukemia (JMML), myeloid/natural killer cell precursor acute leukemia, non-Hodgkin's lymphoma, polymphocytic leukemia, acute myelofibrosis, agnogenic myeloid metaplasia (myelofibrosis), amyloidosis, chronic myelomonocytic leukemia (CMML), essential thrombocythemia, polycythemia vera, multiple myeloma, plasma cell leukemia, Waldenstrom's macroglobulinemia, cartilage-hair hypoplasia, Glanzmann thrombasthenia, amegakaryocytosis, congenital thrombocytopenia, congenital erythropoietic porphyria (Gunther disease), DiGeorge syndrome, osteopetrosis, brain tumors, Ewing sarcoma, neuroblastoma, ovarian cancer, breast cancer, neuroblastoma, renal cell carcinoma, rhabodomyosarcoma, small cell lung cancer, testicular cancer, thymoma (thymic carcinoma), chronic active Epstein barr, Evans syndrome, multiple sclerosis, rheumatoid arthritis, systemic lupus erythematosus, thymic dysplasia, Chediak-Higashi syndrome, chronic granulomatous disease, neutrophil actin deficiency, reticular dysgenesis, deafness, loss of hearing, diabetes, heart disease, liver disease, muscular dystrophy, Parkinson's disease, spinal cord injury or stroke.

In another embodiment, leukocytes, lymphocytes, neutrophils, band cells, monocytes, granulocytes, erythrocytes, eosinophils, basophils or platelets are increased in the subject. In one embodiment, the lymphocytes are T lymphocytes or B lymphocytes. In another embodiment, administration of the substance P analog results in increased differentiation of high proliferative potential-stem and progenitor cells (HPP-SP cells), colony forming cells-granulocyte, erythroid, macrophage, megakaryocyte cells, (CFC-GEMM cells), granulocyte-macrophage-colony forming cells (GM-CFC), megakaryocyte-colony forming cells (Mk-CFC), T-lymphocyte-colony forming cells (T-CFC), B-lymphocyte-colony forming cells (B-CFC), colony forming unit-megakaryocyte cells (CFU-Mk cells), blast forming unit-erythroid cells (BFU-E cells), colony forming unit-erythroid cells (CFU-E cells), or colony forming unit-granulocyte/macrophage cells (CFU-GM cells). In one embodiment, the subject is human.

In another aspect, the compositions comprise a cell and a substance P analog in an amount effective to stimulate differentiation of the cell wherein the substance P analog is of Formula (I): Z₁-Xaa¹-Xaa²-Xaa³-Xaa⁴-Xaa⁵-Xaa⁶-Xaa⁷-Xaa⁸-Xaa⁹-Xaa¹⁰-Xaa¹¹-Z₂ (I) (SEQ ID NO:11) or a pharmaceutically acceptable salt thereof, wherein Xaa¹ is Arg, Lys, 6-N methyllysine or (6-N,6-N)dimethyllysine; Xaa² is Pro or Ala; Xaa³ is Lys, Arg, 6-N-methyllysine or (6-N,6-N)dimethyllysine; Xaa⁴ is Pro or Ala; Xaa⁵ is Gln or Asn; Xaa⁶ is Gln or Asn; Xaa⁷ is Tyr, Phe or Phe substituted with chlorine at position 2, 3 or 4; Xaa⁸ is Tyr, Phe, or Phe substituted with chlorine at position 2, 3 or 4; Xaa⁹ is Gly, Pro, Ala or N-methylglycine; Xaa¹⁰ is Leu, Val, Ile, Norleucine, Met, Met sulfoxide, Met sulfone, N-methylleucine, or N-methylvaline; Xaa¹¹ is Met, Met sulfoxide, Met sulfone, or Norleucine; Z₁ is R₂N— or RC(O)NR—; Z₂ is C(O)NR₂ or C(O)OR or a salt thereof; each R is independently —H, (C₁-C₆) alkyl, (C₁-C₆) alkenyl, (C₁-C₆) alkynyl, (C₅-C₂₀) aryl, (C₆-C₂₆) alkaryl, 5-20 membered heteroaryl or 6-26 membered alkheteroaryl; and each “-” between residues Xaa¹ through Xaa¹¹ independently designates an amide linkage, a substitute amide linkage or an isostere of an amide. In certain aspects, the substance P analog is not substance P.

In one embodiment, Xaa¹ is Arg; Xaa² is Pro; Xaa³ is Lys; Xaa⁴ is Pro; Xaa⁵ is Gln; Xaa⁶ is Gln; Xaa⁷ is Tyr, Phe or Phe substituted with chlorine at position 4; Xaa⁸ is Tyr, Phe, or Phe substituted with chlorine at position 4; Xaa⁹ is Gly, Pro or N-methylglycine; Xaa¹⁰ is Leu; and Xaa¹¹ is Met, Met sulfoxide, Met sulfone or Norleucine. In another embodiment, the “-” between residues Xaa¹ through Xaa¹¹ designates —C(O)NH—; Z₁ is H₂N—; and Z₂ is —C(O)NH₂. In another embodiment, the substance P analog can be RPKPQQFFGLM (SEQ ID NO:1); RPKPQQFFGLNle (SEQ ID NO:2); RPKPQQFFPLM (SEQ ID NO:3); RPKPQQFFMeGlyLM (SEQ ID NO:4); RPKPQQFTGLM (SEQ ID NO:5); RPKPQQF(4-Cl)F(4-Cl)GLM (SEQ ID NO:6); RPKPQQFFGLM(O) (SEQ ID NO:7); RPKPQQFFMeGlyLM(O) (SEQ ID NO:8); RPKPQQFFGLM(O₂) (SEQ ID NO:9); or RPKPQQFFMeGlyLM(O₂) (SEQ ID NO:10). In yet another embodiment, the substance P analog is Z₁—RPKPQQFFMeGlyLM(O₂)—Z₂; wherein Z₁ is NH₂ and Z₂ is C(O)NH₂.

In one embodiment, the cell is an undifferentiated cell. In another embodiment, the cell is a stem cell, a progenitor cell, or a partially differentiated cell. In another embodiment, the cell is a hematopoietic stem cell, lymphopoietic stem cell or myelopoietic stem cell. In another embodiment, the differentiation results in an increase in cells expressing CD15. In one embodiment, the substance P analog is administered parenterally.

In yet another aspect, the compositions provide for promoting wound healing comprising cells, a matrix, and a substance P analog wherein the substance P analog is of Formula (I): Z₁-Xaa¹-Xaa²-Xaa³-Xaa⁴-Xaa⁵-Xaa⁶-Xaa⁷-Xaa⁸-Xaa⁹-Xaa¹⁰-Xaa¹¹-Z₂ (I) or a pharmaceutically acceptable salt thereof, wherein Xaa¹ is Arg, Lys, 6-N methyllysine or (6-N,6-N)dimethyllysine; Xaa² is Pro or Ala; Xaa³ is Lys, Arg, 6-N-methyllysine or (6-N,6-N)dimethyllysine; Xaa⁴ is Pro or Ala; Xaa⁵ is Gln or Asn; Xaa⁶ is Gln or Asn; Xaa⁷ is Tyr, Phe or Phe substituted with chlorine at position 2, 3 or 4; Xaa⁸ is Tyr, Phe, or Phe substituted with chlorine at position 2, 3 or 4; Xaa⁹ is Gly, Pro, Ala or N-methylglycine; Xaa¹⁰ is Leu, Val, Ile, Norleucine, Met, Met sulfoxide, Met sulfone, N-methylleucine, or N-methylvaline; Xaa¹¹ is Met, Met sulfoxide, Met sulfone, or Norleucine; Z₁ is R₂N— or RC(O)NR—; Z₂ is —C(O)NR₂ or —C(O)OR or a salt thereof; each R is independently —H, (C₁-C₆) alkyl, (C₁-C₆) alkenyl, (C₁-C₆) alkynyl, (C₅-C₂₀) aryl, (C₆-C₂₆) alkaryl, 5-20 membered heteroaryl or 6-26 membered alkheteroaryl; and each “-” between residues Xaa¹ through Xaa¹¹ independently designates an amide linkage, a substitute amide linkage or an isostere of an amide.

In one embodiment, Xaa¹ is Arg; Xaa² is Pro; Xaa³ is Lys; Xaa⁴ is Pro; Xaa⁵ is Gln; Xaa⁶ is Gln; Xaa⁷ is Tyr, Phe or Phe substituted with chlorine at position 4; Xaa⁸ is Tyr, Phe, or Phe substituted with chlorine at position 4; Xaa⁹ is Gly, Pro or N-methylglycine; Xaa¹⁰ is Leu; and Xaa¹¹ is Met, Met sulfoxide, Met sulfone or Norleucine. In another embodiment, the “-” between residues Xaa¹ through Xaa¹¹ designates —C(O)NH—; Z₁ is H₂N—; and Z₂ is —C(O)NH₂. In yet another embodiment, the substance P analog can be RPKPQQFFGLM (SEQ ID NO:1); RPKPQQFFGLNle (SEQ ID NO:2); RPKPQQFFPLM (SEQ ID NO:3); RPKPQQFFMeGlyLM (SEQ ID NO:4); RPKPQQFTGLM (SEQ ID NO:5); RPKPQQF(4-Cl)F(4-Cl)GLM (SEQ ID NO:6); RPKPQQFFGLM(O) (SEQ ID NO:7); RPKPQQFFMeGlyLM(O) (SEQ ID NO:8); RPKPQQFFGLM(O₂) (SEQ ID NO:9); or RPKPQQFFMeGlyLM(O₂) (SEQ ID NO:10). In one embodiment, the substance P analog is Z₁—RPKPQQFFMeGlyLM(O₂)—Z₂; wherein Z₁ is NH₂ and Z₂ is C(O)NH₂.

In one embodiment, the cells are selected from the group consisting of stem cells, progenitor cells, and fibroblasts. In one embodiment, the progenitor cells are endovascular progenitor cells or endothelial progenitor cells. In another embodiment, the cells are placental cells or umbilical cord blood cells. In one embodiment, the matrix is comprised of collagen, fibrinogen, fibrin, hydrogen or amniotic membrane allograft. In another embodiment, the wound is a diabetic wound. In yet another embodiment, the wound is a decubitus ulcer.

In one aspect, the composition can comprise a cell and a substance P analog in an amount effective to stimulate proliferation of the cell, wherein the substance P analog is of Formula (I): Z₁-Xaa¹-Xaa²-Xaa³-Xaa⁴-Xaa⁵-Xaa⁶-Xaa⁷-Xaa⁸-Xaa⁹-Xaa¹⁰-Xaa¹¹-Z₂ (I) (SEQ ID NO:11) or a pharmaceutically acceptable salt thereof, wherein: Xaa¹ is Arg, Lys, 6-N methyllysine or (6-N,6-N)dimethyllysine; Xaa² is Pro or Ala; Xaa³ is Lys, Arg, 6-N-methyllysine or (6-N,6-N)dimethyllysine; Xaa⁴ is Pro or Ala; Xaa⁵ is Gln or Asn; Xaa⁶ is Gln or Asn; Xaa⁷ is Tyr, Phe or Phe substituted with chlorine at position 2, 3 or 4; Xaa⁸ is Tyr, Phe, or Phe substituted with chlorine at position 2, 3 or 4; Xaa⁹ is Gly, Pro, Ala or N-methylglycine; Xaa¹⁰ is Leu, Val, He, Norleucine, Met, Met sulfoxide, Met sulfone, N-methylleucine, or N-methylvaline; Xaa¹¹ is Met, Met sulfoxide, Met sulfone, or Norleucine; Z₁ is R₂N— or RC(O)NR—; Z₂ is —C(O)NR₂ or —C(O)OR or a salt thereof; each R is independently —H, (C₁-C₆) alkyl, (C₁-C₆) alkenyl, (C₁-C₆) alkynyl, (C₅-C₂₀) aryl, (C₆-C₂₆) alkaryl, 5-20 membered heteroaryl or 6-26 membered alkheteroaryl; and each “-” between residues Xaa¹ through Xaa¹¹ independently designates an amide linkage, a substitute amide linkage or an isostere of an amide.

In one embodiment, Xaa¹ is Arg; Xaa² is Pro; Xaa³ is Lys; Xaa⁴ is Pro; Xaa⁵ is Gln; Xaa⁶ is Gln; Xaa⁷ is Tyr, Phe or Phe substituted with chlorine at position 4; Xaa⁸ is Tyr, Phe, or Phe substituted with chlorine at position 4; Xaa⁹ is Gly, Pro or N-methylglycine; Xaa¹⁰ is Leu; and Xaa¹¹ is Met, Met sulfoxide, Met sulfone or Norleucine. In another embodiment, the “-” between residues Xaa¹ through Xaa¹¹ designates —C(O)NH—; Z₁ is H₂N—; and Z₂ is —C(O)NH₂. In yet another embodiment, the substance P analog can be RPKPQQFFGLM (SEQ ID NO:1); RPKPQQFFGLNle (SEQ ID NO:2); RPKPQQFFPLM (SEQ ID NO:3); RPKPQQFFMeGlyLM (SEQ ID NO:4); RPKPQQFTGLM (SEQ ID NO:5); RPKPQQF(4-Cl)F(4-Cl)GLM (SEQ ID NO:6); RPKPQQFFGLM(O) (SEQ ID NO:7); RPKPQQFFMeGlyLM(O) (SEQ ID NO:8); RPKPQQFFGLM(O₂) (SEQ ID NO:9); or RPKPQQFFMeGlyLM(O₂) (SEQ ID NO:10). In another embodiment, the substance P analog is Z₁—RPKPQQFFMeGlyLM(O₂)—Z₂, wherein Z₁ is NH₂ and Z₂ is C(O)NH₂.

In one embodiment, the cell is selected from the group consisting of a stem cell, a progenitor cell, a partially differentiated cell and a differentiated cell. In another embodiment, the cell is an undifferentiated cell. In another embodiment, the cell and substance P are present in an amount effective to stimulate differentiation of the cell. In another embodiment, the cell is selected from the group consisting of a stem cell, a progenitor cell, and a partially differentiated cell. In another embodiment, the cell is of hematopoietic origin.

In one aspect, the methods provide treatment for an injury, disease or disorder comprising administering to a subject substance P; wherein the substance P is in amount effective to stimulate cell proliferation to the extent necessary to treat the injury, the disease, or the disorder. In one embodiment, the injury is an injury of an internal tissue or organ. In another embodiment, the disease or the disorder is a stem cell disease or disorder. In one embodiment, the stem cell disease or disorder results from impaired cell proliferation. In another embodiment, the stem cell disease or disorder results from impaired cell differentiation. In yet another embodiment, the cell is CD34+. In one embodiment, the cell is a hematopoietic stem cell. In another embodiment, the cell is a hematopoietic progenitor cell.

In one aspect, the methods provide treatment for an injury, disease or disorder comprising administering to a subject substance P; wherein the substance P is in amount effective to stimulate cell differentiation to the extent necessary to treat the injury, the disease, or the disorder. In one embodiment, the injury is an injury of an internal tissue or organ. In another embodiment, the disease or the disorder is a stem cell disease or disorder. In one embodiment, the stem cell disease or disorder results from impaired cell proliferation. In another embodiment, the stem cell disease or disorder results from impaired cell differentiation. In yet another embodiment, the cell is CD34+. In one embodiment, the cell is a hematopoietic stem cell. In another embodiment, the cell is a hematopoietic progenitor cell. In another embodiment, the cell differentiates into a cobblestone area-forming cell. In yet another embodiment, the cell differentiates into a cell of the myeloid cell lineage. In one embodiment, the cell of the myeloid cell lineage is an erythrocyte, granulocyte, monocyte, or thrombocyte. In another embodiment, the cell differentiates into a cell of the lymphoid cell lineage. In one embodiment, the cell of the lymphoid cell lineage is a small lymphocyte.

In one aspect, the methods provide treatment for an injury, disease or disorder comprising administering to a subject a substance P analog; wherein the substance P analog is in an amount effective to stimulate cell proliferation to the extent necessary to treat the injury, the disease, or the disorder. In one embodiment, the injury is an injury of an internal tissue or organ. In another embodiment, the disease or the disorder is a stem cell disease or disorder. In one embodiment, the stem cell disease or disorder results from impaired cell proliferation. In another embodiment, the stem cell disease or disorder results from impaired cell differentiation. In yet another embodiment, the cell is CD34+. In one embodiment, the cell is a hematopoietic stem cell. In another embodiment, the cell is a hematopoietic progenitor cell.

In one aspect, the methods provide treatment for an injury, disease or disorder comprising administering to a subject a substance P analog; wherein the substance P analog is in an amount effective to stimulate cell differentiation to the extent necessary to treat the injury, the disease, or the disorder. In one embodiment, the injury is an injury of an internal tissue or organ. In another embodiment, the disease or the disorder is a stem cell disease or disorder. In one embodiment, the stem cell disease or disorder results from impaired cell proliferation. In another embodiment, the stem cell disease or disorder results from impaired cell differentiation. In yet another embodiment, the cell is CD34+. In one embodiment, the cell is a hematopoietic stem cell. In another embodiment, the cell is a hematopoietic progenitor cell. In another embodiment, the cell differentiates into a cobblestone area-forming cell. In yet another embodiment, the cell differentiates into a cell of the myeloid cell lineage. In one embodiment, the cell of the myeloid cell lineage is an erythrocyte, granulocyte, monocyte, or thrombocyte. In another embodiment, the cell differentiates into a cell of the lymphoid cell lineage. In one embodiment, the cell of the lymphoid cell lineage is a small lymphocyte.

In one aspect, the methods provide for treatment comprising upregulating an endogenous substance P pathway, wherein the upregulation is in an amount effective to stimulate cell proliferation. In one embodiment, the upregulation is accomplished by upregulation of a preprotachykinin-I gene. In another embodiment, the upregulation is accomplished by an agonist of a neurokinin I receptor.

In one aspect, the methods provide for treatment comprising upregulating endogenous substance P pathway, wherein the upregulation is in an amount effective to stimulate cell differentiation. In one embodiment, the upregulation is accomplished by upregulation of a preprotachykinin-I gene. In another embodiment, the upregulation is accomplished by an agonist of a neurokinin I receptor.

In one aspect, the methods provide for stimulating cell proliferation comprising contacting a cell with substance P; wherein the substance P is in an amount effective to stimulate cell proliferation. In one embodiment, the cell is CD34+. In another embodiment, the cell is a hematopoietic stem cell. In yet another embodiment, the cell is a hematopoietic progenitor cell.

In one aspect, the methods provide for stimulating cell proliferation comprising contacting a cell with a substance P analog; wherein the substance P analog is in an amount effective to stimulate cell proliferation. In one embodiment, the cell is CD34+. In another embodiment, the cell is a hematopoietic stem cell. In yet another embodiment, the cell is a hematopoietic progenitor cell.

In one aspect, the methods provide for stimulating cell differentiation comprising contacting a cell with substance P; wherein the substance P is in an amount effective to stimulate cell differentiation. In one embodiment, the cell is CD34+. In another embodiment, the cell is a hematopoietic stem cell. In yet another embodiment, the cell is a hematopoietic progenitor cell. In one embodiment, the cell differentiates into a cobblestone area-forming cell. In another embodiment, the cell differentiates into a cell of the myeloid cell lineage. In one another embodiment, the cell of the myeloid cell lineage is an erythrocyte, granulocyte, monocyte, or thrombocyte. In another embodiment, the cell differentiates into a cell of the lymphoid cell lineage. In one embodiment, the cell of the lymphoid cell lineage is a small lymphocyte.

In one aspect, the methods provide for stimulating cell differentiation comprising contacting a cell with a substance P analog; wherein the substance P analog is in an amount effective to stimulate cell differentiation. In one embodiment, the cell is CD34+. In another embodiment, the cell is a hematopoietic stem cell. In yet another embodiment, the cell is a hematopoietic progenitor cell. In one embodiment, the cell differentiates into a cobblestone area-forming cell. In another embodiment, the cell differentiates into a cell of the myeloid cell lineage. In one another embodiment, the cell of the myeloid cell lineage is an erythrocyte, granulocyte, monocyte, or thrombocyte. In another embodiment, the cell differentiates into a cell of the lymphoid cell lineage. In one embodiment, the cell of the lymphoid cell lineage is a small lymphocyte.

In one aspect, provided herein can be a method of culturing cells comprising contacting cells with media and substance P; wherein the substance P is in an amount effective to stimulate cell proliferation; or to the culture thereof. In one embodiment, the cell is a stem cell, a progenitor cell, a partially differentiated cell or a differentiated cell.

In one aspect, provided herein can be a method of culturing cells comprising contacting cells with media and a substance P analog; wherein the substance P analog is in an amount effective to stimulate cell proliferation; or to the culture thereof. In one embodiment, the substance P analog is present in a concentration of about 10⁻⁸M to about 10⁻¹⁸ M. In another embodiment, the cell is a stem cell, a progenitor cell, or a partially differentiated cell.

In one aspect, provided herein can be a method of culturing cells comprising contacting cells with media and substance P; wherein the substance P is in an amount effective to stimulate cell differentiation; or to the culture thereof. In one embodiment, the cell is a stem cell, a progenitor cell, a partially differentiated cell or a differentiated cell.

In one aspect, provided herein can be a method of culturing cells comprising contacting cells with media and a substance P analog; wherein the substance P analog is in an amount effective to stimulate cell differentiation; or to the culture thereof. In one embodiment, the substance P analog is present in a concentration of about 10⁻⁸M to about 10⁻¹⁸ M. In another embodiment, the cell is a stem cell, a progenitor cell, or a partially differentiated cell.

In one aspect, the compositions provide a cell culture medium comprising substance P or a substance P analog. In one embodiment, the medium further comprises a growth factor. In one embodiment, the growth factor is selected from the group consisting of: IL-2, IL-3, EPO, TPO, Flt-3, GM-CSF, G-CSF, IL-6, IL-7, SCF, and combinations of one or more of the foregoing.

In another aspect, the methods provide for increasing colony formation of CFC-GEMM, comprising contacting CFC-GEMM with an effective amount of a substance P analog, wherein an increased number of colonies are produced from the CFC-GEMM. In another embodiment, the method is for increasing colony formation of other colony types.

In yet another aspect, the compositions provide for promoting fibroblast proliferation comprising cells, a carrier, a substance P analog wherein the substance P analog is of Formula (I): Z₁-Xaa¹-Xaa²-Xaa³-Xaa⁴-Xaa⁵-Xaa⁶-Xaa⁷-Xaa⁸-Xaa⁹-Xaa¹⁰-Xaa¹¹-Z₂ (I) or a pharmaceutically acceptable salt thereof, wherein Xaa¹ is Arg, Lys, 6-N methyllysine or (6-N,6-N)dimethyllysine; Xaa² is Pro or Ala; Xaa³ is Lys, Arg, 6-N-methyllysine or (6-N,6-N)dimethyllysine; Xaa⁴ is Pro or Ala; Xaa⁵ is Gln or Asn; Xaa⁶ is Gln or Asn; Xaa⁷ is Tyr, Phe or Phe substituted with chlorine at position 2, 3 or 4; Xaa⁸ is Tyr, Phe, or Phe substituted with chlorine at position 2, 3 or 4; Xaa⁹ is Gly, Pro, Ala or N-methylglycine; Xaa¹⁰ is Leu, Val, Ile, Norleucine, Met, Met sulfoxide, Met sulfone, N-methylleucine, or N-methylvaline; Xaa¹¹ is Met, Met sulfoxide, Met sulfone, or Norleucine; Z₁ is R₂N— or RC(O)NR—; Z₂ is —C(O)NR₂ or —C(O)OR or a salt thereof; each R is independently —H, (C₁-C₆) alkyl, (C₁-C₆) alkenyl, (C₁-C₆) alkynyl, (C₅-C₂₀) aryl, (C₆-C₂₆) alkaryl, 5-20 membered heteroaryl or 6-26 membered alkheteroaryl; and each “-” between residues Xaa¹ through Xaa¹¹ independently designates an amide linkage, a substitute amide linkage or an isostere of an amide.

In one embodiment, Xaa¹ is Arg; Xaa² is Pro; Xaa³ is Lys; Xaa⁴ is Pro; Xaa⁵ is Gln; Xaa⁶ is Gln; Xaa⁷ is Tyr, Phe or Phe substituted with chlorine at position 4; Xaa⁸ is Tyr, Phe, or Phe substituted with chlorine at position 4; Xaa⁹ is Gly, Pro or N-methylglycine; Xaa¹⁰ is Leu; and Xaa¹¹ is Met, Met sulfoxide, Met sulfone or Norleucine. In another embodiment, the “-” between residues Xaa¹ through Xaa¹¹ designates —C(O)NH—; Z₁ is H₂N—; and Z₂ is —C(O)NH₂. In yet another embodiment, the substance P analog can be RPKPQQFFGLM (SEQ ID NO:1); RPKPQQFFGLNle (SEQ ID NO:2); RPKPQQFFPLM (SEQ ID NO:3); RPKPQQFFMeGlyLM (SEQ ID NO:4); RPKPQQFTGLM (SEQ ID NO:5); RPKPQQF(4-Cl)F(4-Cl)GLM (SEQ ID NO:6); RPKPQQFFGLM(O) (SEQ ID NO:7); RPKPQQFFMeGlyLM(O) (SEQ ID NO:8); RPKPQQFFGLM(O₂) (SEQ ID NO:9); or RPKPQQFFMeGlyLM(O₂) (SEQ ID NO:10). In another embodiment, the substance P analog is Z₁—RPKPQQFFMeGlyLM(O₂)—Z₂, wherein Z₁ is NH₂ and Z₂ is C(O)NH₂.

In one embodiment, the cells are stem cells, progenitor cells, fibroblasts, endothelial progenitor cells or epithelial progenitor cells. In another embodiment, the carrier is selected from the group consisting of sugar, starch, cellulose, powdered tragacanth, malt, gelatin, talc, cocoa butter, wax, oil, glycol, agar, magnesium hydroxide, aluminum hydroxide, alginic acid, pyrogen-free water, isotonic saline, Ringer's solution, ethyl alcohol, phosphate buffer solution, and lubricant.

In one aspect, the compositions provide a bandage for promoting fibroblast proliferation wherein the bandage comprises a substance P analog in an amount effective to promote fibroblast proliferation, wherein the substance P analog is of Formula (I): Z₁-Xaa¹-Xaa²-Xaa³-Xaa⁴-Xaa⁵-Xaa⁶-Xaa⁷-Xaa⁸-Xaa⁹-Xaa¹⁰-Xaa^{11-Z}₂ (I) or a pharmaceutically acceptable salt thereof, wherein Xaa¹ is Arg, Lys, 6-N methyllysine or (6-N,6-N)dimethyllysine; Xaa² is Pro or Ala; Xaa³ is Lys, Arg, 6-N-methyllysine or (6-N,6-N)dimethyllysine; Xaa⁴ is Pro or Ala; Xaa⁵ is Gln or Asn; Xaa⁶ is Gln or Asn; Xaa⁷ is Tyr, Phe or Phe substituted with chlorine at position 2, 3 or 4; Xaa⁸ is Tyr, Phe, or Phe substituted with chlorine at position 2, 3 or 4; Xaa⁹ is Gly, Pro, Ala or N-methylglycine; Xaa¹⁰ is Leu, Val, Ile, Norleucine, Met, Met sulfoxide, Met sulfone, N-methylleucine, or N-methylvaline; Xaa¹¹ is Met, Met sulfoxide, Met sulfone, or Norleucine; Z₁ is R₂N— or RC(O)NR—; Z₂ is —C(O)NR₂ or —C(O)OR or a salt thereof; each R is independently —H, (C₁-C₆) alkyl, (C₁-C₆) alkenyl, (C₁-C₆) alkynyl, (C₅-C₂₀) aryl, (C₆-C₂₆) alkaryl, 5-20 membered heteroaryl or 6-26 membered alkheteroaryl; and each “-” between residues Xaa¹ through Xaa¹¹ independently designates an amide linkage, a substitute amide linkage or an isostere of an amide.

In one embodiment, Xaa¹ is Arg; Xaa² is Pro; Xaa³ is Lys; Xaa⁴ is Pro; Xaa⁵ is Gln; Xaa⁶ is Gln; Xaa⁷ is Tyr, Phe or Phe substituted with chlorine at position 4; Xaa⁸ is Tyr, Phe, or Phe substituted with chlorine at position 4; Xaa⁹ is Gly, Pro or N-methylglycine; Xaa¹⁰ is Leu; and Xaa¹¹ is Met, Met sulfoxide, Met sulfone or Norleucine. In another embodiment, the “-” between residues Xaa¹ through Xaa¹¹ designates —C(O)NH—; Z₁ is H₂N—; and Z₂ is —C(O)NH₂. In yet another embodiment, the substance P analog can be RPKPQQFFGLM (SEQ ID NO:1); RPKPQQFFGLNle (SEQ ID NO:2); RPKPQQFFPLM (SEQ ID NO:3); RPKPQQFFMeGlyLM (SEQ ID NO:4); RPKPQQFTGLM (SEQ ID NO:5); RPKPQQF(4-Cl)F(4-Cl)GLM (SEQ ID NO:6); RPKPQQFFGLM(O) (SEQ ID NO:7); RPKPQQFFMeGlyLM(O) (SEQ ID NO:8); RPKPQQFFGLM(O₂) (SEQ ID NO:9); or RPKPQQFFMeGlyLM(O₂) (SEQ ID NO:10). In another embodiment, the substance P analog is Z₁—RPKPQQFFMeGlyLM(O₂)—Z₂, wherein Z₁ is NH₂ and Z₂ is C(O)NH₂.

In one embodiment, the bandage further comprises cells. In another embodiment, the cells are stem cells, progenitor cells, fibroblasts, endothelial progenitor cells or epithelial progenitor cells. In one embodiment, the bandage is a liquid bandage. In another embodiment, the bandage is fabricated from a material selected from the group consisting of: a gel, a matrix, a gauze, and an antimicrobial dressing.

In one aspect, the compositions provide a bandage for promoting collagen formation wherein the bandage comprises cells and a substance P analog in an amount effective to promote collagen formation wherein the substance P analog is of Formula I: Z₁-Xaa¹-Xaa²-Xaa³-Xaa⁴-Xaa⁵-Xaa⁶-Xaa⁷-Xaa⁸-Xaa⁹-Xaa¹⁰-Xaa¹¹-Z₂ (I) or a pharmaceutically acceptable salt thereof, wherein Xaa¹ is Arg, Lys, 6-N methyllysine or (6-N,6-N)dimethyllysine; Xaa² is Pro or Ala; Xaa³ is Lys, Arg, 6-N-methyllysine or (6-N,6-N)dimethyllysine; Xaa⁴ is Pro or Ala; Xaa⁵ is Gln or Asn; Xaa⁶ is Gln or Asn; Xaa⁷ is Tyr, Phe or Phe substituted with chlorine at position 2, 3 or 4; Xaa⁸ is Tyr, Phe, or Phe substituted with chlorine at position 2, 3 or 4; Xaa⁹ is Gly, Pro, Ala or N-methylglycine; Xaa¹⁰ is Leu, Val, Ile, Norleucine, Met, Met sulfoxide, Met sulfone, N-methylleucine, or N-methylvaline; Xaa¹¹ is Met, Met sulfoxide, Met sulfone, or Norleucine; Z₁ is R₂N— or RC(O)NR—; Z₂ is —C(O)NR₂ or —C(O)OR or a salt thereof; each R is independently —H, (C₁-C₆) alkyl, (C₁-C₆) alkenyl, (C₁-C₆) alkynyl, (C₅-C₂₀) aryl, (C₆-C₂₆) alkaryl, 5-20 membered heteroaryl or 6-26 membered alkheteroaryl; and each “-” between residues Xaa¹ through Xaa¹¹ independently designates an amide linkage, a substitute amide linkage or an isostere of an amide.

In one embodiment, Xaa¹ is Arg; Xaa² is Pro; Xaa³ is Lys; Xaa⁴ is Pro; Xaa⁵ is Gln; Xaa⁶ is Gln; Xaa⁷ is Tyr, Phe or Phe substituted with chlorine at position 4; Xaa⁸ is Tyr, Phe, or Phe substituted with chlorine at position 4; Xaa⁹ is Gly, Pro or N-methylglycine; Xaa¹⁰ is Leu; and Xaa¹¹ is Met, Met sulfoxide, Met sulfone or Norleucine. In another embodiment, the “-” between residues Xaa¹ through Xaa¹¹ designates —C(O)NH—; Z₁ is H₂N—; and Z₂ is —C(O)NH₂. In yet another embodiment, the substance P analog is RPKPQQFFGLM (SEQ ID NO:1); RPKPQQFFGLNle (SEQ ID NO:2); RPKPQQFFPLM (SEQ ID NO:3); RPKPQQFFMeGlyLM (SEQ ID NO:4); RPKPQQFTGLM (SEQ ID NO:5); RPKPQQF(4-Cl)F(4-Cl)GLM (SEQ ID NO:6); RPKPQQFFGLM(O) (SEQ ID NO:7); RPKPQQFFMeGlyLM(O) (SEQ ID NO:8); RPKPQQFFGLM(O₂) (SEQ ID NO:9); or RPKPQQFFMeGlyLM(O₂) (SEQ ID NO:10). In another embodiment, the substance P analog is Z₁—RPKPQQFFMeGlyLM(O₂)—Z₂, wherein Z₁ is NH₂ and Z₂ is C(O)NH₂.

In one embodiment, the cells are stem cells, progenitor cells, or fibroblasts endothelial progenitor cells or epithelial progenitor cells. In another embodiment, the bandage is a liquid bandage. In yet another embodiment, the bandage is fabricated from a material selected from the group consisting of: a gel, a matrix, a gauze, and an antimicrobial dressing.

In one aspect, the methods provide for promoting collagen formation comprising contacting a collagen-producing cell with a substance P analog in an amount effective to promote collagen production wherein the substance P analog is of Formula (I): Z₁-Xaa¹-Xaa²-Xaa³-Xaa⁴-Xaa⁵-Xaa⁶-Xaa⁷-Xaa⁸-Xaa⁹-Xaa¹⁰-Xaa¹¹-Z₂ (I) or a pharmaceutically acceptable salt thereof, wherein Xaa¹ is Arg, Lys, 6-N methyllysine or (6-N,6-N)dimethyllysine; Xaa² is Pro or Ala; Xaa³ is Lys, Arg, 6-N-methyllysine or (6-N,6-N)dimethyllysine; Xaa⁴ is Pro or Ala; Xaa⁵ is Gln or Asn; Xaa⁶ is Gln or Asn; Xaa⁷ is Tyr, Phe or Phe substituted with chlorine at position 2, 3 or 4; Xaa⁸ is Tyr, Phe, or Phe substituted with chlorine at position 2, 3 or 4; Xaa⁹ is Gly, Pro, Ala or N-methylglycine; Xaa¹⁰ is Leu, Val, Ile, Norleucine, Met, Met sulfoxide, Met sulfone, N-methylleucine, or N-methylvaline; Xaa¹¹ is Met, Met sulfoxide, Met sulfone, or Norleucine; Z₁ is R₂N— or RC(O)NR—; Z₂ is —C(O)NR₂ or —C(O)OR or a salt thereof; each R is independently —H, (C₁-C₆) alkyl, (C₁-C₆) alkenyl, (C₁-C₆) alkynyl, (C₅-C₂₀) aryl, (C₆-C₂₆) alkaryl, 5-20 membered heteroaryl or 6-26 membered alkheteroaryl; and each “-” between residues Xaa¹ through Xaa¹¹ independently designates an amide linkage, a substitute amide linkage or an isostere of an amide.

In one embodiment, Xaa¹ is Arg; Xaa² is Pro; Xaa³ is Lys; Xaa⁴ is Pro; Xaa⁵ is Gln; Xaa⁶ is Gln; Xaa⁷ is Tyr, Phe or Phe substituted with chlorine at position 4; Xaa⁸ is Tyr, Phe, or Phe substituted with chlorine at position 4; Xaa⁹ is Gly, Pro or N-methylglycine; Xaa¹⁰ is Leu; and Xaa¹¹ is Met, Met sulfoxide, Met sulfone or Norleucine. In another embodiment, the “-” between residues Xaa¹ through Xaa¹¹ designates —C(O)NH—; Z₁ is H₂N—; and Z₂ is —C(O)NH₂. In yet another embodiment, the substance P analog can be RPKPQQFFGLM (SEQ ID NO:1); RPKPQQFFGLNle (SEQ ID NO:2); RPKPQQFFPLM (SEQ ID NO:3); RPKPQQFFMeGlyLM (SEQ ID NO:4); RPKPQQFTGLM (SEQ ID NO:5); RPKPQQF(4-Cl)F(4-Cl)GLM (SEQ ID NO:6); RPKPQQFFGLM(O) (SEQ ID NO:7); RPKPQQFFMeGlyLM(O) (SEQ ID NO:8); RPKPQQFFGLM(O₂) (SEQ ID NO:9); or RPKPQQFFMeGlyLM(O₂) (SEQ ID NO:10). In another embodiment, the substance P analog is Z₁—RPKPQQFFMeGlyLM(O₂)—Z₂, wherein Z₁ is NH₂ and Z₂ is C(O)NH₂. In yet another embodiment, the collagen-producing cell is a fibroblast.

In one aspect, provided herein are methods of promoting fibroblast proliferation comprising contacting a fibroblast cell with a substance P analog in an amount effective to promote fibroblast proliferation, wherein the substance P analog is of Formula I: Z₁-Xaa¹-Xaa²-Xaa³-Xaa⁴-Xaa⁵-Xaa⁶-Xaa⁷-Xaa⁸-Xaa⁹-Xaa¹⁰-Xaa¹¹-Z₂ (I) or a pharmaceutically acceptable salt thereof, wherein Xaa¹ is Arg, Lys, 6-N methyllysine or (6-N,6-N)dimethyllysine; Xaa² is Pro or Ala; Xaa³ is Lys, Arg, 6-N-methyllysine or (6-N,6-N)dimethyllysine; Xaa⁴ is Pro or Ala; Xaa⁵ is Gln or Asn; Xaa⁶ is Gln or Asn; Xaa⁷ is Tyr, Phe or Phe substituted with chlorine at position 2, 3 or 4; Xaa⁸ is Tyr, Phe, or Phe substituted with chlorine at position 2, 3 or 4; Xaa⁹ is Gly, Pro, Ala or N-methylglycine; Xaa¹⁰ is Leu, Val, Ile, Norleucine, Met, Met sulfoxide, Met sulfone, N-methylleucine, or N-methylvaline; Xaa¹¹ is Met, Met sulfoxide, Met sulfone, or Norleucine; Z₁ is R₂N— or RC(O)NR—; Z₂ is —C(O)NR₂ or —C(O)OR or a salt thereof; each R is independently —H, (C₁-C₆) alkyl, (C₁-C₆) alkenyl, (C₁-C₆) alkynyl, (C₅-C₂₀) aryl, (C₆-C₂₆) alkaryl, 5-20 membered heteroaryl or 6-26 membered alkheteroaryl; and each “-” between residues Xaa¹ through Xaa¹¹ independently designates an amide linkage, a substitute amide linkage or an isostere of an amide.

In one embodiment, Xaa¹ is Arg; Xaa² is Pro; Xaa³ is Lys; Xaa⁴ is Pro; Xaa⁵ is Gln; Xaa⁶ is Gln; Xaa⁷ is Tyr, Phe or Phe substituted with chlorine at position 4; Xaa⁸ is Tyr, Phe, or Phe substituted with chlorine at position 4; Xaa⁹ is Gly, Pro or N-methylglycine; Xaa¹⁰ is Leu; and Xaa¹¹ is Met, Met sulfoxide, Met sulfone or Norleucine. In another embodiment, the “-” between residues Xaa¹ through Xaa¹¹ designates —C(O)NH—; Z₁ is H₂N—; and Z₂ is —C(O)NH₂. In yet another embodiment, the substance P analog can be RPKPQQFFGLM (SEQ ID NO:1); RPKPQQFFGLNle (SEQ ID NO:2); RPKPQQFFPLM (SEQ ID NO:3); RPKPQQFFMeGlyLM (SEQ ID NO:4); RPKPQQFTGLM (SEQ ID NO:5); RPKPQQF(4-Cl)F(4-Cl)GLM (SEQ ID NO:6); RPKPQQFFGLM(O) (SEQ ID NO:7); RPKPQQFFMeGlyLM(O) (SEQ ID NO:8); RPKPQQFFGLM(O₂) (SEQ ID NO:9); or RPKPQQFFMeGlyLM(O₂) (SEQ ID NO:10). In another embodiment, the substance P analog is Z₁—RPKPQQFFMeGlyLM(O₂)—Z₂, wherein Z₁ is NH₂ and Z₂ is C(O)NH₂.

In one aspect, provided herein are methods of promoting fibroblast proliferation comprising upregulating endogenous substance P activity in an amount effective to promote fibroblast proliferation. In one embodiment, the upregulation is accomplished by upregulation of a preprotachykinin-I gene. In another embodiment, the upregulation is accomplished by an agonist of a neurokinin I receptor.

In one aspect, provided herein are methods of promoting endothelial proliferation comprising upregulating endogenous substance P activity in an amount effective to promote endothelial proliferation. In one embodiment, the upregulation is accomplished by upregulation of a preprotachykinin-I gene. In another embodiment, the upregulation is accomplished by an agonist of a neurokinin I receptor.

In one aspect, provided herein are methods of promoting epithelial proliferation comprising upregulating endogenous substance P activity in an amount effective to promote epithelial proliferation. In one embodiment, the upregulation is accomplished by upregulation of a preprotachykinin-I gene. In another embodiment, the upregulation is accomplished by an agonist of a neurokinin I receptor.

In one aspect, provided herein are methods of mobilizing endothelial progenitor cells comprising contacting an endothelial progenitor cell with a substance P analog in an amount effective to stimulate mobilization, wherein the substance P analog is of Formula I: Z₁-Xaa¹-Xaa²-Xaa³-Xaa⁴-Xaa⁵-Xaa⁶-Xaa⁷-Xaa⁸-Xaa⁹-Xaa¹⁰-Xaa¹¹-Z₂ (I) or a pharmaceutically acceptable salt thereof, wherein Xaa¹ is Arg, Lys, 6-N methyllysine or (6-N,6-N)dimethyllysine; Xaa² is Pro or Ala; Xaa³ is Lys, Arg, 6-N-methyllysine or (6-N,6-N) dimethyllysine; Xaa⁴ is Pro or Ala; Xaa⁵ is Gln or Asn; Xaa⁶ is Gln or Asn; Xaa⁷ is Tyr, Phe or Phe substituted with chlorine at position 2, 3 or 4; Xaa⁸ is Tyr, Phe, or Phe substituted with chlorine at position 2, 3 or 4; Xaa⁹ is Gly, Pro, Ala or N-methylglycine; Xaa¹⁰ is Leu, Val, Ile, Norleucine, Met, Met sulfoxide, Met sulfone, N-methylleucine, or N-methylvaline; Xaa¹¹ is Met, Met sulfoxide, Met sulfone, or Norleucine; Z₁ is R₂N— or RC(O)NR—; Z₂ is —C(O)NR₂ or —C(O)OR or a salt thereof; each R is independently —H, (C₁-C₆) alkyl, (C₁-C₆) alkenyl, (C₁-C₆) alkynyl, (C₅-C₂₀) aryl, (C₆-C₂₆) alkaryl, 5-20 membered heteroaryl or 6-26 membered alkheteroaryl; and each “-” between residues Xaa¹ through Xaa¹¹ independently designates an amide linkage, a substitute amide linkage or an isostere of an amide.

In one embodiment, Xaa¹ is Arg; Xaa² is Pro; Xaa³ is Lys; Xaa⁴ is Pro; Xaa⁵ is Gln; Xaa⁶ is Gln; Xaa⁷ is Tyr, Phe or Phe substituted with chlorine at position 4; Xaa⁸ is Tyr, Phe, or Phe substituted with chlorine at position 4; Xaa⁹ is Gly, Pro or N-methylglycine; Xaa¹⁰ is Leu; and Xaa¹¹ is Met, Met sulfoxide, Met sulfone or Norleucine. In another embodiment, the “-” between residues Xaa¹ through Xaa¹¹ designates —C(O)NH—; Z₁ is H₂N—; and Z₂ is —C(O)NH₂. In yet another embodiment, the substance P analog can be RPKPQQFFGLM (SEQ ID NO:1); RPKPQQFFGLNle (SEQ ID NO:2); RPKPQQFFPLM (SEQ ID NO:3); RPKPQQFFMeGlyLM (SEQ ID NO:4); RPKPQQFTGLM (SEQ ID NO:5); RPKPQQF(4-Cl)F(4-Cl)GLM (SEQ ID NO:6); RPKPQQFFGLM(O) (SEQ ID NO:7); RPKPQQFFMeGlyLM(O) (SEQ ID NO:8); RPKPQQFFGLM(O₂) (SEQ ID NO:9); or RPKPQQFFMeGlyLM(O₂) (SEQ ID NO: 10). In another embodiment, the substance P analog is Z₁—RPKPQQFFMeGlyLM(O₂)—Z₂, wherein Z₁ is NH₂ and Z₂ is C(O)NH₂.

In one aspect, provided herein are methods of mobilizing epithelial progenitor cells comprising contacting an epithelial progenitor cell with a substance P analog in an amount effective to stimulate mobilization, wherein the substance P analog is of Formula I: Z₁-Xaa¹-Xaa²-Xaa³-Xaa⁴-Xaa⁵-Xaa⁶-Xaa⁷-Xaa⁸-Xaa⁹-Xaa¹⁰-Xaa¹¹-Z₂ (I) or a pharmaceutically acceptable salt thereof, wherein Xaa¹ is Arg, Lys, 6-N methyllysine or (6-N,6-N)dimethyllysine; Xaa² is Pro or Ala; Xaa³ is Lys, Arg, 6-N-methyllysine or (6-N,6-N)dimethyllysine; Xaa⁴ is Pro or Ala; Xaa⁵ is Gln or Asn; Xaa⁶ is Gln or Asn; Xaa⁷ is Tyr, Phe or Phe substituted with chlorine at position 2, 3 or 4; Xaa⁸ is Tyr, Phe, or Phe substituted with chlorine at position 2, 3 or 4; Xaa⁹ is Gly, Pro, Ala or N-methylglycine; Xaa¹⁰ is Leu, Val, Ile, Norleucine, Met, Met sulfoxide, Met sulfone, N-methylleucine, or N-methylvaline; Xaa¹¹ is Met, Met sulfoxide, Met sulfone, or Norleucine; Z₁ is R₂N— or RC(O)NR—; Z₂ is —C(O)NR₂ or —C(O)OR or a salt thereof; each R is independently —H, (C₁-C₆) alkyl, (C₁-C₆) alkenyl, (C₁-C₆) alkynyl, (C₅-C₂₀) aryl, (C₆-C₂₆) alkaryl, 5-20 membered heteroaryl or 6-26 membered alkheteroaryl; and each “-” between residues Xaa¹ through Xaa¹¹ independently designates an amide linkage, a substitute amide linkage or an isostere of an amide.

In one embodiment, Xaa¹ is Arg; Xaa² is Pro; Xaa³ is Lys; Xaa⁴ is Pro; Xaa⁵ is Gln; Xaa⁶ is Gln; Xaa⁷ is Tyr, Phe or Phe substituted with chlorine at position 4; Xaa⁸ is Tyr, Phe, or Phe substituted with chlorine at position 4; Xaa⁹ is Gly, Pro or N-methylglycine; Xaa¹⁰ is Leu; and Xaa¹¹ is Met, Met sulfoxide, Met sulfone or Norleucine. In another embodiment, the “-” between residues Xaa¹ through Xaa¹¹ designates —C(O)NH—; Z₁ is H₂N—; and Z₂ is —C(O)NH₂. In yet another embodiment, the substance P analog is RPKPQQFFGLM (SEQ ID NO:1); RPKPQQFFGLNle (SEQ ID NO:2); RPKPQQFFPLM (SEQ ID NO:3); RPKPQQFFMeGlyLM (SEQ ID NO:4); RPKPQQFTGLM (SEQ ID NO:5); RPKPQQF(4-Cl)F(4-Cl)GLM (SEQ ID NO:6); RPKPQQFFGLM(O) (SEQ ID NO:7); RPKPQQFFMeGlyLM(O) (SEQ ID NO:8); RPKPQQFFGLM(O₂) (SEQ ID NO:9); or RPKPQQFFMeGlyLM(O₂) (SEQ ID NO:10). In another embodiment, the substance P analog is Z₁—RPKPQQFFMeGlyLM(O₂)—Z₂, wherein Z₁ is NH₂ and Z₂ is C(O)NH₂.

In one aspect, the methods provide for increasing circulating endothelial progenitor cells in a mammal comprising: contacting a circulating endothelial progenitor cell with a substance P analog in an amount effective to increase the number of circulating endothelial cells, and wherein the substance P analog is of Formula I: Z₁-Xaa¹-Xaa²-Xaa³-Xaa⁴-Xaa⁵-Xaa⁶-Xaa⁷-Xaa⁸-Xaa⁹-Xaa¹⁰-Xaa¹¹-Z₂ (I) or a pharmaceutically acceptable salt thereof, wherein Xaa¹ is Arg, Lys, 6-N methyllysine or (6-N,6-N)dimethyllysine; Xaa² is Pro or Ala; Xaa³ is Lys, Arg, 6-N-methyllysine or (6-N,6-N)dimethyllysine; Xaa⁴ is Pro or Ala; Xaa⁵ is Gln or Asn; Xaa⁶ is Gln or Asn; Xaa⁷ is Tyr, Phe or Phe substituted with chlorine at position 2, 3 or 4; Xaa⁸ is Tyr, Phe, or Phe substituted with chlorine at position 2, 3 or 4; Xaa⁹ is Gly, Pro, Ala or N-methylglycine; Xaa¹⁰ is Leu, Val, Ile, Norleucine, Met, Met sulfoxide, Met sulfone, N-methylleucine, or N-methylvaline; Xaa¹¹ is Met, Met sulfoxide, Met sulfone, or Norleucine; Z₁ is R₂N— or RC(O)NR—; Z₂ is —C(O)NR₂ or —C(O)OR or a salt thereof; each R is independently —H, (C₁-C₆) alkyl, (C₁-C₆) alkenyl, (C₁-C₆) alkynyl, (C₅-C₂₀) aryl, (C₆-C₂₆) alkaryl, 5-20 membered heteroaryl or 6-26 membered alkheteroaryl; and each “-” between residues Xaa¹ through Xaa¹¹ independently designates an amide linkage, a substitute amide linkage or an isostere of an amide.

In one embodiment, Xaa¹ is Arg; Xaa² is Pro; Xaa³ is Lys; Xaa⁴ is Pro; Xaa⁵ is Gln; Xaa⁶ is Gln; Xaa⁷ is Tyr, Phe or Phe substituted with chlorine at position 4; Xaa⁸ is Tyr, Phe, or Phe substituted with chlorine at position 4; Xaa⁹ is Gly, Pro or N-methylglycine; Xaa¹⁰ is Leu; and Xaa¹¹ is Met, Met sulfoxide, Met sulfone or Norleucine. In another embodiment, the “-” between residues Xaa¹ through Xaa¹¹ designates —C(O)NH—; Z₁ is H₂N—; and Z₂ is —C(O)NH₂. In yet another embodiment, the substance P analog can be RPKPQQFFGLM (SEQ ID NO:1); RPKPQQFFGLNle (SEQ ID NO:2); RPKPQQFFPLM (SEQ ID NO:3); RPKPQQFFMeGlyLM (SEQ ID NO:4); RPKPQQFTGLM (SEQ ID NO:5); RPKPQQF(4-Cl)F(4-Cl)GLM (SEQ ID NO:6); RPKPQQFFGLM(O) (SEQ ID NO:7); RPKPQQFFMeGlyLM(O) (SEQ ID NO:8); RPKPQQFFGLM(O₂) (SEQ ID NO:9); or RPKPQQFFMeGlyLM(O₂) (SEQ ID NO:10). In another embodiment, the substance P analog is Z₁—RPKPQQFFMeGlyLM(O₂)—Z₂, wherein Z₁ is NH₂ and Z₂ is C(O)NH₂.

In one aspect, the methods provide for increasing circulating epithelial progenitor cells in a mammal comprising: contacting a circulating epithelial progenitor cell with a substance P analog in an amount effective to increase the number of circulating epithelial cells, and wherein the substance P analog is of Formula I: Z₁-Xaa¹-Xaa²-Xaa³-Xaa⁴-Xaa⁵-Xaa⁶-Xaa⁷-Xaa⁸-Xaa⁹-Xaa¹⁰-Xaa¹¹-Z₂ (I) or a pharmaceutically acceptable salt thereof, wherein Xaa¹ is Arg, Lys, 6-N methyllysine or (6-N,6-N)dimethyllysine; Xaa² is Pro or Ala; Xaa³ is Lys, Arg, 6-N-methyllysine or (6-N,6-N)dimethyllysine; Xaa⁴ is Pro or Ala; Xaa⁵ is Gln or Asn; Xaa⁶ is Gln or Asn; Xaa⁷ is Tyr, Phe or Phe substituted with chlorine at position 2, 3 or 4; Xaa⁸ is Tyr, Phe, or Phe substituted with chlorine at position 2, 3 or 4; Xaa⁹ is Gly, Pro, Ala or N-methylglycine; Xaa¹⁰ is Leu, Val, Ile, Norleucine, Met, Met sulfoxide, Met sulfone, N-methylleucine, or N-methylvaline; Xaa¹¹ is Met, Met sulfoxide, Met sulfone, or Norleucine; Z₁ is R₂N— or RC(O)NR—; Z₂ is —C(O)NR₂ or —C(O)OR or a salt thereof; each R is independently —H, (C₁-C₆) alkyl, (C₁-C₆) alkenyl, (C₁-C₆) alkynyl, (C₅-C₂₀) aryl, (C₆-C₂₆) alkaryl, 5-20 membered heteroaryl or 6-26 membered alkheteroaryl; and each “-” between residues Xaa¹ through Xaa¹¹ independently designates an amide linkage, a substitute amide linkage or an isostere of an amide.

In one embodiment, Xaa¹ is Arg; Xaa² is Pro; Xaa³ is Lys; Xaa⁴ is Pro; Xaa⁵ is Gln; Xaa⁶ is Gln; Xaa⁷ is Tyr, Phe or Phe substituted with chlorine at position 4; Xaa⁸ is Tyr, Phe, or Phe substituted with chlorine at position 4; Xaa⁹ is Gly, Pro or N-methylglycine; Xaa¹⁰ is Leu; and Xaa¹¹ is Met, Met sulfoxide, Met sulfone or Norleucine. In another embodiment, the “-” between residues Xaa¹ through Xaa¹¹ designates —C(O)NH—; Z₁ is H₂N—; and Z₂ is —C(O)NH₂. In yet another embodiment, the substance P analog is RPKPQQFFGLM (SEQ ID NO:1); RPKPQQFFGLNle (SEQ ID NO:2); RPKPQQFFPLM (SEQ ID NO:3); RPKPQQFFMeGlyLM (SEQ ID NO:4); RPKPQQFTGLM (SEQ ID NO:5); RPKPQQF(4-Cl)F(4-Cl)GLM (SEQ ID NO:6); RPKPQQFFGLM(O) (SEQ ID NO:7); RPKPQQFFMeGlyLM(O) (SEQ ID NO:8); RPKPQQFFGLM(O₂) (SEQ ID NO:9); or RPKPQQFFMeGlyLM(O₂) (SEQ ID NO:10). In another embodiment, the substance P analog is Z₁—RPKPQQFFMeGlyLM(O₂)—Z₂, wherein Z₁ is NH₂ and Z₂ is C(O)NH₂.

### BRIEF DESCRIPTION OF THE FIGURES

FIG. 1 provides a colony count of BFU-E vs. concentration for Homspera® (Sar⁹-SP) and substance P(SP)

FIG. 2 provides a colony count of CFU-E vs. concentration for Homspera® (Sar⁹-SP) and substance P(SP).

FIG. 3 provides a colony count of CFU-GM vs. concentration for Homspera® (Sar⁹-SP) and substance P(SP).

### DETAILED DESCRIPTION OF THE INVENTION

### Introduction

The mammalian body consists of three types of cells: somatic cells, germ cells, and stem cells. Each of the approximately 100,000,000,000,000 (10¹⁴) cells in an adult human has its own copy, or copies, of the genome, with the only exception being certain cell types that lack nuclei in their fully differentiated state, such as red blood cells. The majority of cells, including somatic and stem cells, are diploid, meaning they have two copies of each chromosome. Germ cells, the cells that give rise to gametes, are haploid, meaning that they have one copy of each chromosome.

To understand the difference between these types of cells, a discussion of cell renewal and cell differentiation is necessary. Cell renewal occurs when a cell divides into two cells that retain the characteristics of the original cell. Cell differentiation occurs when a cell divides and at least one of the two cells produced is more specialized that the original cell. An “undifferentiated” cell can refer to a cell that has not undergone differentiation, for example but not limited to, an embryonic stem cell. An “undifferentiated” cell can also refer to a cell that is not fully differentiated, i.e. still has the ability to further differentiate.

Somatic cells include most of the cells that make up the human body, including fully differentiated cells like neurons or muscle cells. Germ cells are any in a line of cells that give rise to gametes—eggs and sperm. Stem cells, on the other hand, are undifferentiated cells that can renew themselves for long periods through cell division. Stem cells can become partially or fully differentiated under certain physiological or experimental conditions.

Stem cells are generally characterized as either embryonic stem cells or adult stem cells. Embryonic stem cells are undifferentiated cells derived from embryos. Embryonic stem cells have the ability to divide for indefinite periods and to give rise ultimately to various differentiated cells. Adult stem cells are undifferentiated cells found in differentiated tissues. Adult stem cells can renew themselves and give rise to the types of cells that make up the tissue in which that adult stem cell is located.

Stem cells can give rise to progenitor cells. While stem cells can renew themselves and differentiate, progenitor cells can only differentiate. In addition, a progenitor cell is often more limited in the type of cells it can become. The majority of progenitor cells lies dormant or possesses little activity in the tissue in which they reside. The primary role of a progenitor cell is to replace cells lost by normal attrition. Upon tissue damage or injury, progenitor cells can be activated by growth factors or cytokines, leading to increased cell division important for the repair process.

Examples of stem cells and progenitor cells include but are not limited to hematopoietic stem cells (see below for further discussion); mesenchymal stem cells, adult stem cells from the bone marrow that give rise to stromal cells, fat cells, and types of bone cells; epithelial stem cells, a type of progenitor cell, that give rise to the various types of skin cells; and muscle satellite cells, a type of progenitor cell that contribute to differentiated muscle tissue.

Hematopoietic stem cells are found in, for example but not limited to, bone marrow, placenta and umbilical cord blood. Hematopoietic stem cells give rise to cells of the lymphoid and myeloid lineages. Cells of the lymphoid lineage give rise to small lymphocyte cells: white blood cells including T cell and B cells, which are important components of the immune system. Cells of the myeloid lineage give rise to erythrocytes, granulocytes, monocytes and thrombocytes. Erythrocytes are also called red blood cells and function to carry oxygen from the lungs to organs and tissues. Granulocytes include eosinophils, neutrophils and basophils, three types of white blood cells. Monocytes are white blood cells that function in the immune system to phagocytose foreign substances. Monocytes can further differentiate to give rise to macrophages, which also play a role in phagocytosis. Thrombocytes are also called platelets and function in blood clotting and immune responses.

Stromal cells are a heterogeneous population of cells naturally present in the bone marrow. Stromal cells can include cells derived from the bone marrow, including adipocytes, preadipocytes, fibroblasts, fibroblast colony forming units (CFU-F), osteoblasts, reticular cells and macrophages.

### DEFINITIONS

In order to more clearly and concisely describe the subject matter of the present invention, the following definitions are intended to provide guidance as to the usage and scope of meaning of specific terms used in the following written description, examples and appended claims.

Use of the singular forms “a,” “an,” and “the” include plural references unless the context clearly dictates otherwise. Thus, for example, reference to “a cell” includes a plurality of cells, reference to “an analog” includes a plurality of such analogs, and the like.

The term “alkyl” refers to a saturated branched, straight chain or cyclic hydrocarbon radical. Typical allyl groups include, but are not limited to, methyl, ethyl, propyl, isopropyl, butyl, isobutyl, t-butyl, pentyl, isopentyl, hexyl, and the like. In preferred embodiments, the alkyl groups are (C₁-C₆) alkyl.

The term “alkenyl” refers to an unsaturated branched, straight chain or cyclic hydrocarbon radical having at least one carbon-carbon double bond. The radical may be in either the cis or trans conformation about the double bond(s). Typical alkenyl groups include, but are not limited to, ethenyl, propenyl, isopropenyl, butenyl, isobutenyl, tert-butenyl, pentenyl, hexenyl and the like. In preferred embodiments, the alkenyl group is (C₁-C₆) alkenyl.

The term “alkynyl” refers to an unsaturated branched, straight chain or cyclic hydrocarbon radical having at least one carbon-carbon triple bond. Typical alkynyl groups include, but are not limited to, ethynyl, propynyl, butynyl, isobutynyl, pentynyl, hexynyl and the like. In preferred embodiments, the alkynyl group is (C₁-C₆) alkynyl.

The term “aryl” refers to an unsaturated cyclic hydrocarbon radical having a conjugated π electron system. Typical aryl groups include, but are not limited to, penta-2,4-diene, phenyl, naphthyl, anthracyl, azulenyl, chrysenyl, coronenyl, fluoranthenyl, indacenyl, idenyl, ovalenyl, perylenyl, phenalenyl, phenanthrenyl, picenyl, pleiadenyl, pyrenyl, pyranthrenyl, rubicenyl, and the like. In preferred embodiments, the aryl group is (C₅-C₂₀) aryl, with (C₅-C₁₀) being particularly preferred.

The term “alkaryl” refers to a straight-chain alkyl, alkenyl or alkynyl group wherein one of the hydrogen atoms bonded to a terminal carbon is replaced with an aryl moiety. Typical alkaryl groups include, but are not limited to, benzyl, benzylidene, benzylidyne, benzenobenzyl, naphthenobenzyl and the like. In preferred embodiments, the alkaryl group is (C₆-C₂₆) alkaryl, i.e., the alkyl, alkenyl or alkynyl moiety of the alkaryl group is (C₁-C₆) and the aryl moiety is (C₅-C₂₀). In particularly preferred embodiments, the alkaryl group is (C₆-C₁₃) alkaryl, i.e., the alkyl, alkenyl or alkynyl moiety of the alkaryl group is (C₁-C₃) and the aryl moiety is (C₅-C₁₀).

The term “alkheteroaryl” refers to a straight-chain alkyl, alkenyl or alkynyl group where one of the hydrogen atoms bonded to a terminal carbon atom is replaced with a heteroaryl moiety. In preferred embodiments, the alkheteroaryl group is 6-26 membered alkheteroaryl, i.e., the alkyl, alkenyl or alkynyl moiety of the alkheteroaryl is (C₁-C₆) and the heteroaryl is a 5-20-membered heteroaryl. In particularly preferred embodiments the alkheteroaryl is 6-13 membered alkheteroaryl, i.e., the alkyl, alkenyl or alkynyl moiety is a 5-10 membered heteroaryl.

The term “cell differentiation” or “differentiation” refers to maturation or specialization of a cell. The cell that is more specialized than its previous cell can be said to have differentiated, or partially differentiated, depending upon whether the cell will continue to differentiate. Measures of cell differentiation are discussed elsewhere herein. Impaired differentiation refers to any abnormality that may occur throughout the process of differentiation, including but not limited to, the inability to express cellular markers associated with a particular differentiated cell type.

The term “cell proliferation” or “proliferation” refers to the process in which a cell divides to produce more cells. Measures of cell proliferation are discussed elsewhere herein.

The term “cobblestone area-forming cells” refers to a fraction of hematopoietic stem cells that migrate to form a non-refractile layer of cells underneath a layer of stromal feeder cells in a cobblestone area-forming cell assay.

The term “heteroaryl” refers to an aryl moiety wherein one or more carbon atoms is replaced with another atom, such as N, P, O, S, As, Se, Si, Te, etc. Typical heteroaryl groups include, but are not limited to, acridarsine, acridine, arsanthridine, arsindole, arsindoline, carbazole, β-carboline, chromene, cinnoline, furan, imidazole, indazole, indole, indolizine, isoarsindole, isoarsinoline, isobenzofuran, isochromene, isoindole, isophosphoindole, isophosphinoline, isoquinoline, isothiazole, isoxazole, naphthyridine, perimidine, phenanthridine, phenanthroline, phenazine, phosphoindole, phosphinoline, phthalazine, pteridine, purine, pyran, pyrazine, pyrazole, pyridazine, pyridine, pyrimidine, pyrrole, pyrrolizine, quinazoline, quinoline, quinolizine, quinoxaline, selenophene, tellurophene, thiophene and xanthene. In preferred embodiments, the heteroaryl group is a 5-20 membered heteroaryl, with 5-10 membered aryl being particularly preferred.

The term “liquid bandage” refers to a liquid or gel that is able to substantially increase in viscosity when placed over a wound to promote healing. According to the compositions and methods provided herein, a liquid bandage comprising stem cells or progenitor cells or combinations thereof and one or more substance P analogs in a liquid or gel that is able to increase in viscosity to a near solid form that can be placed on or over a wound to promote healing.

The term “lymphopoietic cell” refers to cells of the lymphoid lineage, including T-cells, B-cells or natural killer (NK) cells at any stage of maturity including precursor cells.

The term “pharmaceutically acceptable carrier” or “carrier” refers to a non-toxic, inert solid, semi-solid or liquid filler, excipient, diluent, encapsulating material or formulation auxiliary of any type.

The term “progenitor cell” or “progenitors” refers to a cell committed to differentiate to a specific type of cell or lineage. These cells are typically unipotent or multipotent and are generally not capable of self-renewal.

The term “stem cell” refers to undifferentiated cells that can renew themselves for long periods through cell division (e.g. “self-renewal”). Stem cells can become partially or fully differentiated under certain physiological or experimental conditions. Stem cells can be pluripotent or multipotent.

The term “stem cell disease” or a “stem cell disorder” refers to any illness, disorder or disease that can be prevented, treated or ameliorated by affecting the genotype or phenotype of a stem cell, such as but not limited to, stem cell proliferation or differentiation.

The term “substituted alkyl, alkenyl, alkynyl, aryl alkaryl, heteroaryl or alkheteroaryl” refers to an alkyl, alkenyl, alkynyl, aryl, alkaryl, heteroaryl or alkheteroaryl group in which one or more hydrogen atoms is replaced with another substituent. Preferred substituents include —OR, —SR, —NRR, —NO₂, —CN, halogen, —C(O)R, —C(O)OR and —C(O)NR, where each R is independently hydrogen, alkyl, alkenyl, alkynyl, aryl, alkaryl, heteroaryl or alkheteroaryl.

The term “undifferentiated” cell can refer to a cell that has not undergone differentiation, for example but not limited to, an embryonic stem cell. An “undifferentiated” cell can also refer to a cell that is not fully differentiated, i.e. still has the ability to further differentiate.

The term “upregulating an endogenous substance P pathway” refers to increasing the activity of any pathway in which substance P functions. For example, increasing the transcription of the preprotachykinin I gene, which encodes substance P, can upregulate an endogenous substance P pathway. Another example of upregulating a substance P pathway is activating a receptor on which substance P acts, e.g. neurokinin I. Neurokinin I can be activated, for example, by an agonist.

### Methods

The amino terminus, peptides 1-4 (RPKP) of endogenous substance P has been shown to inhibit the proliferation of both lymphoid and myeloid stem and progenitor cells (see U.S. Pat. No. 7,119,071). While native substance P (RPKPQQFFGLM (SEQ ID NO:1)) has also been shown to stimulate bone marrow progenitors in erythroid and myeloid lineages. Rameshwar, et al., 1993, Blood, 81(2) 391-398. However, applicants have surprisingly discovered that certain substance P analogs, as described herein, stimulate proliferation or differentiation of stem cells at lower concentrations (in some cases log scale less) than the native compound and substance P analogs containing the identical amino-terminus (RPKP) stimulate proliferation or differentiation of stem cells at much lower concentrations than does substance P.

In one embodiment, the substance P analog [Sar⁹,Met(O)₂¹¹]-substance P, as described herein, can be used to stimulate proliferation or differentiation of stem cells. The sequence of [Sar⁹, Met(O)₂¹¹]-substance P can also be written as RPKPQQFFMeGlyLM(O₂) (SEQ ID NO:10). [Sar⁹, Met(O)₂¹¹]-substance P is also referred to as Homspera®. In one embodiment, the concentration of a substance P analog used to stimulate proliferation or differentiation of stem cells can be about 3 orders of magnitude less (3 log scale less) than the concentration of substance P used. Provided herein are methods for stimulation, promotion or enhancement of human stem cell proliferation or differentiation.

According to the compositions and methods described herein, substance P or substance P analogs can be used to stimulate proliferation or differentiation of stem cells, progenitor cells, or any other cell that has the ability to proliferate or differentiate. In one embodiment, non-stem cells that have been re-programmed to behave as stem cells can be used with the compositions and methods described herein. For example, a differentiated cell with little or no proliferative potential can be transfected with genes encoding transcriptional regulators to become more like stem cells. See Takahashi, K. & Yamanaka, S., 2006, Cell 126:663-676.

In one embodiment, the methods provide for stimulating, promoting or enhancing proliferation or differentiation of stem cells in vitro in the presence of a substance P or a substance P analog. In some embodiments, the methods provide for selective differentiation of undifferentiated or nave cells, for example but not limited to, stem cells. The type of stem cell that is preferred can be detected by the presence or absence of cell surface markers.

In one embodiment, the methods provide for stimulating, promoting or enhancing proliferation or differentiation of stem cells in vivo by the administration of substance P or a substance P analog. In one embodiment, the one or more substance P analogs can be administered to a subject with an illness, disorder or disease that can be treated or ameliorated by increasing stem cell proliferation or differentiation.

It can be advantageous to proliferate stem cells in vitro without stimulating, enhancing or encouraging differentiation. In one embodiment the methods provide for stimulating stem cell proliferation in vitro in the presence of a substance P analog or in vivo by the administration of a substance P analog. In one embodiment, human bone marrow cells (HBMCs) are stimulated to proliferate in vitro in the presence of a substance P analog or in vivo by the administration of a substance P analog.

### Cell Type

In one embodiment, the methods provide for enhanced proliferation or differentiation of hematopoietic stem or progenitor cells. In one embodiment, the methods provide for stimulated or enhanced proliferation or differentiation of hematopoietic cells in vivo or in vitro. In one embodiment the methods provide for stimulation of hematopoietic progenitor colony formation in vitro. In one embodiment, the methods provide for enhanced proliferation or differentiation of lymphopoietic cells in vivo or in vitro.

In one embodiment, the stem cells can be lymphoid cells. In another embodiment, the stem cells can be hematopoietic cells. In one embodiment, the stem cells can be stromal cells. In one embodiment, the methods can be used to stimulate proliferation of granulocytes, macrophages, erythrocytes, lymphocytes or thrombocytes (platelets).

In one embodiment, the methods provide for stimulating or promoting mesenchymal stem cell differentiation with substance P or a substance P analog. In one embodiment, the methods provide for stimulating or promoting epithelial stem cell differentiation with substance P or a substance P analog. In one embodiment, the methods provide for stimulating or promoting muscle satellite cell differentiation with substance P or a substance P analog.

In one embodiment the cell population that can be stimulated to proliferate can be High Proliferative Potential-Stem and Progenitor Cells (HPP-SP), Colony-Forming Cell-Granulocyte, Erythroid, macrophage, Megakaryocyte (CFC-GEMM), Blast-Forming Unit-Erythroid (BFU-E), Granulocyte-Macrophage Colony Forming Cell (GM-CFC), Megakaryocyte Colony-Forming Cell (Mk-CFC), T-lymphocyte Colony-Forming Cell (T-CFC), or B-lymphocyte Colony-Forming Cell (B-CFC).

The HPP-SP population comprises stem and progenitor cells that express CD90+/CD133+/CD34+ markers. The BFU-E population is comprised of erythroid cells expressing CD38+/Glycophorin-A+ markers. The GM-CFC population comprises granulocyte-macrophage colony forming cells expressing CD38+/CD14+/CD15+ markers. The Mk-CFC population comprises megakaryocyte colony forming cells expressing CD41+/CD61+ markers. The T-CFC population comprises T-lymphocyte colony forming cells expressing CD3+/CD4+/CD8+ cell markers. The B-CFC population comprises B-lymphocyte colony forming cells expressing CD19+ markers. The CFC-GEMM population comprises granulocyte, erythroid, macrophage and megakaryocyte cells. Cells of the CFC-GEMM population display cell markers for their particular lineage as well as CD34+ and CD133+ (e.g. granulocytes will display CD38+/CD14+/CD15+ as well as CD34+ and CD133+ markers, erythroid cells will express CD38+/Glycophorin-A+ markers as well as CD34+ and CD133+ markers).

In one embodiment, the stem cells can be lymphoid cells. In another embodiment, the stem cells can be myeloid cells. In one embodiment, the stem cells can be mesenchymal stem cells. In one embodiment, the stem cells can be unipotent, pluripotent or multipotent.

In one embodiment, the methods can be used to stimulate differentiation to progenitor cells, granulocytes, macrophages, erythrocytes, lymphocytes or platelets.

In one embodiment the methods can be used to promote or stimulate differentiation of stem cells to glia, myocardium, hepatocytes, cochlear cells, osteoblasts, chondrocytes, myocyte, adipocytes, β-pancreatic islet cells, neuronal cells, connective tissue, fibroblasts, skin, cartilage or bone.

In one embodiment, provided herein are methods for promoting or stimulating proliferation of stromal, mesenchymal or placental cell differentiation comprising: administering to the animal an effective amount of a substance P analog.

In one embodiment, the mesenchymal stem cells can be promoted to differentiate to stromal cells, fat cells, and types of bone cells. In one embodiment, the epithelial stem cells (progenitor cells) can be promoted to differentiate to various types of skin cells. In one embodiment, the muscle satellite cells (progenitor cells) can be promoted to differentiate to muscle tissue. In a preferred embodiment, the methods of the invention can be used with placental cells. In a more preferred embodiment, the methods of the invention can be used with human placental cells.

### Cell Source

Stem cells useful with methods and compositions described herein can be from any source. In one embodiment, the stem cells can be obtained from bone marrow, preferably from a living human (adult stem cells). In one embodiment, the stem cells can be obtained from established tissue cultures or cell lines, for example but limited to, California Registry established with Proposition 71 or the National Institute of Health Stem Cell Registry. In one embodiment, the stem cells can be from menstrual blood. Meng et al., 2007, J. Transl. Med. 5(57): 1-10, Gargett et al., 2007, Curr. Opin. Obstet. Gynecol 19: 377-383, Gargett and Chan 2006, Minerva Ginecol. 58(6): 511-26. In one embodiment, the stem cells can be derived from deciduous teeth. Miura, M. et al., (2003) Proc. Nat. Acad. Sci. 100(10): 5807-5812. In another embodiment, stem cells can be derived from amniotic fluid. De Coppi, P. et al. (2007), Nature Biotechnology 25, 100-106. In another embodiment, the stem cells can be cochlear stem cells, hair cells or undifferentiated cells of the organ of Corti. See, Lin et al., 2007, Curr. Med. Chem. 14(27): 2937-43, Yerukhimovich et al., 2007, Dev. Neurosci. 29(3): 251-260.

In one embodiment, the stem cells can be embryonic stem cells. Embryonic stem cells can be obtained from placenta or umbilical cord blood or placental-derived adherent cells (PDAC, Celgene Corp. NJ). See, U.S. Pat. Nos. 7,045,148, 7,255,879, 7,311,905, 5,486,359, 5,004,681, 5,192,553, incorporated herein by reference in their entirety.

### Culture Conditions

Growth of stem cells in culture can be facilitated by using a medium comprising an array of cytokines and growth factors. Such cytokines and growth factors are available from a number of commercial vendors such as Biosource, Sigma-Aldrich and Apollo Cytokine Research, for example. Cytokines and growth factors used in stem cell cultures include but are not limited to erythropoietin (EPO), granulocyte macrophage-colony stimulating factor (GM-CSF), interleukin-3 (IL-3), interleukin-6 (IL-6), stem cell factor (SCF), thrombopoietin (TPO), flat 3 ligand (Flt3-L), interleukin-2 (IL-2), interleukin-7 (IL-7), brain-derived neurotrophic factor (BDNF), bone morphogenic protein-2 (BMP-2), bone morphogenic protein-7 (BMP-7), ciliary neurotrophic factor (CNTF), epidermal growth factor (EGF), acidic fibroblast growth factor (aFGF), basic fibroblast growth factor (bFGF), fibroblast growth factor-4 (FGF-4), fibroblast growth factor-5 (FGF-5), fibroblast growth factor-6 (FGF-6), fibroblast growth factor-8 (FGF-8), fibroblast growth factor-9 (FGF-9), fibroblast growth factor-10 (FGF-10), fibroblast growth factor-16 (FGF-16), fibroblast growth factor-17 (FGF-17), fibroblast growth factor-18 (FGF-18), fibroblast growth factor-19 (FGF-19), fibronectin, glial-derived neurotropic factor (GDNF), interferon-γ (IFN-γ), insulin-like growth factor-I (IGF-I), insulin-like growth factor II (IGF-II), interleukin-1β(IL-1β), interleukin-8 (IL-8), interleukin-11 (IL-11), interleukin-12 (IL-12), insulin, keratinocyte growth factor (KGF), laminin, myelin basic protein (MBP), nerve growth factor (NGF), nerve growth factor 7S (NGF 7S), neurturin, neurotrophic factor-3 (NT-3), neurotrophic factor-4 (NT-4), oncostatin M, platelet derived growth factor AA homodimer (PDGF-AA), platelet derived growth factor AB (PDGF-AB), platelet derived growth factor BB (PDGF-BB), α-synuclein, β-synuclein, transforming growth factor-α (TGF-α), transforming growth factor-β1 (TGF-β1), transforming growth factor-β2 (TGF-β2), tumor necrosis factor-α (TNF-α), vascular endothelial cell growth factor (VEGF), sonic hedgehog (SHH), retinoic acid (RA) or combinations thereof.

In another embodiment, the cells, for example, lymphopoietic cells, are cultured in a media comprised of growth factors and cytokines in the presence of one or more substance P analogs. The substance P analogs can also act to enhance or potentiate the effect of lineage-specific growth factors. The methods provided herein can increase selective differentiation of stem cells to granulocyte macrophage progenitors. In one embodiment, the selective differentiation provides for an increase of about 200% to about 250% of functional cells differentiated from stem cells over control populations.

In one embodiment, stem cells can be stimulated to differentiate in vitro in the presence of an effective amount of one or more substance P analogs and cytokines or growth factors. In the presence of substance P analogs, the concentrations of growth factors and cytokines used can be lower than would otherwise be used, also referred to as “sub-optimal” concentrations of growth factors and cytokines. The amount of growth factor or cytokine used in vitro to stimulate stem cell growth and differentiation varies according to the type of cell population as is know in the art. See, Rameshwar et al. 1993, Blood, 81(2): 391-398, Rich, and Hall, 2005, Toxicol. Sci., 87(2): 427-441, U.S. Pat. Nos. 7,354,729, 7354,730 and U.S. patent application Ser. No. 11/561,133. For example, one technique in the art is to culture a GM-CFC population in a culture with about 20 ng of Granulocyte Macrophage-Colony Stimulating Factor (GM-CSF), about 10 ng of interleukin-3 (IL-3) and about 50 ng of stem cell factor (SCF). The addition of a substance P analog allows culture of GM-CFC with reduced cytokines and growth factors (about 0.4 ng of GM-CSF, about 0.2 ng of IL-3 and about 1 ng of SCF). See, Example 3.

In one embodiment, reductions of cytokines or growth factors in the culture media can be about 20%, about 30%, about 40%, about 50%, about 60% or about 70% less than those concentrations which are effective in the absence of substance P or its analogs. In certain embodiments the methods and compositions provide for cell culture using about 20 fold to about 50 fold less than those concentrations which are effective in the absence of substance P or its analogs.

In one embodiment, the substance P analog concentration in the cell culture can be about 10⁻⁶ M, about 10⁻⁷ M, about 10⁻⁸ M, about 10⁻⁹ M, about 10⁻¹⁰ M, about 10⁻¹¹ M, about 10⁻¹² M, about 10⁻¹³ M, about 10⁻¹⁴M, about 10⁻¹⁵M, about 10⁻¹⁶M or about 10⁻¹⁷M. In a preferred embodiment, the substance P analog concentration in the cell culture can be about 10⁻⁹ M to about 10⁻¹⁴M.

### Assessment of Proliferation or Differentiation

In one embodiment, proliferation of hematopoietic stem cells can be stimulated in vitro in the presence of a substance P analog at about 50%, about 100%, about 125%, about 150%, about 175% or about 200%, or greater than when stems cells are cultured in the absence of a substance P analog. In one preferred embodiment, lymphopoietic cells can be proliferated at a rate of about 150%, about 200%, about 250%, about 300%, about 350% or greater over control populations.

Proliferation can be assessed via intracellular adenosine triphosphate (iATP) levels. Rich and Hall 2005, Tox. Sci. 87(2): 427-441 and Reems et al., 2008 Transfusion, 48:620-628. In one embodiment, the methods provide for proliferation of stems cells using a substance P analog wherein the iATP can be about 0.5 μM/well, about 1.0 μM/well or about 1.5 μM/well.

In one embodiment, the methods provide for increases in differentiation as measured by colony-forming cell assay. In certain embodiments, the methods provide for increases of about 50%, about 100%, about 150%, about 200%, about 250%, about 300% or more. In one embodiment, differentiation can be indicated by an increase in colony forming units of hematopoietic progenitor cells.

Stem cell populations can be identified by cell surface markers using any means known to those of skill in the art, including but not limited to, for example, fluorescently-labeled antibodies directed to the specific cluster of differentiation (CD) antigen. Fluorescence techniques known in the art can be used with the methods described herein. See, for example, Kusser and Randall, 2003, J. Histochem. Cytochem. 51:5-14. Other methods of detecting stem cell differentiation can be used with the methods described herein, including for example, the use of a reporter gene. Eiges et al., 2001, Curr. Biol. 11: 514-518.

In one embodiment the cell marker can be, but is not limited to, fetal liver kinase-1 (Flk1), smooth muscle cell-specific myosin heavy chain, vascular endothelial cell cadherin, bone-specific alkaline phosphatase (BAP), hydroxyapatite, osteocalcin (OC), bone morphogenetic protein receptor (BMPR), CD4, CD9, CD14, CD15, CD29, CD41, CD41A, CD59, CD73, CD90, CD105, CD8, CD34, CD34+ Sco1+ Lin− profile, CD38, CD44, CD61, c-Kit, Colony-forming unit (CFU), fibroblast colony-forming unit (CFU-F), Hoechst dye, leukocyte common antigen (CD45), lineage surface antigen (Lin), Mac-1, glycophorin-A (CD235a), 7-aminoactinomycin D (7-AAD), CD38, CD117, CD3, CD19, CD56, Muc-18 (CD146), stem cell antigen (Sca-1), Stro-1 antigen, Thy-1, collagen types II and IV, keratin, sulfated proteoglycan, adipocyte lipid-binding protein (ALBP), fatty acid transporter (FAT), adipocyte lipid-binding protein (ALBP), Y chromosome, karyotype, albumin, B-1 integrin, CD133, glial fibrillary acidic protein (GFAP), microtubule-associated protein-1 (MAP-2), myelin basic protein (MPB), nestin, neural tubulin, neurofilament (NF), neurosphere, noggin, O4, O1, synaptophysin, tau, cytokeratin 19 (CK19), glucagon, insulin, insulin-promoting factor-1 (PDX-1), pancreatic polypeptide, somatostatin, alkaline phosphatase, alpha-fetoprotein (AFP), bone morphogenetic protein-4, brachyury, cluster designation 30 (CD30), crypto (TDGF-1), GATA-4 gene, GCTM-1, genesis, germ cell nuclear factor, hepatocyte nuclear factor-5 (HNF-4), neuronal cell-adhesion molecule (N-CAM), polysialic acid-neural cell adhesion molecule (PSA-NCAM), Oct-4, Pax6, stage-specific embryonic antigen-3 (SSEA-3), stage-specific embryonic antigen-4 (SSEA-4), stem cell factor (SCF or c-kit ligand), telomerase, TRA-1-60, TRA-1-81, vimentin, MyoD, Pax&, Myogenin, MR4, myosin heavy chain, myosin light chain, CD150, CD48, ATP-binding cassette superfamily G member 2 (ABCG2), p75 Neurotrophin R (NTR), Musashi homolog 1 (MSI1), SRY (sex determining region Y)-box (SOX) family of transcription factors, Sox2, nanog, CUB domain containing protein 1 (CDCP1), pu1 transcription factor, twist 1 transcription factor, POU domain class 5 transcription factor 1 (POU5F1), REX1 transcription factor, podocalyxin, or telomerase reverse transcriptase (TERT), Otx2, Myo7a or combinations thereof.

In a preferred embodiment, the cell markers can be those that are useful as an indicia of differentiation, including but not limited to, for example, CD200, CD105, CD10, CD34, CD44, CD45, CD133, CD90, CD117, HLA-G expression or cytokeratin.

### Therapeutic and Clinical Applications

A. Wound Healing

Native substance P has been disclosed as useful in promoting wound healing. See, U.S. Pat. No. 5,616,562, U.S. Patent Application Number 2007015448, European Patent Application Number EP 1308165, Benrath et al., 1995, Neurosci. Lett. 200:17-20, Parenti, et al., 1996, Naunyn-Schmiedeberg's Arch. Pharmacol. 353: 475-481, Kahler, et al., 1993, Eur. J. Pharmacol. 249: 281-286, Ziche, et al., 1990, Pharmacol. Lett. 48: 7-11, Brain, 1997, Immunopharmacol. 37: 133-152 and Nilsson, et al., 1985, Nature 315: 61-63

Applicants have found that certain substance P analogs, as described herein, stimulate proliferation or differentiation of stem cells at lower concentrations (in some cases log scale less) that the native compound. While not being bound to any theory, applicants believe the substance P analogs described herein, including [Sar⁹Met(O₂)¹¹]-substance P, are more efficacious than substance P for several reasons. First, the substance P analogs have a greater affinity for the neurokinin-1 (NK1) receptor than substance P and thus more likely to initiate a secondary message signal. Sagan, et al. 1996, J. Pharmacol. Exper. Ther. 276: 1039-1048. In addition, as described elsewhere herein, the substance P analogs of the present invention are less prone to degradation than substance P, thus having a longer time period to exert biological effects. The preferred substance P analogs, including [Sar⁹Met(O₂)¹¹]-substance P, are specific to the NK1 receptor, whereas substance P can also activate the neurokinin-2 (NK2) and neurokinin-3 (NK3) receptors. Tousignant, et al. 1990, Brain Res. 524: 263-270.

Again, while not being bound to any theory, applicants believe the substance P analogs described herein, including [Sar⁹Met(O₂)¹¹]-substance P, exert biological effects both locally and systemically. When absorbed into the systemic circulation, the substance P analogs can contact hematopoietic stem cells (HSCs). The substance P analogs then promote differentiation of HSCs into granulocyte macrophage precursors, which can lead to increased circulating granulocyte and macrophage levels. See, Example 2.

In addition, the substance P analogs can also exert a local effect. At or near the wound site, the substance P analogs are believed to exert direct effects on mast cell degranulation, which increases growth factor production, including granulocyte macrophage-colony stimulating factor (GM-CSF) and interleukin-3 (IL-3). In addition, mast cell degranulation can also increase chemokine production, such as monocyte chemotactic protein-1 (MCP), CCL5, interleukin-8 (IL-8) which increases local granulocyte and macrophage populations. See also, Kulka et al., 2007, Immunol. 123: 398-410. When in the systemic circulation, the substance P analogs are believed to contact endothelial progenitor cells (EPCs) and promote differentiation of EPCs as well as promote mobilization of these cells to the wound site. By promoting EPC differentiation and mobilization, EPC populations in the wound site are increased. EPCs can increase and promote vasculogenesis and neovascularization further enhancing wound healing. Galiano, et al., 2004, Am. J. Pathol. 164: 1935-1947.

Macrophages play a critical role and can enhance the wound healing processes of debridement, antimicrobial action, matrix synthesis and regulation, cellular activation and neovascularization. Enhancing these processes can lead to an increase in the wound closure rate. Homspera® may also have a direct, activating effect on macrophages that enhances these healing processes as well.

The cells produced according to the methods described herein can be used for a variety of applications. For example, the methods provided herein can be used to stimulate in vivo proliferation or differentiation of stem cells that can be transferred to an ex vivo matrix or scaffold. A matrix or scaffold suitable for the methods would allow cell attachment, allow cell migration, deliver and retain cells and biochemical factors, enable diffusion of vital cell nutrients and expressed products and exert mechanical and biological influences to modify the behavior of the cell phase. The matrix or scaffold can be a synthetic extracellular matrix, natural extracellular matrix or synthetic. A natural extracellular matrix can be, for example, a tissue matrix comprised of collagen, fibronectin, cadaver tissue, basement membranes or placenta. See, for example, U.S. Pat. No. 7,311,904. Synthetic scaffolds can be, for example, polylactide (PLA), poly-D-, L-lactide-co-glycolide (PLGA), carbon fiber, calcium phosphate and the like. In one embodiment the scaffold can be a nanofiber scaffold (See, PuraMatrix™, 3DM Inc., Cambridge, Mass.). See also, U.S. Patent Publication Numbers 20070258948, 20060057720, 20060040386, 20060015961, 20060010509, 20060010508 and 20050158858.

In one embodiment, the cells can be used to stimulate wound healing or blood cell regeneration. In one embodiment, the methods can be used to stimulate tissue growth. Said tissue growth can be regeneration of, for example, human organs. In one embodiment, the cells can be used in treatment of a disorder or disease.

In one embodiment, cells and one or more substance P analogs can be added to a suspension, gel, matrix and/or another suitable carrier and used as a bandage. For example, a liquid bandage comprising progenitor cells and one or more substance P analogs in a liquid or gel that is able to polymerize can be placed over a wound to promote wound healing. In another embodiment, a bandage can be constructed that comprises progenitor cells and one or more substance P analogs in a matrix. The compositions and methods can affect any stage of wound healing, as described below. The compositions and methods of the present invention can be used to treat wounds present internally, for example, wounds on internal tissues or organs, or wounds present externally, for example, on an external surface or orifice.

In one embodiment, the bandage described herein can be used for the treatment of external full thickness or deep wounds. Such wounds are difficult to heal or close. In one embodiment the wound can be a traumatic wound, diabetic wound or decubitus ulcer.

Wound healing takes place in stages, including the inflammatory, proliferative and remodeling stages. In the inflammatory stage, neutrophils and macrophages clear bacteria and other debris from the wound site through phagocytosis and macrophages release factors that encourage the start of the proliferative phase. In the proliferative phase, angiogenesis, fibroplasia and granulation tissue formation, epithelialization and contraction occur. Angiogenesis is the formation of new blood vessels, which bring nutrients to the wound site. Fibroplasia is the migration of fibroblasts to the wound site. Fibroblasts deposit components of extracellular matrix (ECM) and collagen, important to the healing process. Granulation tissue consists of new blood vessels, fibroblasts, inflammatory cells, endothelial cells, myofibroblasts, and the components of a new, provisional ECM. Epithelialization occurs when epithelial cells (keratinocytes) migrate from surrounding tissue to the wound site and then proliferate to supply the cells needed to re-epithelialize the wound site. Contraction occurs when fibroblasts differentiate into myofibroblasts, which are similar to smooth muscle cells. Contraction of myofibroblasts causes the wound site to decrease in size. Finally, in the remodeling stage, the original disorganized strands of collagen are degraded and new, stronger, organized strands of collagen are formed in their place, increasing the tensile strength of the wound site. Unnecessary blood vessels are also removed through apoptosis. The foregoing description is not meant to provide a complete description of the process of wound healing. Many other steps which are known to those skilled in the art occur throughout the process of wound healing. The compositions and methods of the present invention may stimulate or improve any process involved in wound healing.

As stated above, fibroblasts play an important role in wound healing. Fibroblasts are the cells that form collagen, a primary component of the dermis that provides skin structure and support. Over time, fibroblasts become more inactive and collagen production slows. Substance P (RPKPQQFFGLM, SEQ ID NO:1) has been shown to augment cytokine-induced fibroblast proliferation. See, Cury et al., 2007J. Periodont. Res. 78(7): 1309-1315. Kahler, 1996, J. Cell Physiol. 166: 601-608, Katayama and Nishioka 1997, J. Derm. Sci. 15(3): 210-206, Kimball and Fisher 1998, Annals N.Y Acad. Sci. 540(1): 681-683 and Ziche et al., 1990, Br. J. Pharmacol. 100(1): 11-14. However, analogs of substance P may advantageously demonstrate resistance to degradation and thus have improved benefits in stimulating fibroblast proliferation, both directly and via facilitation of growth-factor driven proliferation.

Provided herein are methods for stimulating or promoting fibroblast proliferation or development, said methods comprising adding one or more substance P analogs to substantially purified fibroblast cells and stimulating growth, proliferation or development of said fibroblasts. The substantially purified fibroblast cells are preferably harvested from a donor. In a preferred embodiment, the methods are applied to autologous fibroblasts, that is, fibroblasts harvested from a donor are proliferated in culture with a media having one or more substance P analogs and then developed or substantially developed fibroblasts are administered to the original donor. In a preferred embodiment, the methods can be used for cosmetic purposes such as dermal fillers to nasolabial folds or to improve the appearance of scars. See, WO 2000/073418 and U.S. Pat. No. 5,866,167.

In one embodiment, the fibroblasts are mammalian. In one embodiment, the fibroblasts are porcine, equine or bovine. In a preferred embodiment, the fibroblasts are human. In one embodiment, the fibroblasts can be dermal fibroblasts.

Fibroblasts can be grown in culture as is known to those skilled in the art. See, Jarman-Smith, et al. 2004, Biochem. Engineering J. 20(2-3): 217-222, Wetzels, et al., 1998, Human Reproduction 13; 1325-1330; Smith et al., 1972, PNAS USA 69(11): 3260-3262, Watson et al., 1999, Arch. Facial Plast. Surg. 1: 165-170, WO 1998/36704. Fibroblast proliferation both in culture and in vivo is believed to occur through at least two mechanisms. One mechanism is via direct stimulation of fibroblasts in the dermis. Second, substance P analogs are also believed to be involved in growth-factor drive proliferation. In this regard, substance P analogs are believed to trigger or stimulate production or release of growth factors, mediators and the like that further promote fibroblast proliferation. Without being bound by any theory, it is believed that substance P analogs promote or stimulate angiogenesis thus increasing blood flow to wounds. Increased blood flow promotes infiltration of immune-boosting cells, such as macrophages, to promote wound healing. Furthermore, substance P analogs have demonstrated ability to stimulate proliferation or differentiation of stem cells, particularly granulocytes, to promote wound healing.

In one embodiment, fibroblasts can be contacted with one or more substance P analogs to stimulate fibroblast growth and proliferation. In another embodiment, the fibroblasts are a fibroblast culture and the culture media is a serum containing media. In one embodiment, proliferation occurs at about 12 hours, about 1 day, about 3 days or about 5 days in culture. In one embodiment, the amount of substance P analog to be added to the culture media can be from in an amount sufficient to achieve a final concentration of about 0.01 μM to about 10 μM. In certain embodiments, fibroblasts are cultured in the presence of about 5% FBS and the substance P analog for about 1, 2, 3, 4, 5, 6, 7, 8, 9, or 10 days. In certain embodiments, fibroblasts are cultured in the presence of about 0.5% FBS and the substance P analog for about 1, 2, 3, 4, 5, 6, 7, 8, 9, or 10 days. In certain embodiments, FBS is replaced with, for example, with a FBS substitute as commercially available from, for example, Valley Biomedical Products & Services, Inc.

Fibroblast stimulation in vitro can be accomplished by adding substance P analogs to the culture media of fibroblasts. Fibroblasts can be seeded in well plates in medium containing about 2.5%, about 5% about 7.5%, about 10% or about 15% fetal bovine serum (FBS).

In one embodiment, the methods provide ex vivo tissue remodeling. Three dimensional extracellular matrices have been developed as scaffolding for human cells. These matrices provide structural architecture to allow cellular growth in a three dimensional architecture. See, U.S. Pat. Nos. 7,311,904, 7,358,284, 7,338,517 and 7,108,721. In one embodiment, the methods provide for addition of substance P analogs to tissue matrices to promote fibroblast proliferation and differentiation. In a preferred embodiment, the tissue matrices are applied to a subject to promote external wound healing. External wounds (i.e. wounds to the dermis and epidermis of the skin) are described. Other types of wounds are within the scope of the present methods and compositions. For example, tissue matrices can be used to treat, for example, oral wounds wherein tissue remodeling of the gingival fibroblasts can be beneficial. In another example, the tissue matrix can be implanted within a subject to promote or replace damaged tissue.

In yet another embodiment, the methods provide for application of the substance P analogs to a wound. In a preferred embodiment, the substance P analogs are applied directly to traumatized or wounded tissue. In one preferred embodiment, the substance P analogs can be applied as a pharmaceutical composition such as a powder, gel, ointment, cream or spray, or via a device such as a dressing or bandage.

As will be apparent to one of skill in the art, the methods and compositions provided herein can be used with other methods and compositions known to promote fibroblast proliferation or wound healing. For example, the substance P analogs can be used in conjunction with, for example, epidermal growth factor or analogs thereof, (U.S. Pat. No. 7,084,246), wound cleansers such as cetylpyridinium chloride (U.S. Pat. No. 4,774,329), or bandages such as Algicell™ Ag antimicrobial alginate dressing (Derma Sciences, Princeton, N.J.). In a preferred embodiment, the substance P analogs are combined with a composition that allows application in a dressing having a high degree of conformance to the wound and surrounding tissue and would be able to be applied to hard to cover areas such as between fingers and toes or over joints. In one embodiment, the substance P analogs can be combined with a poly hydrogel composition.

In yet another aspect, the methods and compositions provide a kit comprising at least substance P analog and a second wound care item. In one embodiment, the kit comprises a substance P analog, wound debridement materials and compositions, and wound bandages. In one embodiment, the kit comprises one or more substance P analogs and a composition that promotes wound healing, such as epidermal growth factor or analogs thereof.

B. Diseases and Disorders

The methods described herein can be used to treat, prevent or ameliorate a stem cell disorder. In one embodiment the methods are used to treat, prevent or ameliorate an illness, disorder or disease such as bone marrow failure disorders, hemoglobinopathies, histiocytic disorders, inherited immune system disorders, inherited metabolic disorders, leukemias, lymphomas, myelodysplastic or myeloproliferative disorders, plasma cell disorders, inherited disorders, malignancies, phagocytic disorders and other disorders that can be treated or ameliorated with stem cells. Diseases or disorders that can be treated with the methods can be amegakaryocytosis, aplastic anemia, blackfan-diamond anemia, congenital cytopenia, congenital dyserythropoietic anemia, dyskeratosis congenital, Fanconi anemia, paroxysmal nocturnal hemoglobinuria (PNH), pure red cell aplasia, acute myelofibrosis, agnogenic myeloid metaplasia (myelofibrosis), polycythemia vera, essential thrombocythemia, beta thalassemia major, sickle cell disease, familial erythrophagocytic lymphohistiocytosis, hemophagocytosis, Langerhans' cell histiocytosis (hystiocytosis X), chronic granulomatous disease, congenital neutropenia, ataxia-telangiectasia, myelokathexis, bare lymphocyte syndrome, leukocyte adhesion deficiency, severe combined immunodeficiencies (SCID) (including adenosine deaminase deficiency, SCID with absence of T & B cells, SCID with absence of T cells, normal B cells), common variable immunodeficiency, bare lymphocyte syndrome, Chediak-Higashi syndrome, Kostmann syndrome, Omenn syndrome, purine nucleoside phosphorylase deficiency, reticular dysgenesis, Wiskott-Aldrich syndrome, X-linked lymphoproliferative disorder, adrenoleukodystrophy fucosidosis, Gaucher disease, Hunter's syndrome (MPS-H), Hurler's syndrome (MPS-IH), Krabbe disease, Lesch-Nyhan syndrome, mannosidosis, Maroteaux-Lamy syndrome (MPS-VI), metachromatic leukodystrophy, mucolipidosis II (I-cell disease), neuronal ceroid lipofuscinosis (Batten disease), Niemann-Pick disease, Sandhoff disease, San Filippo syndrome Morquio Syndrome (MPS-IV), Sly Syndrome, Beta-Glucuronidase deficiency (MPS-VII), andrenoleukodystrophy, Scheie syndrome (MPS-IS), sly syndrome, Tay Sachs, Wolman disease, Mucopolysaccharidoses (MPS), acute biphenotypic leukemia, acute lymphocytic leukemia (ALL), acute myelogenous leukemia (AML), acute undifferentiated leukemia, adult T cell leukemia, adult T cell lymphoma, chronic lymphocytic leukemia (CLL), chronic myelogenous leukemia (CML), Hodgkin's lymphoma, juvenile chronic myelogenous leukemia (JCML), juvenile myelomonocytic leukemia (JMML), myeloid/natural killer cell precursor acute leukemia, non-Hodgkin's lymphoma, polymphocytic leukemia, acute myelofibrosis, agnogenic myeloid metaplasia (myelofibrosis), amyloidosis, chronic myelomonocytic leukemia (CMML), essential thrombocythemia, polycythemia vera, refractory anemias (including refractory anemia with excess blasts (RAEB), refractory anemia with excess blasts in transformation (RAEB-T), refractory anemia with ringed sideroblasts (RARS), multiple myeloma, plasma cell leukemia, Waldenstrom's macroglobulinemia, cartilage-hair hypoplasia, Glanzmann thrombasthenia, amegakaryocytosis, congenital thrombocytopenia, congenital erythropoietic porphyria (Gunther disease) DiGeorge syndrome, osteopetrosis, brain tumors, Ewing sarcoma, neuroblastoma, ovarian cancer, breast cancer, neuroblastoma, renal cell carcinoma, rhabodomyosarcoma, small cell lung cancer, testicular cancer, thymoma (thymic carcinoma), chronic active Epstein barr, Evans syndrome, multiple sclerosis, rheumatoid arthritis, systemic lupus erythematosus, thymic dysplasia, Chediak-Higashi syndrome, chronic granulomatous disease, neutrophil actin deficiency, reticular dysgenesis, deafness, loss of hearing, diabetes, heart disease, liver disease, muscular dystrophy, Parkinson's disease, spinal cord injury or stroke.

In certain embodiments the stem cell disorder is not myeloproliferative disorders including, refractory anemias (including refractory anemia with excess blasts (RAEB), refractory anemia with excess blasts in transformation (RAEB-T) or refractory anemia with ringed sideroblasts (RARS). In certain embodiments the stem cell disorder is not drug induced or due to radiation.

C. Ex Vivo Expansion of Bone Marrow Cells

In one embodiment the methods provide for ex vivo expansion of substantial purified bone marrow mesenchymal stem cells. In one embodiment, the methods provide for maintenance of bone marrow cells in culture. The methods provide for long term in vitro cell culture of bone marrow cells by the addition of substance P analogs to the culture media. The substance P analogs, described herein, promote cell self-renewal without promoting differentiation. Thus the cells can be maintained in culture for extended periods of time or indefinitely without differentiation.

Since serum-free medium without growth factors is not capable of amplifying stem cells in vitro, the type of serum used, for example, fetal calf serum or human serum, allogeneic or autologous, serum or plasma can be an issue. The potential for contamination from prion, virus or zoonosis exists as does the potential for immunological reaction against xenogenic serum antigens. See, Berger et al., 2006, Stem Cells 24(12): 2888-90. The methods described herein are advantageous in that the risk of infection from zoonotic diseases is decreased.

Methods of bone marrow culture can be those known or developed including those described by Wolf et al., 1991, J. Immunol. 147(10): 3324-30, Gartner and Kaplan, 1980, Proc. Natl. Acad. Sci. 77(8): 4756-59, Berger et al., 2006, Stem Cells 24(12): 2888-90, Da Silva Meirelles and Nardi 2003, Brit. J. Haematol. 123(4) 702-1, Tamama et al., 2005, Stem Cells 24(3): 685-95, Martin et al., 1997, Endocrinology 138(10) 4456-62.

In one embodiment, the bone marrow cells are grown in a chemically denied media with a substance P analog. In one embodiment the substance P analog is present in an amount from about 10⁻³ to about 10⁻¹⁷ M. In a more preferred embodiment, the substance P analog is in an amount from about 10⁻⁶ to about 10⁻¹⁴ M.

Human liquid hematopoietic cultures which can be used in accordance with the invention can be grown at cell densities of from 10⁴ to 10⁹ cells/ml of culture, using standard known medium components such as, for example, IMDM, MEM, DMEM, RPMI 1640, Alpha Medium or McCoy's Medium, which can use combinations of serum albumin, cholesterol and/or lecithin, selenium and inorganic salts. As known in the art, these cultures can be supplemented with corticosteroids, such as hydrocortisone at a concentration of 10⁴ to 10⁻⁷ M, or other corticosteroids, such as cortisone, dexamethosone or solumedrol, at an equally potent dose. These cultures are typically maintained at a pH which is roughly physiologic, i.e. 6.9 to 7.4. The medium is typically exposed to an oxygen-containing atmosphere which contains from 4 to 20 vol. percent oxygen, preferably 6 to 8 vol. percent oxygen.

In one embodiment, the media can further have cytokines such as interleukin 1, interleukin 3, interleukin 6, interleukin 7, interleukin 8, interleukin 9, interleukin 10, interleukin 11, platelet derived growth factor (PDGF), epidermal growth factor (EGF), stem cell factor, granulocyte macrophage-colony stimulating factor (GM-CSF), granulocyte-colony stimulating factor (G-CSF), fibroblast growth factor-2, epidermal growth factor, transforming growth factor β, mast cell growth factor or erythropoietin.

In one embodiment, the methods provide for ex vivo expansion of granulocytes,

macrophages or platelets, said method comprising culturing bone marrow, umbilical cord blood or placental blood with a substance P analog. See, Qiu et al., 1999 J. Hematother. Stem Cell Res. 8(6): 609-18, Madkaikar et al., 2007, Acta Haematol. 118: 153-9. Proliferation or differentiation of blood cells could be useful for combating anemia, thrombocytopenia and leucopenia in humans.

In one embodiment, the methods provide for ex vivo expansion of bone marrow hematopoietic stem cells that have been mobilized from the bone marrow to the periphery using a mobilizing agent. In a preferred embodiment the mobilized cells are CD34+ cells. In one embodiment, the methods provide for harvesting of the CD34+ cells, expansion of the cells in media supplemented with a substance P analog and administration of the cells to the human.

In one embodiment the methods provide for promotion and maintenance of progenitor cells from human umbilical cord blood by the addition of a substance P analog to the culture media. See, Madkaikar et al., 2007, Acta Haematol. 118: 153-9, Broxmeyer et al., 1989 Proc. Natl. Acad. Sci. 86: 3828-32, incorporated herein by reference.

In one embodiment the methods provide for ex vivo expansion of HBMCs or cord blood cells wherein said expansion can be an increase in mean nucleated cell count, increase in mean CD34+ cell count, increase in cell colony count or an increase in viability. In one embodiment, there is an increase of about 20%, about 30%, about 40% or about 50% in mean nucleated cell count or CD34+ cell count. Greater increases are preferred. In one embodiment, the size of the HBMC colonies formed in clonal conditions in the presence of a substance P analog can be about 2 times, about 3 times or about 4 times larger than colonies formed in the absence of a substance P analog.

### Substance P and Analogs Thereof

As will be understood by those of skill in the art, substance P refers to peptide: Arg Pro Lys Pro Gln Gln Phe Phe Gly Leu Met (SEQ ID NO:1), or the single letter representation RPKPQQFFGLM (SEQ ID NO:1). As such, a substance P analog as used in the methods and compositions described herein refers to a substance P analog that comprises one or more amino acids substitutions relative to SEQ ID NO:1 and can either compete with substance P for binding to its receptor (NK-1) or agonize the NK-1 (neurokinin) receptor according to an assay conventional to the art, e.g., as described in Shue, et al., Bioorgan Med Chem Letters 2006, 16(4): 1065-1069. The amino acid substitutions can be conservative or non-conservative substitutions. Further, the amino acid substitutions can include substitutions of non-standard amino acids (e.g., amino acids other than the 20 amino acids normally encoded by the genetic code). In one example, the substance P analog can comprise norleucine (Nle). In yet another example, the substance P analog can comprise sarcosine (Sar) or N-methylglycine (MeGly). In yet another example, the substance P analog can comprise phenylalanine that is substituted with between 1 and 4 chlorines, more preferably 1 chlorine.

In one embodiment, the substance P analog is [Nle¹¹]-substance P, e.g., the substance P analog wherein the 11^{th} amino acid position is norleucine, i.e., the peptide: Arg Pro Lys Pro Gln Gln Phe Phe Gly Leu Nle (RPKPQQFFGLNle) (SEQ ID NO:2). In one embodiment, the substance P analog is [Pro⁹]-substance P, which refers to the substance P analog wherein the 9^{th} amino acid position is proline and has the sequence: Arg Pro Lys Pro Gln Gln Phe Phe Pro Leu Met (RPKPQQFFPLM) (SEQ ID NO:3). In one embodiment, the substance P analog is [Sar⁹]-substance P, which refers to the substance P analog wherein the 9^{th} amino acid position is Sarcosine or N-Methylglycine and has the sequence: Arg Pro Lys Pro Gln Gln Phe Phe MeGly Leu Met (RPKPQQFFMeGlyLM) (SEQ ID NO:4). In one embodiment, the substance P analog is [Tyr⁸]-substance P refers to the substance P analog wherein the 8^{th} amino acid position is tyrosine and has the sequence: Arg Pro Lys Pro Gln Gln Phe Tyr Gly Leu Met (RPKPQQFTGLM) (SEQ ID NO:5). [p-Cl-Phe^{7,8}]-substance P refers to the substance P analog wherein the Phenylalanine residue at positions 7 and 8 are chlorinated at the 4 position and has the sequence: Arg Pro Lys Pro Gln Gln Phe(4-Cl) Phe(4-Cl) Gly Leu Met-NH₂ (RPKPQQF(4-CL)F(4-CL)GLM) (SEQ ID NO:6). In one embodiment, the 11^{th} amino acid residue is Methionine sulfoxide, RPKPQQFFGLM(O) (SEQ ID NO:7). In one embodiment, the 9^{th} amino acid residue is Sarcosine and the 11^{th} residue is Methionine sulfoxide, RPKPQQFFMeGlyLM(O) (SEQ ID NO:8). In one embodiment, the 11^{th} amino acid residue is Methionine sulfone, RPKPQQFFGLM(O)₂ (SEQ ID NO:9). [Sar⁹, Met (O₂)¹¹]-substance P refers to the substance P analog wherein the 9^{th} amino acid position is Sarcosine or N-Methylglycine and the 11^{th} amino acid position is Met(O₂) and has the sequence:

(SEQ ID NO: 10)

Arg Pro Lys Pro Gln Gln Phe Phe MeGly Leu Met-O₂
(RPKPQQFFMeGlyLM-O₂).

In one embodiment, the methods provide for enhanced stem cell proliferation or differentiation in vitro or in vivo by the addition of a substance P analog wherein the substance P analog is of Formula (I):

(SEQ ID NO: 11)

Z₁-Xaa¹-Xaa²-Xaa³-Xaa⁴-Xaa⁵-Xaa⁶-Xaa⁷-Xaa⁸-Xaa⁹-
Xaa¹⁰-Xaa¹¹-Z₂ (I)

or a pharmaceutically acceptable salt thereof, wherein:
Xaa¹ is Arg, Lys, 6-N methyllysine or (6-N,6-N)dimethyllysine;

### Xaa² is Pro or Ala;

### Xaa³ is Lys, Arg, 6-N-methyllysine or (6-N,6-N)dimethyllysine;

### Xaa⁴ is Pro or Ala;

### Xaa⁵ is Gln or Asn;

### Xaa⁶ is Gln or Asn;

Xaa⁷ is Tyr, Phe or Phe substituted with chlorine at position 2, 3 or 4;
Xaa⁸ is Tyr, Phe, or Phe substituted with chlorine at position 2, 3 or 4;

### Xaa⁹ is Gly, Pro, Ala or N-methylglycine;

Xaa¹⁰ is Leu, Val, Ile, Norleucine, Met, Met sulfoxide, Met sulfone, N-methylleucine, or N-methylvaline;
Xaa¹¹ is Met, Met sulfoxide, Met sulfone, or Norleucine;

### Z₁ is R₂N— or RC(O)NR—;

Z₂ is —C(O)NR₂ or —C(O)OR or a salt thereof;
each R is independently —H, (C₁-C₆) alkyl, (C₁-C₆) alkenyl, (C₁-C₆) alkynyl, (C₅-C₂₀) aryl, (C₆-C₂₆) alkaryl, 5-20 membered heteroaryl or 6-26 membered alkheteroaryl; and
each “-” between residues Xaa¹ through Xaa¹¹ independently designates an amide linkage, a substitute amide linkage or an isostere of an amide.

In certain embodiments, the substance P analog comprises substance P (SEQ ID NO:1). In certain embodiments, the substance P analog consists of substance P (SEQ ID NO:1). In certain embodiments, the substance P analog is not substance P (SEQ ID NO:1).

In one embodiment, the substance P analog can be of Formula (I):

(SEQ ID NO: 11)

Z₁-Xaa¹-Xaa²-Xaa³-Xaa⁴-Xaa⁵-Xaa⁶-Xaa⁷-Xaa⁸-Xaa⁹-
Xaa¹⁰-Xaa¹¹-Z₂ (I)

wherein:

### Xaa¹ is Arg;

### Xaa² is Pro;

### Xaa³ is Lys;

### Xaa⁴ is Pro;

### Xaa⁵ is Gln;

### Xaa⁶ is Gln;

Xaa⁷ is Tyr, Phe or Phe substituted with chlorine at position 4;
Xaa⁸ is Tyr, Phe, or Phe substituted with chlorine at position 4;

### Xaa⁹ is Gly, Pro or N-methylglycine;

### Xaa¹⁰ is Leu; and

Xaa¹¹ is Met, Met sulfoxide, Met sulfone or Norleucine.

In a preferred embodiment, the substance P analog can be of Formula (I) as described herein wherein the “-” between residues Xaa¹ through Xaa¹¹ designates —C(O)NH—; Z₁ is H₂N—; and Z₂ is C(O)NH₂.

In certain embodiments, the substance P analog can be:

| | |
|---|---|
| RPKPQQFFGLM; | (SEQ ID NO: 1) |
| RPKPQQFFGLNle; | (SEQ ID NO: 2) |
| RPKPQQFFPLM; | (SEQ ID NO: 3) |
| RPKPQQFFMeGlyLM; | (SEQ ID NO: 4) |
| RPKPQQFTGLM; | (SEQ ID NO: 5) |
| RPKPQQF(4-Cl)F(4-Cl)GLM; | (SEQ ID NO: 6) |
| RPKPQQFFGLM(O); | (SEQ ID NO: 7) |
| RPKPQQFFMeGlyLM(O); | (SEQ ID NO: 8) |
| RPKPQQFFGLM(O₂) | (SEQ ID NO: 9) |
| or | |
| RPKPQQFFMeGlyLM(O₂). | (SEQ ID NO: 10) |

In an even more preferred embodiment, the substance P analog can be Z₁—RPKPQQFFMeGlyLM(O₂)—Z₂; wherein Z₁ is NH₂ and Z₂ is C(O)NH₂.

In one embodiment the methionine residue side chain sulfur (S) can be oxidated. In one embodiment the methionine is methionine sulfoxide (—NH—αCH(CO)—CH₂—CH₂—S(O)CH₃). In one embodiment the methionine is methionine sulfone or methionine S, S, dioxide, (—NH—αCH(CO)—CH₂—CH₂—S(O₂)CH₃), also referred to herein as Met(O)₂. It will be apparent to one skilled in the art that the amino (designated herein as Z₁) or carboxy terminus (designated herein as Z₂) of the substance P analogs can be modified. Included within the of the embodiments are “blocked” forms of the substance P analogs, i.e., forms of the substance P analogs in which the N- and/or C-terminus is blocked with a moiety capable of reacting with the N-terminal —NH₂ or C-terminal —C(O)OH. In some embodiments the N- and/or C-terminal charges of the substance P analogs can be an N-acylated peptide amide, ester, hydrazide, alcohol and substitutions thereof. In a preferred embodiment of the invention, either the N- and/or C-terminus (preferably both termini) of the substance P analogs are blocked. Typical N-terminal blocking groups include RC(O)—, where R is —H, (C₁-C₆) alkyl, (C₁-C₆) alkenyl, (C₁-C₆) alkynyl, (C₅-C₂₀) aryl, (C₆-C₂₆) alkaryl, 5-20 membered heteroaryl or 6-26 membered alkheteroaryl. Preferred N-terminal blocking groups include acetyl, formyl and dansyl. Typical C-terminal blocking groups include —C(O)NRR and —C(O)OR, where each R is independently defined as above. Preferred C-terminal blocking groups include those wherein each R is independently methyl. In another preferred embodiment the C-terminal group is amidated.

Substituted amides generally include, but are not limited to, groups of the formula —C(O)NR—, wherein R is (C₁-C₆) alkyl, substituted (C₁-C₆) alkyl, (C₁-C₆) alkenyl, substituted (C₁-C₆) alkenyl, (C₁-C₆) alkynyl, substituted (C₁-C₆) alkynyl, (C₅-C₂₀) aryl, substituted (C₅-C₂₀) aryl, (C₆-C₂₆) alkaryl, substituted (C₆-C₂₆) alkaryl, 5-20 membered heteroaryl, substituted 5-20 membered heteroaryl, 6-26 membered alkheteroaryl and substituted 6-26 membered alkheteroaryl.

Amide isosteres generally include, but are not limited to, —CH₂NH—, —CH₂S—, —CH₂CH₂—, —CH═CH— (cis and trans), —C(O)CH₂—, —CH(OH)CH₂— and —CH₂SO—. Compounds having such non-amide linkages and methods for preparing such compounds are well-known in the art (see, e.g., Spatola, March 1983, Vega Data Vol. 1, Issue 3; Spatola, 1983, “Peptide Backbone Modifications” In: Chemistry and Biochemistry of Amino Acids Peptides and Proteins, Weinstein, ed., Marcel Dekker, New York, p. 267 (general review); Morley, 1980, Trends Pharm. Sci. 1:463-468; Hudson et al., 1979, Int. J. Prot. Res. 14:177-185 (—CH₂NH—, —CH₂CH₂—); Spatola et al., 1986, Life Sci. 38:1243-1249 (—CH₂S—); Hann, 1982, J. Chem. Soc. Perkin Trans. 1. 1:307-314 (—CH═CH—, cis and trans); Almquist et al., 1980, J. Med. Chem. 23:1392-1398 (—COCH₂—); Jennings-White et al., Tetrahedron. Lett. 23:2533 (—COCH₂—); European Patent Application EP 45665 (1982) CA 97:39405 (—CH(OH)CH₂—); Holladay et al., 1983, Tetrahedron Lett. 24:4401-4404 (—C(OH)CH₂—); and Hruby, 1982, Life Sci. 31:189-199 (—CH₂—S—).

Additionally, one or more amide linkages can be replaced with peptidomimetic or amide mimetic moieties which do not significantly interfere with the structure or activity of the peptides. Suitable amide mimetic moieties are described, for example, in Olson et al., 1993, J. Med. Chem. 36:3039-3049.

### Compositions

In one embodiment, the embodiments described herein provide compositions for administration of a substance P analog to prevent, treat or ameliorate injuries, diseases or disorders associated with decreased stem cell activity. In one embodiment, the composition comprises an effective amount of a substance P analog according to Formula (I) as described herein.

Pharmaceutical compositions of the substance P analogs comprise a therapeutically effective amount of a compound described herein, formulated together with one or more pharmaceutically acceptable carriers. As used herein, the term “pharmaceutically acceptable carrier” or “carrier” refers to a non-toxic, inert solid, semi-solid or liquid filler, diluent, encapsulating material or formulation auxiliary of any type. Some examples of materials that can serve as pharmaceutically acceptable carriers are sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil; safflower oil; sesame oil; olive oil; corn oil and soybean oil; glycols; such a propylene glycol; esters such as ethyl oleate and ethyl laurate; agar, buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator. The pharmaceutical compositions can be administered to humans and other animals orally, rectally, parenterally, intracisternally, intravaginally, intraperitoneally, topically (as by powders, ointments, or drops), bucally, or as an oral or nasal spray, or a liquid aerosol or dry powder formulation for inhalation.

Injectable parenteral preparations, for example, sterile injectable aqueous or oleaginous suspensions can be formulated according to the known art using suitable dispersing or wetting agents and suspending agents. The sterile injectable preparation can also be a sterile injectable solution, suspension or emulsion in a nontoxic parenterally acceptable diluent or solvent, for example, as a solution in 1,3-butanediol. Among the acceptable vehicles and solvents that can be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid can be used in the preparation of injectables.

The injectable formulations can be sterilized, for example, by filtration through a bacterial-retaining filter, or by incorporating sterilizing agents in the form of sterile solid compositions that can be dissolved or dispersed in sterile water or other sterile injectable medium prior to use.

In order to prolong the effect of a drug, it is often desirable to slow the absorption of the drug from subcutaneous or intramuscular injection. This can be accomplished by the use of a liquid suspension of crystalline or amorphous material with poor water solubility. The rate of absorption of the drug then depends upon its rate of dissolution that, in turn, can depend upon crystal size and crystalline form. Alternatively, delayed absorption of a parenterally administered drug form can be accomplished by dissolving or suspending the drug in an oil vehicle. Injectable depot forms are made by forming microencapsule matrices of the drug in biodegradable polymers such as polylactide-polyglycolide. Depending upon the ratio of drug to polymer and the nature of the particular polymer employed, the rate of drug release can be controlled. Examples of other biodegradable polymers include poly(orthoesters) and poly(anhydrides).

Depot injectable formulations can also be prepared by entrapping the drug in liposomes or microemulsions that are compatible with body tissues.

In preferred embodiments, the parenteral composition can be administered intravenously, intramuscularly, subcutaneously or intradermally.

Intravenous, aerosol inhalation, topical, intratracheal, intrabronchial, intranasal, subcutaneous, sublingual, and oral administrations can be used. Suitable concentration ranges of substance P or its bioactive analog in an aerosol administered is between about 0.1 μM and about 5000 mM, Exemplary concentrations which can be used include about 1 mM, about 10 mM, about 50 mM, about 75 mM, about 100 mM, about 300 mM and about 1000 mM. For intramuscular injections, a volume of about 0.1 to 1.0 ml/kg of body weight can be used.

One skilled in the art can routinely determine dosages of substance P analogs for use in the methods and compositions described herein according to conventional parameters such as, for example, mass, distribution and clearance rates, etc. Doses will generally be from about 0.5 ng/kg to about 500 mg/kg.

Solid dosage forms for oral administration include capsules, tablets, pills, powders, and granules. In such solid dosage forms, the active compound can be mixed with at least one inert, pharmaceutically acceptable excipient or carrier such as sodium citrate or dicalcium-phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, acetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form can also comprise buffering agents.

Solid compositions of a similar type can also be employed as fillers in soft and hard-filled gelatin capsules using such excipients as lactose or milk sugar as well as high molecular weight polyethylene glycols and the like.

The solid dosage forms of tablets, dragees, capsules, pills, and granules can be prepared with coatings and shells such as enteric coatings and other coatings well known in the pharmaceutical formulating art. They can optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Solid compositions of a similar type can also be employed as fillers in soft and hard-filled gelatin capsules using such excipients as lactose or milk sugar as well as high molecular weight polyethylene glycols and the like.

The active compounds can also be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound can be admixed with at least one inert diluent such as sucrose, lactose or starch. Such dosage forms can also comprise, as is normal practice, additional substances other than inert diluents, e.g., tableting lubricants and other tableting aids such a magnesium stearate and microcrystalline cellulose. In the case of capsules, tablets and pills, the dosage forms can also comprise buffering agents. They can optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Absorption through the gastrointestinal tract can be accomplished with peptides particularly if formulated in an appropriate composition. For example, peptides, such as the substance P analogs can be made in a pro-drug composition to provide oral absorption. See, Borchardt 1999, J. Controlled Release 62(1-2): 231-238, Catnach et al., 1994, Gut 35(4): 441-444. In another embodiment, the oral routes of administration (including but not limited to ingestion, buccal and sublingual routes) can be used. In preferred embodiments, appropriate formulations (e.g., enteric coatings) are used to avoid or minimize degradation of the active ingredient, e.g., in the harsh environments of the oral mucosa, stomach and/or small intestine.

Liquid dosage forms for oral administration include pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Compositions for rectal or vaginal administration are preferably suppositories that can be prepared by mixing the compounds of this invention with suitable non-irritating excipients or carriers such as cocoa butter, polyethylene glycol or a suppository wax which are solid at ambient temperature but liquid at body temperature and therefore melt in the rectum or vaginal cavity and release the active compound.

Dosage forms for topical or transdermal administration of a compound include ointments, pastes, creams, lotions, gels, powders, solutions, sprays, inhalants or patches. The active component can be admixed under sterile conditions with a pharmaceutically acceptable carrier and any needed preservatives or buffers as may be required. Ophthalmic formulations, ear drops, and the like are also contemplated as being within the scope of these embodiments.

The ointments, pastes, creams and gels can contain, in addition to an active compound of this invention, excipients such as animal and vegetable fats, oils, waxes, paraffins, starch, tragacanth, cellulose derivatives, polyethylene glycols, silicones, bentonites, silicic acid, talc and zinc oxide, or mixtures thereof.

Compounds and compositions can also be formulated for use as topical powders and sprays that can contain excipients such as lactose, talc, silicic acid, aluminum hydroxide, calcium silicates and polyamide powder, or mixtures of these substances. Sprays can additionally contain customary propellants such as chlorofluorohydrocarbons.

Transdermal patches have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

Pharmaceutical compositions can also be formulated for delivery as a liquid aerosol or inhalable dry powder. Liquid aerosol formulations can be nebulized predominantly into particle sizes that can be delivered to the terminal and respiratory bronchioles where bacteria reside in patients with bronchial infections, such as chronic bronchitis and pneumonia. Pathogenic bacteria are commonly present throughout airways down to bronchi, bronchioli and lung parenchema, particularly in terminal and respiratory bronchioles. During exacerbation of infection, bacteria can also be present in alveoli. Liquid aerosol and inhalable dry powder formulations are preferably delivered throughout the endobronchial tree to the terminal bronchioles and eventually to the parenchymal tissue.

Aerosolized formulations can be delivered using an aerosol forming device, such as a jet, vibrating porous plate or ultrasonic nebulizer, preferably selected to allow the formation of aerosol particles having with a mass medium average diameter predominantly between 1 to 5 μm. Further, the formulation preferably has balanced osmolarity ionic strength and chloride concentration, and the smallest aerosolizable volume able to deliver effective dose of the compounds to the site of the delivery. Additionally, the aerosolized formulation preferably does not impair negatively the functionality of the airways and does not cause undesirable side effects.

Aerosolization devices suitable for administration of aerosol formulations, for example, jet, vibrating porous plate, ultrasonic nebulizers and energized dry powder inhalers, that are able to nebulize the formulation into aerosol particle size predominantly in the size range from 1-5 μm. Predominantly means that at least 70% but preferably more than 90% of all generated aerosol particles are 1 to 5 μm range. A jet nebulizer works by air pressure to break a liquid solution into aerosol droplets. Vibrating porous plate nebulizers work by using a sonic vacuum produced by a rapidly vibrating porous plate to extrude a solvent droplet through a porous plate. An ultrasonic nebulizer works by a piezoelectric crystal that shears a liquid into small aerosol droplets. A variety of suitable devices are available, including, for example, AeroNeb and AeroDose vibrating porous plate nebulizers (AeroGen, Inc., Sunnyvale, Calif.), Sidestream7 nebulizers (Medic-Aid Ltd., West Sussex, England), Pari LC7 and Pari LC Star7 jet nebulizers (Pari Respiratory Equipment, Inc., Richmond, Va.), and Aerosonic (DeVilbiss Medizinische Produkte (Deutschland) GmbH, Heiden, Germany) and UltraAire7 (Omron Healthcare, Inc., Vernon Hills, Ill.) ultrasonic nebulizers.

The substance P analogs can be advantageously administered as a liquid dosage form at a concentration between about 0.1 μM and 1M. More preferably from about 0.1 mM to about 100 mM. In an even more preferred embodiment, the substance P analogs can be administered based on the subject's weight. In one embodiment, the substance P analog is administered at a dose of about 0.01 mg/kg to about 10 mg/kg. In a more preferred embodiment, the compositions are administered at a dose of about 0.05 mg/kg to about 5 mg/kg. Other exemplary dosage forms include about 1 mL of about 100 mM substance P analog solution, about 1 mL of about 1 mM substance P analog solution or about 1 mL of about 10 μM substance P analog solution administered parenterally or by inhalation.

Methods of formulation are well known in the art and are disclosed, for example, in Remington: The Science and Practice of Pharmacy, Mack Publishing Company, Easton, Pa., 19th Edition (1995). Pharmaceutical compositions for use in the present invention can be in the form of sterile, non-pyrogenic liquid solutions or suspensions, coated capsules, suppositories, lyophilized powders, transdermal patches or other forms known in the art. It will be appreciated that the preferred route of administration and thus the type of pharmaceutical composition can vary with the condition, age and compliance of the recipient.

### Administration

The methods and compositions can be administered in a frequency and duration for prevention or amelioration of injuries, diseases and disorders associated with decreased stem cell activity. In one embodiment, the compositions can be administered one time (e.g. single dose). In one embodiment, the compositions can be administered multiple times, for example concomitantly with a medicament or following a medication regimen, for example. In one embodiment, the composition can be given hours, days, weeks or even months after a medicinal or therapeutic regimen (i.e. chemotherapy or radiation therapy). In one embodiment, the compositions can be administered intermittently, for example, every 3 days, every 7 days, every 14 days, every 30 days, every 60 days, every 90 days, every 180 days, every 360 days and the like.

The substance P analogs can be used in combination with one or more biological response modifiers. In a preferred embodiment, the biological response modifier can increase hematopoiesis. In one embodiment, the biological response modifier and the substance P analogs can be administered contemporaneously, for example, on the same day. In one embodiment, the biological response modifier and the substance P analog can be administered at intervals, based on the radiation schedule of a human. For example, the biological response modifier can be given on Day 1 and the substance P analog, with or without a concomitant biological response modifier, can be administered on Day −3, Day −1, Day 2, Day 3, Day 7, Day 10 or Day 14 of treatment. Such combinations can be administered either before or after radiation treatments. Such combinations can also be administered either before or after the myelosuppression is manifested.

According to the methods of treatment of the present invention, an injury, disease or disorder is treated or prevented in a patient such as a human or lower mammal by administering to the patient a therapeutically effective amount of a compound of the invention, in such amounts and for such time as is necessary to achieve the desired result. By a “therapeutically effective amount” of a compound of the invention is meant a sufficient amount of the compound to treat an injury, disease or disorder, at a reasonable benefit/risk ratio applicable to any medical treatment. It will be understood, however, that the total daily usage of the compounds and compositions of the present invention will be decided by the attending physician within the scope of sound medical judgment. The specific therapeutically effective dose level for any particular patient will depend upon a variety of factors including the disorder being treated and the severity of the disorder, the activity of the specific compound employed; the specific composition employed; the age, body weight, general health, sex and diet of the patient; the time of administration, route of administration, and rate of excretion of the specific compound employed; the duration of the treatment, drugs used in combination or coincidental with the specific compound employed; and like factors well known in the medical arts.

### Formulations for Embodiments Involving Treatment of Wounds

The compositions comprise substance P analogs according to Formula I as described herein in a physiologically-acceptable carrier. In certain embodiments the composition is suitable for topical application. In certain embodiments, the composition is suitable for implantation or partial implantation in a subject. The compositions can contain from about 0.01 fM to about 10 mM of substance P analog, usually from about 0.01 pM to about 1 mM substance P analog and more usually containing from about 0.1 pM to 100 mM substance P analog.

The carrier can vary depending on the intended area to be treated and type of wound. For application to the skin, a cream or ointment is usually preferred. Suitable bases known in the art that can be used include lanolin, Silvadene™ (Marion) and Aquaphor™ (Duke).

In one embodiment the carrier can be a controlled or sustained release carrier that permits the slow or controlled release of the substance P analogs to one or more sites where a therapeutic or ameliorative effect can occur. In one embodiment the controlled release carrier can be a polymeric carrier, such as, for example, hyaluronic acid, chondroitin, hydroxymethyl cellulose, paraffin, cetyl alcohol, polyethylene glycol, gelatin, sodium alginate, methyl cellulose, carboxymethyl cellulose, plastibase hydrophilic gelatin, dextrin, steryl alcohol, polyethylene glycol, polyvinyl alcohol, methoxyethylene-maleic anyhydride, nanoparticles, liposomes, or combinations thereof.

In one embodiment the composition can further comprise a medicinal compound that can exert a therapeutic or ameliorative effect on a wound. In preferred embodiments the medicinal compound can be an antibacterial, antifungal, silver or cetypyridinium (U.S. Pat. No. 4,774,329).

In a preferred embodiment the carrier is a polygalacturanic acid. In a more preferred embodiment, the carrier is an alpha 1-4 linked polygalacturanic acid. In another preferred embodiment the substance P analogs can be incorporated into an aloe pectin carrier such as that described in U.S. Pat. Nos. 6,274,548, 6,313,103, 5,929,051 and 7,022,683.

In certain embodiments the substance P analogs can be incorporated into natural and synthetic bandages and other wound dressings to provide for continuous exposure of a wound to the substance P analog.

In one embodiment the composition can be a liquid bandage comprising a substance P analog. Such an embodiment can be a liquid or gel that is able to substantially increase in viscosity when applied to a wound.

In one embodiment the substance P analog can be impregnated into a three-dimensional (3D) matrix or bandage. Such matrices and bandages are known in the art and some are available commercially. For example Ng describes a perfusable 3D cell-matrix tissue culture chamber for use with nanoparticles. Ng, C. P. and Pun, S. H. 2007, Biotech. Bioengineer. 99(6): 1490-1501. Commercially available 3D matrices include reconstituted basement membrane extract such as Matrigel™ (Becton Dickson) and Cultrex™ (Trevigen), interstitial matrix components such as collagen, fibrinogen and fibrin available from Millipore, Becton Dickson, Sigma, semi-synthetic hydrogels such as PuraMatrix Peptide Hydrogel™ (Becton Dickson) and Extracel™ and HyLink™ (Glycosan), and AlgiMatrix 3D Culture System (Invitrogen).

In certain embodiments the compositions can comprise substance P analogs with stem cell tissue constructs. See, Grayson et al., 2004, Biotechnol. Prog. 20(3): 905-912, Heike 2007, Curr. Pharm. Design, 13(35): 3597-3607, Laporte and Shea, 2007, Adv. Drug Deliv. Rev. 59(4-5): 292-307, Lutolf, et al., 2003, Proc. Natl. Acad. Sci. 2003 100(9): 5413-5418. In a preferred embodiment, the tissue matrices can be comprised of placental stem cells or human amniotic membrane allograft. See, U.S. Pat. No. 7,311,904 and Acelagraft™ (Celgene).

In a preferred embodiment, the compounds are a bandage or 3D matrix impregnated with one or more substance P analogs and one or more cells capable of growth or replication. In one embodiment, the cells are naïve cells, stem cells or progenitor cells. In another embodiment, the stem cells are placental, umbilical cord blood, embryonic or adult stem cells. In a more preferred embodiment, the stem cells are cutaneous epithelial stem cells or endothelial progenitor cells.

In certain embodiments, the 3D bandage or matrix described herein can be used for the treatment of full thickness or deep wounds. Stimulation of differentiation or proliferation of fibroblast, endothelial or endovascular cells within the matrix can allow for migration of said cells to the wound area. In such an embodiment, collagen formation and revascularization can enhance, improve or promote wound healing of deep wounds. In one embodiment the wound can be a traumatic wound, diabetic wound or decubitus ulcer.

Concentrations of the substance P analogs in 3D bandages or matrices can be about 0-1 pg/ml to about 100 pg/ml. In a more preferred embodiment the concentration can be about 1 pg/ml to about 10 ng/ml. In an even more preferred embodiment the concentration can be about 5 pg/ml to about 100 pg/ml.

In certain embodiments the compositions can further comprise a variety of growth promoting compounds such as cytokines, lymphokines, interleukins, chemokines and growth factors. In certain embodiments the growth factors can be, for example, epidermal growth factor, platelet-derived growth factor (including platelet-derived growth factor-B), insulin, insulin-like growth factor, transforming growth factor-β, nerve cell growth factor, fibroblast growth factor, vascular endothelial growth factor, granulocyte-macrophage colony stimulating factor, neurotropins, erythropoietin, thrombopoietin, myostatin, keratinocyte growth factor, bone morphogenic proteins, activins and the like. The concentrations of other growth factors can generally be about 0.01 pM to about 1 mM.

### Kits

In one embodiment the invention provides a kit for administering a substance P analog. Such a kit can have, for example, a device for administration (e.g. a syringe) and at least one substance P analog.

In one embodiment provided herein are a kit for administering a substance P analog and a drug. For example, such a kit can comprise both an anti-cancer drug and at least one substance P analog and optionally, one or more biological response modifier. In certain embodiments, the substance P analog can be of Formula I as described herein. The drug and substance P analog can be in separate, or divided or undivided containers. The two agents can be in liquid, dried, lyophilized, or frozen form, as is convenient for the end user and good for shelf life. The treatments can be administered at one time or sequentially, over a period of, for example, one day, one week, one month, six months or twelve months.

### EXAMPLES

The following examples are intended to illustrate the present invention and are not intended to limit the invention in any way.

### Example 1

### Half-Life of Plasma Homspera® Relative to Native Substance P

The objective was to determine the half-life of Homspera® (RPKPQQFFMeGlyLM(O₂)—NH₂ (SEQ. ID. NO:10)) in plasma from three animal species.

Frozen plasma from mice, human and non-human primates (designated hereinafter as primates for simplicity) was obtained from Biochemed (Winchester, Va.) (human: Lot BC061107-07, primate: Lot CYNBREC-27070, mouse: Lot S-74242). EDTA was added as an anticoagulant during isolation of the plasma for all samples.

The plasma was thawed and 990 μL was added to a 1.5 mL microcentrifuge vial. To have a final concentration of Homspera® in the plasma, two different stock solutions at either 1 mg/mL or 10 mg/mL were prepared using phosphate buffered saline (PBS), pH 7.4 at a 1× concentration. Ten μL of a 1 mg/mL solution were added to 990 μL of plasma for a final Homspera® concentration of 7 μM and samples were vortexed to mix. Ten μL of a 10 mg/mL solution were added to 990 μL of plasma for a final Homspera® concentration of 70 μM and samples were vortexed to mix. In a 96-deep-well plate, 50 μL of either the 7 μM or the 70 μM plasma samples were mixed with 50 μL of a 1×PBS, pH 7.4 solution.

For the preliminary half-life assessment, the 96-well plate was placed in a 37° C. oven for 0, 3, 10, 60, 120, and 180 min. Each sample per time point was terminated with the addition of 400 μL of 450 ng/mL glyburide in 90% acetonitrile (ACN) and 0.1% formic acid in water. Each sample was tested in duplicate. The plate was centrifuged at 4,000 rpm for 10 minutes and the supernatant was transferred to another 96-well plate. The supernatants were stored at −80° C. while other time points were being collected. Samples were evaporated using a Turbovap®and then reconstituted with 50 μL of 10 μg/mL of Pro⁹-substance P (SEQ. ID. NO:3) in 40% acetonitrile and 0.1% formic acid in water. Pro⁹-substance P functioned as an internal standard. Once the preliminary assessment was completed (data not shown), the time points were refined for the definitive portion. The time points assessed for the definitive study were 0, 10, 20, 30, 60, and 120 minutes. Samples were analyzed and quantitated by liquid chromatography/mass spectrometry (LC/MS).

The MS instrument was manufactured by Applied Biosystems (model: API 4000) and the LC portion of the instrument included a high pressure liquid chromatography (HPLC) pump made by Shimadzu (part number: LC-10ADvp) and an Agilent Poroshell HPLC column (part number 300 SB-C18 (2.1×75 mm). The system solvent consisted of two different mobile phases, designated as A or B, to resolve Homspera®. Mobile phase A was 95% H₂O, 5% ACN and 0.1% formic acid, and mobile phase B was 90% ACN, 10% H₂O, and 0.1% formic acid. The program used for separation on the HPLC column was a linear gradient from 0 to 1.5 minutes going from 0% B to 50% B, and from 50% B to 90% B at 1.5 minutes to 1.6 minutes, a hold at 90% B from 1.6 minutes to 2 minutes followed by a gradient from 90% B to 0% B until 2.1 minutes and a hold at 0% B from 2.1 to 2.5 minutes. The LC/MS was programmed to operate using a flow rate of 0.6 mL/min and 5 μL of sample were injected onto the HPLC column for each run. Additionally, the instrument was equipped with a turbo ion spray source, which was set to operate in the positive ion interface and multiple reaction monitoring acquisition modes. The source temperature was 500° C. and the total run time was 2.5 minutes. The parent/daughter ion pair of Homspera® was 698.8/348.4 and was 695.2/211.3 for the internal standard, Pro⁹-substance P.

At the lower concentration, 7 μM, the mean half-life in mice was 33 minutes (st. dev. 3). The mean half-life for humans and primates at 7 μM was very similar with a mean half-life in primates of 37 minutes (st. dev. 4). At higher concentrations, 70 μM, the mean mouse half-life was 38 minutes. (st. dev. 1). The mean half-life for primates at 70 μM was 59 minutes (st. dev. 6) and 66 minutes for humans (st. dev. 0). Without being bound to any theory, it is proposed that the higher concentration of Homspera® saturated the ex vivo system perhaps by binding to other proteins in the plasma that would stabilize or protect Homspera® from enzymatic degradation. Accordingly, it is concluded the half-life of Homspera® is between 30-60 minutes for the three species examined.

TABLE 1
Ex Vivo Homspera ® Half-Life in
Plasma in Three Animal Species.

| | |
|---|---|
| 7 μM | 70 μM |

| Species | Mean | St. dev. | Mean | St. dev |
|---|---|---|---|---|
| Mouse | 33 | 3 | 38 | 1 |
| Primate | 37 | 4 | 59 | 6 |
| Human | 35 | 4 | 66 | 0 |

With regard to substance P, Berger et al., reported that native substance P is rapidly degraded in rat brain fractions and in human plasma ex vivo. Berger et al., 1979, Biochem. Pharmacol. 28: 3173-3180. At concentrations below 10⁻⁷M, the native peptide had a half-life of 9.3 minutes when incubated in a 1 mg/mL rat brain homogenate fraction. The half-life of the native peptide in plasma, ex vivo, was 24 minutes.

Blumberg and Teichberg determined native substance P has a half-life of about 5 minutes (±2 min) when 0.2 μM was incubated in 1 mg/mL rat brain homogenate. Blumberg and Teichberg, 1979, Biochem. Biophys. Res. Comm. 90(1): 347-354.

### Example 2

### The Exemplary Substance P Analog Homspera® Stimulates Cellular Proliferation and Differentiation Following Radiation Treatment

This study was done to determine the effect of treating irradiated mice with an exemplary substance P analog.

A. Materials and Methods

Homspera® was provided by ImmuneRegen via CSBio, Inc. (Menlo Park, Calif., catalog number CS2663) as a lyophilized powder of the trifluoroacetate salt. The sample was stored at −20° C. until solubilized. Homspera® was dissolved in dilute sterile saline and dilute acetic acid to obtain a solution of 300 μM concentration.

Seventy-two (72) Balb/c mice of age 5-6 weeks and normal physiological state (Taconic) were separated into 4 groups: Non-irradiated control (or Non-treatment control) (n=12), Irradiated control (vehicle controls) (n=20), Irradiated/Treated pre-exposure (n=20), and Irradiated/Treated post-exposure (n=20). Animals were housed individually in ventilated microisolator cages (4-5 mice per cage), fed ad libitum Lab Diet pellets, and acclimated for 5-7 days prior to treatment. On Day 1, animals were placed into the X-ray irradiator (RadSource 2000) for 4 minutes. Non-irradiated controls received no radiation exposure while the irradiated controls were exposed to radiation at the level of 1 Gy/minute. Animals were either treated with vehicle control or 300 μM Homspera® in the same vehicle solution. The Non-irradiated control group and Irradiated control group were administered 25 μL of sterile saline intranasally daily for 7 days following radiation exposure. Animals treated with Homspera® pre-radiation exposure were administered 25 μL of 300 μM solution intranasally 1 day prior to radiation exposure and daily thereafter for 7 days. Animals treated with Homspera® post-radiation exposure were administered 25 μL of 300 μM solution intranasally daily for 7 days following radiation exposure as described in Table 2.

TABLE 2
Study Design

| | | | Homspera ® | Homspera ® |
|---|---|---|---|---|
| | | Vehicle | Pre-treatment | Post-treatment |
| Group | N | Control | (Day 0) | (Daily for 7 days) |
| 1. Non-irradiation | 12 | X | | |
| control | | | | |
| 2. Irradiated | 20 | X | | |
| control | | | | |
| 3. Homspera ® | 20 | | X | X |
| 4. Homspera ® | 20 | | | X |

Following radiation exposure, gross observations were made at least once daily. Animal body weights were recorded at Days 1, 2, 3, 4, 5, 9, and 12 for all irradiated animals. Three mice from each group, including controls, were sacrificed at each timepoint listed in Table 2 or when each mouse became moribund. The remaining mice, 8 each from the Irradiated control, Irradiated/Treated pre-exposure, and Irradiated/Treated post-exposure groups were observed for survival until Day 30 or when moribund.

TABLE 3
Blood collection timepoints

| Group | N* | Timepoints |
|---|---|---|
| Non-irradiated control | 12 | a) 3-6 hours post irradiation of |
| (vehicle control) | | treatment group |
| | | b) 12-18 hours post 1^{st} collection |
| | | c) 24 hours post 2^{nd} collection |
| | | d) 48 hours post 3^{rd} collection |
| Irradiated control | 12 | e) 3-6 hours post irradiation |
| | | f) 12-18 hours post 1^{st} collection |
| | | g) 24 hours post 2^{nd} collection |
| | | h) 48 hours post 3^{rd} collection |
| Homspera ® at Day 0 and | 12 | i) 3-6 hours post irradiation |
| daily for 7 days | | j) 12-18 hours post 1^{st} collection |
| | | k) 24 hours post 2^{nd} collection |
| | | l) 48 hours post 3^{rd} collection |
| Homspera ® daily for 7 | 12 | m) 3-6 hours post irradiation |
| days | | n) 12-18 hours post 1^{st} collection |
| | | o) 24 hours post 2^{nd} collection |
| | | p) 48 hours post 3^{rd} collection |

*Three mice sacrificed per timepoint

Deaths and unanticipated adverse reactions were reported to the institutional veterinarian as soon as noted. The mice were sacrificed by regulated CO₂ upon the animal being moribund. Mice were considered moribund if one or more of the following criteria were met: 1) loss of body weight of 20% or greater in a 1 week period; 2) prolonged, excessive diarrhea leading to excessive weight loss (>20%); 3) persistent wheezing and respiratory distress; 4) extreme lethargy; 5) dehydration indicated by loose skin; 6) fever indicated by shivering or 7) prolonged or excessive pain or distress observed as prostration, hunched posture, paralysis, paresis, distended abdomen, ulcerations, abscesses, seizures or hemorrhages.

The percentage of animal mortality and time to death were recorded for every group in the study.

B. Results

1. Animal Body Weights:

Animal body weights for non-irradiated controls were not recorded. Animal weights for all irradiated groups trended to decrease similarly to roughly 90% total body weight by Day 5 following radiation exposure. Animals exposed to radiation and treated with Homspera® (post-irradiation treatment) were observed to have a slight recovery in lost body weight by Day 9. However, pre-irradiation treatment animals and irradiated control (vehicle control) animals continued to lose weight until moribund or sacrificed at Day 12. Irradiated control animals lost 16.1% (+/−1.6%) body weight at Day 12. Pre-irradiation treatment animals lost 20.2% (+/−2.4%) body weight at Day 12, while post-irradiation treatment animals lost 10.6% (+/−1.9%) body weight at Day 12.

2. CBC (Blood Differentials):

Blood differentials evaluated white blood cell (WBC), lymphocyte (LYM), monocytes (MON), granulocyte (GRA), red blood cell (RBC), and platelet (PLT) levels. Results are reported as cells/liter and normalized to the non-irradiated control group values.

3. Six Hours

White blood cell and lymphocyte levels trended to decrease significantly in irradiated animals. Animals pre-treated with Homspera® were observed to have lower lymphocyte counts than test non-irradiated control (vehicle control) and irradiated control animals. Monocyte and granulocyte counts in irradiated animals were observed to increase significantly for both the irradiated control group and the Homspera® pre-treatment group. Monocytes increased about 200% over non-irradiated controls and granulocytes increased about 300% over non-irradiated controls. Red blood cell and platelet levels in irradiated animals were not found to differ significantly from non-irradiated controls.

4. 24 Hours Post Exposure

White blood cell counts in irradiated animals continued to be lower than non-irradiated controls at 24 hours post-exposure. Animals treated with Homspera® (both pre- and post-irradiation treatment) had white blood cell counts higher than that of the irradiated controls. Animals treated with Homspera® post-radiation exposure were observed to have greater white blood cell counts than animals treated with Homspera® prior to radiation exposure (about 30% versus about 19%). This same trend was observed in both lymphocyte (about 17% versus about 4%) and monocyte counts (about 119% versus about 95%). However, monocyte counts in Homspera®-treated animals were similar to those seen in non-irradiated control animals while irradiated control animals were observed to have a nearly 5 fold decrease in monocyte levels (about 100% versus about 20%). Granulocyte counts in animals treated with Homspera® post exposure were significantly greater (about 158%) than those observed in non-irradiated controls (100%) and animals exposed to Homspera® prior to radiation exposure (about 95%), while animals exposed to radiation and administered vehicle control were observed to have decrease granulocyte counts (about 75%). Again, red blood cell and platelet counts in irradiated animals were not observed to be significantly different from that seen in non-irradiated controls.

5. 48 hours post exposure

White blood cell counts in irradiated animals at 48 hours post-radiation exposure were very similar to those observed at 24 hours post-exposure. Again, irradiated controls were observed to have WBC counts lower than non-irradiated controls (about 17% versus 100%), while the post-radiation subjects had about double that amount (about 38%). Lymphocyte counts at 48 hours post-exposure mirrored those observed at 24 hours post-exposure. Animals treated post-radiation exposure had the highest lymphocyte levels (about 18%) of the three irradiated groups. Monocyte and granulocyte levels followed this trend as well. Animals treated with Homspera® post-radiation exposure were observed to have monocyte and granulocyte levels greater than that of non-irradiated animals, control irradiated animals and pre-radiation treatment animals (about 120% vs. 100%, 38% and 75% for monocytes; 170% vs 100%, 78% and 102% for granulocytes). Red blood cell counts were not significantly different for animals exposed to radiation. However, animals exposed to radiation were observed to have significantly reduced platelet counts. Animals exposed to Homspera®, either pre- or post-radiation exposure were observed to have platelet counts lower than that of control irradiated animals (about 40%).

6. 96 Hours Post Exposure

White blood cell counts continued to decrease at 96 hours post-exposure. At this time point, irradiated animals treated with vehicle or Homspera® pre-radiation exposure had white blood cell counts roughly 1/25^{th} that of non-irradiated controls (about 4%). Animals treated with Homspera® following radiation exposure had white blood cell counts roughly 3 times greater (about 12%) than those observed in irradiated control animals. Interestingly, similar results were observed for lymphocytes, monocytes, and granulocytes. Monocyte and granulocyte levels in irradiated animals fell dramatically at 96 hours post-exposure compared to 48 hour results. Again, animals treated with Homspera® following radiation exposure were observed to have substantially greater cell counts than those which were irradiated and treated with a vehicle control. Red blood cell counts continued to remain essentially unchanged. Platelet counts were observed to be similar to that seen at 48 hours post-radiation exposure. Nearly 50% reduction in platelet counts was observed in irradiated animals, and a small decreasing trend in Homspera® treated animals.

7. Flow Cytometry

Flow cytometry was conducted to identify and quantify cell markers in the animal groups but the results did not reveal conclusive trends. For example, non-irradiated control animals were observed to have a significant variance in positive CD34 cells and positive Sca-I cells over the 4 time points measured (6, 18, 24, and 48 hours). Non-irradiated controls were observed to have a decreasing trend in percent positive CD117 and CD9 cells. Similar results were observed in irradiated animals.

C. Discussion

A study was executed to evaluate the physiological effects of radiation and treatment with Homspera® (intranasally administered 25 μL of 300 μM solution) on mice. Mice were grouped into non-irradiated controls, irradiated controls, irradiated/treated pre-exposure, and irradiated/treated post-exposure. Irradiated animals were exposed to 4 Gy X-ray irradiation at a rate of 1/Gy per minute. Irradiated animals not treated with Homspera® were observed to have dramatic losses in body weight and significant decreases in CBC markers.

Animals treated with Homspera® for 8 days, beginning 1 day pre-radiation exposure, were observed to have weight losses greater than that of irradiated/non-treated animals. Alternatively, animals treated with Homspera® for 7 days following radiation exposure were observed to have a decreased weight reduction in comparison to irradiated controls.

Animals exposed to radiation were observed to have a significant reduction in white blood cell counts, lymphocyte counts, monocyte counts, granulocyte counts, and platelet counts. Most of these effects were observed as early as 6 hours post-radiation exposure; however decreases in platelet levels were not observed until 48 hours post-exposure. Treatment with Homspera® prior to radiation exposure (and 7 days thereafter) resulted in increases in white blood cell, lymphocyte, monocyte, and granulocyte counts when compared to irradiated controls. However, treatment with Homspera® for 7 days beginning after exposure to radiation was observed to have even greater effects on these same cell types. Animals treated with Homspera® following radiation exposure were observed to have greater monocyte and granulocyte counts than non-irradiated control animals for the first 48 hours following radiation exposure. However, these effects were not seen at 96 hours post-exposure, as monocyte and granulocyte counts were dramatically reduced.

Analysis of flow cytometry data did not reveal conclusive trends for any of the groups tested. Non-irradiated control animals were observed to have a level of variance similar to that seen in irradiated/drug-treated animals. This may be a product of biological variance, equipment variance, or both.

Animals exposed to radiation were observed to have a dramatic decrease in weight that continued until sacrifice or death. Animals treated with Homspera® daily for 7 days following irradiation were observed to have significantly less weight loss. The post-irradiation treatment group was also observed to have significantly increased levels of white blood cells, lymphocytes, monocytes and granulocytes when compared to irradiated control animals and pre-irradiation treatment animals. Platelet levels were observed to decrease significantly in all irradiated animals after 48 hours post-exposure. Treatment with Homspera® for 7 days following radiation exposure yielded the most efficacious method of maintaining animal weights and increasing vital CBC markers.

### Example 3

### The Effect of an Exemplary Substance P Analog, Homspera®, on Cellular Differentiation and Proliferation

A. Introduction

In the study described below, human bone marrow-derived hematopoietic cell populations (or hematopoietic stem cells, HSCs) were cultured with or without Homspera® to determine whether Homspera® affects proliferation or differentiation of the cells.

To assess proliferation, intracellular ATP (iATP) levels were measured. Increased levels of iATP correlate with increased cellular proliferation, because cells that are proliferating typically require high levels of energy, which is provided by iATP.

To examine or assess proliferation, in this case, the experiment was designed to compare the effects of Homspera® on proliferation and differentiation after a 14 day incubation period. Although more or increased concentrations of cytokines or growth factors are typically added to support differentiation than proliferation alone, these concentrations are still effective for proliferation. Therefore proliferation was examined under the same conditions as those used for differentiation

To induce differentiation, “optimal” concentrations of growth factors and cytokines were used. Rich, 2003, Curr. Op. Drug Discovery Devel. 6:100-109, Rich and Hall, 2005, J. Tox. Sci. 87(2): 427-441. However, because substance P is known to stimulate hematopoiesis and promote the release of cytokines that contribute to differentiation, “suboptimal” concentrations of growth factors and cytokines were also used in the event the effects of Homspera® would not be observed under saturating and therefore optimal cytokine conditions. Suboptimal concentrations were approximately one-fifth of optimal concentrations and were concentrations known to support colony formation. Rich, personal communication.

B. Methods

Homspera® (5 mg, Lot E844) was shipped as a solid compound. The compound was dissolved in 1 ml of Iscove's Modified Dulbecco's Medium (IMDM) and a serial dose response prepared in single log doses so that the final dose in culture ranged from 1 nM to 1×10⁻¹⁶ M. All working dilutions were performed in IMDM.

Starting with human bone marrow aspirate, the mononuclear cell (MNC) fraction was separated from the whole bone marrow using Ficoll-Paque density gradient centrifugation. The resulting MNC fraction had a cell concentration of 6.2×10⁶ cells/ml with a viability of 99.9%. The cell concentration was adjusted so that the final cell concentration in culture was 10,000 cells/well.

Human bone marrow MNC was dissolved in IMDM and cultured at a concentration of 10,000 cell/well in a CAMEO™-96 Master Mix (HemoGenix, Inc., Colorado Springs, Colo.). Master Mix is a HemoGenix proprietary cell culture media comprised of a serum mix (4 parts), a methyl cellulose mix (4 parts) and a growth factor mix (1 part) with the bone marrow target cells (1 part). See, Rich and Hall 2005, Toxicol. Sci. 87(2): 427-441.

Different combinations and concentrations of growth factors are used to induce differentiation of cells into specific cell types. The target cell populations were: High Proliferative Potential-Stem and Progenitor cell (HPP-SP), Colony-forming Cells-Granulocyte, Erythroid, Macrophage, Megakaryocyte (CFC-GEMM), Blast Forming Unit-Erythroid (BFU-E), Granulocyte-Macrophage-Colony Forming Cells (GM-CFC), Megakaryocyte-Colony Forming Cells (Mk-CFC), T-lymphocyte-Colony Forming Cells (T-CFC), B-lymphocyte-Colony Forming Cells (B-CFC).

Growth factors or cytokines used in the study were: erythropoietin (EPO), granulocyte macrophage-colony stimulating factor (GM-CSF), granulocyte-colony stimulating factor (G-CSF), Interleukins 3, 6, 2 and 7 (IL-3, IL-6, IL-2 and IL-7), stem cell factor (SCF), thrombopoietin (TPO) and soluble mutant flt3 ligand (Flt3-L).

The concentrations of growth factors or cytokines used for each cell type assay are provided in Table 4 (Optimal Growth Concentrations) and Table 5 (Sub-Optimal Growth Concentrations).

TABLE 4
Optimal Growth Factor or Cytokine Concentrations for 7 Cell Populations (/ml)

| | EPO | GM-CSF | G-CSF | IL-3 | IL-6 | SCF | TPO | Flt3-L | IL-2 | IL-7 |
|---|---|---|---|---|---|---|---|---|---|---|
| HPP-SP | 3 U | 20 ng | 20 ng | 10 ng | 20 ng | 50 ng | 50 ng | 50 ng | 50 ng | 40 ng |
| CFC-GEMM | 3 U | 20 ng | 20 ng | 10 ng | 20 ng | 50 ng | 50 ng | 50 ng | — | — |
| BFU-E | 3 U | — | — | 10 ng | — | 50 ng | — | — | — | — |
| GM-CFC | — | 20 ng | — | 10 ng | — | 50 ng | — | — | — | — |
| Mk-CFC | — | — | — | 10 ng | — | 50 ng | 50 ng | — | — | — |
| T-CFC | — | — | — | — | — | — | — | — | 50 ng | — |
| B-CFC | — | — | — | — | — | — | — | — | — | 40 ng |

TABLE 5
Sub-Optimal Growth Factor or Cytokine Concentrations for 7 Cell Populations (/ml)

| | EPO | GM-CSF | G-CSF | IL-3 | IL-6 | SCF | TPO | Flt3-L | IL-2 | IL-7 |
|---|---|---|---|---|---|---|---|---|---|---|
| HPP-SP | 0.06 U | 0.4 ng | 0.4 ng | 0.2 ng | 0.4 ng | 1 ng | 1 ng | 1 ng | 1 ng | 0.8 ng |
| CFC-GEMM | 0.06 U | 0.4 ng | 0.4 ng | 0.2 ng | 0.4 ng | 1 ng | 1 ng | 1 ng | — | — |
| BFU-E | 0.06 U | — | — | 0.2 ng | — | 1 ng | — | — | — | — |
| GM-CFC | — | 0.4 ng | — | 0.2 ng | — | 1 ng | — | — | — | — |
| Mk-CFC | — | — | — | 0.2 ng | — | 1 ng | 1 ng | — | — | — |
| T-CFC | — | — | — | — | — | — | — | — | 1 ng | — |
| B-CFC | — | — | — | — | — | — | — | — | — | 0.8 ng |

U = Unit, ng = nanogram

The sub-optimal concentrations were about 50 fold less than the optimal concentrations used.

Eleven μl of the diluted Homspera® solution was added to each well followed by 100 μl of the master mix for each cell population detected. The cells were incubated in the absence or presence of Homspera® under sub-optimal and optimal stimulatory conditions for 7 target cell populations in 96-well plates for 14 days at 37° C. in a fully humidified atmosphere comprised of 5% CO₂ and 5% O₂. The total colony counts were manually enumerated under an inverted microscope, followed directly by processing the plates for bioluminescence to determine the intracellular ATP concentrations of the cells in each well. The study was concluded within 30 days of obtaining Homspera® and within 14 days of obtaining the human bone marrow aspirate.

Prior to processing all 96-well plates, the total colony counts/well were manually enumerated by microscopy. The mean, standard deviation and percent coefficient of variation was calculated for all groups, transposed and plotted. The intracellular ATP (iATP) concentration was measured after manual enumeration.

The output of the luminometer is non-standardized Relative Luminescence Units (RLU). Prior to measuring the samples, an ATP standard curve was performed. This allowed the RLU values to be automatically calculated into standardized ATP (μM) units. For both the RLU and ATP values derived from each well, the luminometer software calculated the mean, standard deviation and percent coefficient of variation.

C. Results

The data indicate Homspera® stimulates proliferation or differentiation of hematopoietic stem cells (HSCs) isolated from human bone marrow MNCs. Homspera® stimulated proliferation as indicated by the increase in iATP. Homspera® also stimulated differentiation as indicated by the increased colony forming units of hematopoietic progenitor cells.

Some background is helpful to understanding the results. A traditional CFC assay is usually performed in duplicate in 35 mm Petri dishes. Sub-optimal growth factor studies cannot be performed with commercial media. Instead, the individual reagents have to be prepared and added individually. In this study, optimum growth factor/cytokine concentrations would be those that are normally used for the CFC assay. This assay is a functional differentiation assay, meaning that the assay relies on the functional ability of the target cells to divide and differentiate into colonies containing cells that identify the types of colonies being produced. Many lineage-specific growth factors, e.g. EPO, GM-CSF, TPO, are known to exhibit bifunctionality in that they will act as a proliferation factor for primitive cells in the series, but as a survival factor for the differentiating and maturating cells. Without the factors, the cells will enter into apoptosis. To induce proliferation, lower concentrations of growth factors or cytokines are required. These concentrations will, in many cases, be sub-optimal for the CFC differentiation assay. If proliferation only had been measured using the ATP assay at 7 days rather than 14 days, it is possible that a different response might have been observed using optimal and sub-optimal growth factor/cytokine concentrations. For the 7 cell populations detected in this study, the growth curve at 14 days indicates that proliferation decreased as differentiation increased. Therefore, the iATP concentration detected at 14 days represents residual proliferation within the colonies. However, since both the CFC and ATP assays were performed under the same conditions, it is possible to directly compare the results from the two separate readouts. Taking these factors into account, the response between the 7 cell populations is probably best described as a percentage of the respective control.

1. Controls

The control values after 14 days in culture are shown in Table 6 and Table 7. In most cases, the control results are within the expected range of values with the cell populations falling into three categories: stem cell populations, myelopoietic populations and lymphopoietic populations. The 2 stem cell populations (HPP-SP and BFU-E) show the greatest proliferation and differentiation potential followed by the 3 myelopoietic populations (CFC-GEMM, GM-CFC and Mk-CFC) and the 2 lymphopoietic populations (T-CFC and B-CFC).

TABLE 6
iATP Proliferation Assay Controls

| | Optimal Conditions | | Sub-Optimal Conditions | |
|---|---|---|---|---|
| | Mean | Std. Dev. | Mean | Std. Dev. |
| Background | 0.213 | 0.021 | 0.320 | 0.026 |
| HPP-SP | 0.591 | 0.088 | 0.802 | 0.254 |
| CFC-GEMM | 0.31 | 0.078 | 0.815 | 0.157 |
| BFU-E | 0.664 | 0.077 | 0.781 | 0.199 |
| GM-CFC | 0.525 | 0.144 | 0.914 | 0.03 |
| Mk-CFC | 0.483 | 0.057 | 0.655 | 0.22 |
| T-CFC | 1.165 | 0.208 | 0.649 | 0.158 |
| B-CFC | 0.691 | 0.105 | 0.417 | 0.109 |

TABLE 7
CFC Differentiation Assay Controls

| | Optimal Conditions | | Sub-Optimal Conditions | |
|---|---|---|---|---|
| | Mean | Std. Dev. | Mean | Std. Dev. |
| Background | 6.8 | 1.0 | 16.8 | 3.9 |
| HPP-SP | 109.0 | 7.8 | 74.7 | 12.4 |
| CFC-GEMM | 94.0 | 7.4 | 56.8 | 5.0 |
| BFU-E | 114.8 | 168.1 | 24.5 | 10.4 |
| GM-CFC | 53.7 | 4.1 | 50.7 | 9.5 |
| Mk-CFC | 38.7 | 12.2 | 38.2 | 7.2 |
| T-CFC | 38.5 | 6.3 | 15.0 | 1.8 |
| B-CFC | 22.8 | 2.1 | 17.5 | 3.0 |

2. Effect of Homspera® on Differentiation

In the presence of optimal growth factors, all cell populations, with the exception of BFU-E, exhibited enhancement or potentiation in differentiation potential. The response of BFU-E was significantly lower than the controls at all compound doses, although a slight increase from the lowest dose at 10⁻¹⁶M to 10⁻¹³M was observed prior to a decrease to the highest dose used (1 nanoMolar (nM)). For B-CFC, the dose response was a bell-shaped curve, beginning below control values at 10⁻¹⁶M, but peaking at 1 picomolar (pM) at about 163%, prior to decrease to control values. The Mk-CFC and CFC-GEMM populations also increased from control values at the lowest dose to reach peak between 10⁻¹⁴M and 10⁻¹³M respectively before decrease to control values at the highest dose of Homspera®. HPP-SP, T-CFC and GM-CFC all started at values significantly higher than control at the lowest dose. The T-CFC produced an approx. plateau between 10⁻¹⁶M and 10⁻¹³M before decreasing to control levels. The HPP-SP population peaked at 10⁻¹⁵M and decreased thereafter to control values. The GM-CFC produced the greatest potentiation of all cell populations peaking at 10⁻¹⁴M.

Under sub-optimal growth factor/cytokine conditions, both GM-CFC and CFC-GEMM produced a dose response that peaked at 10⁻¹⁴M and decreased thereafter, although at the highest dose of 1 nM, the values from these two population did not fall below control values. In contrast, the B-CFC and HPP-SP populations, produced a very gradual increase with a slight decrease at 1 nM. The T-CFC hovered around control values and exhibited a decrease to below control values after 10⁻¹³M. Both the BFU-E and Mk-CFC were below control values for essentially the whole dose response, although a peak did occur at 10⁻¹⁴M for Mk-CFC and 10⁻¹³M for BFU-E. For those populations that exhibited values greater than control at the lowest dose used, the dose response could be extended to doses lower than 10⁻¹⁶M.

3. Effect of Homspera® on Proliferation

The ATP proliferation assay shows a different profile to that of the differentiation assay for all cell populations. Like the CFC assay at optimum growth factor/cytokine conditions, BFU-E exhibited a dose response below control values, with a gradual increase to control values at the highest dose used. The dose response for T-CFC was essentially flat at control levels. The B-CFC exhibited a flat dose response over the complete dose range, but at approx. 200% of control values. All other populations, (HPP-SP, CFC-GEMM, GM-CFC and Mk-CFC) exhibited an unusual U-shaped dose response curve, decreasing from the lowest Homspera® dose to about 10⁻¹⁴M and increasing again from about 10 fM to 1 nM.

At sub-optimal growth factor/cytokine concentrations, only the lymphopoietic cell populations (T-CFC and B-CFC) exhibited a potentiation between 200 and 300% above control values. However, for both of these cell populations, the dose response was essentially flat. The Mk-CFC population exhibited essentially no response, while HPP-SP, CFC-GEMM, BFU-E and GM-CFC exhibited dose responses that were below control levels for most of the doses used.

However, although B-CFC are enhanced under optimal and sub-optimal condition in the ATP proliferation and T-CFC are enhanced under sub-optimal conditions also in the ATP proliferation assay, this enhancement effect is not dose-dependent, at least over the dose range used. The absence of a dose response indicates that the response observed may actually be a plateau effect and that the cell populations are sensitive to the compound at much lower doses than were tested in this study. In addition, these cells do not demonstrate toxicity at the levels tested.

D. Discussion

After 14 days of incubation, Homspera® exhibited its maximum effect on the differentiation, rather than the proliferation process. Notable, for most of populations, was the apparent absence of distinct cytotoxicity. For both the CFC differentiation and ATP proliferation assays, the BFU-E population was the only population that was suppressed under optimal and sub-optimal conditions. However, see Example 4, below, where BFU-E differentiation and/or proliferation was enhanced.

The GM-CFC exhibited the greatest enhancement in the CFC differentiation assay, a result which is in accordance with published data for substance P. The T-CFC and B-CFC exhibit a dose response in the CFC differentiation assay under optimal and sub-optimal conditions. However, although B-CFC are enhanced under optimal and sub-optimal condition in the ATP proliferation and T-CFC are enhanced under sub-optimal conditions also in the ATP proliferation assay, this enhancement effect is not dose-dependent, at least over the dose range used. The absence of a dose response indicates that the response observed may actually be a plateau effect and that the cell populations are sensitive to the compound at much lower doses than were tested in this study. In addition, these cells do not demonstrate toxicity at the levels tested.

Several of the effects observed using the CFC differentiation readout have also been found for substance P and published in the literature. The difference between results of the ATP proliferation assay and the CFC differentiation assay is noteworthy. Firstly, higher doses of Homspera® enhance differentiation rather than proliferation, an effect known for lineage-specific growth factors, for example, erythropoietin. Secondly, for most lympho-hematopoietic cell populations exposed to the present dose range, the primary effect of Homspera® is during differentiation or maturation.

For those populations that exhibited values greater than control at the lowest dose used, the dose response could be extended to doses lower than 10⁻¹⁶M. In evaluating the colony numbers prior to detecting iATP, it did appear that the change in colony numbers was due to a change in the size of the colonies as the compound dose increased. This was particularly the case for the HPP-SP population.

Homspera® was effective at stimulating differentiation of several hematopoietic progenitor cells under both optimal and sub-optimal growth factor conditions. Under sub-optimal conditions HPP-SP, GM-CFC, CFC-GEMM and B-CFC were noticeably stimulated to differentiate. Under optimal conditions, HPP-SP, GM-CFC, T-CFC, Mk-CFC, CFC-GEMM and B-CFC progenitor cells were stimulated to differentiate.

Granulocyte/macrophage progenitors were the most responsive to Homspera® and were stimulated approximately 250% and 200% above controls lacking Homspera® treatment for both optimal and sub-optimal growth factor conditions respectively. CFC-GEMM cells were also stimulated to surprising levels above controls at about 175% in optimal conditions and about 225% in sub-optimal conditions. HPP-SP and T-CFC cell numbers were both about 175% above control values for optimal conditions. Under sub-optimal conditions, T-CFC populations did not change much from control values, whereas HPP-SP populations were enhanced roughly 125% from the population controls. In optimal conditions, B-CFC was 160% above control populations at Homspera® concentration of about 10⁻¹²M. The effects of Homspera® on B-CFC cells was not as pronounced under sub-optimal conditions, and were only stimulated 125% from the control population.

Homspera® was also effective at stimulating proliferation as measured by iATP levels using a fluorescent read-out. The most notable effects of Homspera® were on B-CFC and T-CFC progenitors cultured under sub-optimal cytokine levels. B-CFC iATP levels increased nearly 300% from control populations lacking Homspera® and T-CFC iATP levels increased 200% from controls. Furthermore Homspera® is effective at the lowest dose tested, 10⁻¹⁶M, suggesting biological activity for proliferation at sub-femtomolar concentrations. The results for the optimal growth factor conditions are similar to the differentiation assays in that the same cell types were stimulated with Homspera® (HPP-SP, GM-CFC, T-CFC, Mk-CFC, CFC-GEMM and B-CFC progenitor cells). B-CFC iATP levels were again significantly higher than controls (200%).

### Example 4

### The Effect of an Exemplary Substance P Analog, Homspera®, on Cellular Differentiation and Proliferation

### Multi-Donor Study

The study was undertaken to further illustrate lympho-hematopoietic differentiation in response to Homspera® using human-derived hematopoietic cell populations from three different bone marrow donors.

Homspera® (5 mg, Lot F209) was shipped as a solid compound and stored at 4° C. upon arrival. Compound was dissolved in 1 ml of Iscove's Modified Dulbecco's Medium (IMDM) and a serial dose response was prepared in single log doses so that the final dose in culture ranged from 1 nanoMolar (nM) to 1×10⁻¹⁶ M. All working dilutions were performed in IMDM. Cell cultures from each donor were started at different times and a fresh solution of Homspera® was prepared for each individual experiment.

Starting with human bone marrow aspirate, the mononuclear cell (MNC) fraction from each aspirate was separated from whole bone marrow using Ficoll-Paque density gradient centrifugation.

The colony-forming cell (CFC) assay was performed using optimal growth factor/cytokine concentrations. The reagents and conditions were similar to those used in Example 3, except that no ATP measurements were performed. The MNC fraction from each bone marrow donor was dissolved in IMDM and cultured at a concentration of 5,000 cells/well in a Culture Master Mix (HemoGenix, Inc., Colorado Springs, Colo.).

The target cell populations were: High Proliferative Potential-Stem and Progenitor cell (HPP-SP), Colony-Forming Cells-Granulocyte, Erythroid, Macrophage, Megakaryocyte (CFC-GEMM), Blast Forming Unit-Erythroid (BFU-E), Granulocyte-Macrophage-Colony Forming Cells (GM-CFC), Megakaryocyte-Colony Forming Cells (Mk-CFC), T-lymphocyte-Colony Forming Cells (T-CFC), B-lymphocyte-Colony Forming Cells (B-CFC).

Growth factors or cytokines used in the study were: erythropoietin (EPO), granulocyte macrophage-colony stimulating factor (GM-CSF), granulocyte-colony stimulating factor (G-CSF), Interleukins 3, 6, 2 and 7 (IL-3, IL-6, IL-2 and IL-7), stem cell factor (SCF), thrombopoietin (TPO) and soluble mutant Flat 3 ligand (Flt3-L).

The concentrations of growth factors or cytokines used for the CFC assay are the same as those provided in Table 4 of Example 3, e.g. Optimal conditions.

The assay was performed in a 96-well plate. To each well, 11 μl of the test compound (Homspera®) dilution was added followed by 100 μl of the Culture Master Mix for each cell population detected. Cultures were incubated for 14 days at 37° C. in a fully humidified atmosphere containing 5% CO₂ and 5% O₂. Thereafter, the total colony counts were manually enumerated under an inverted microscope.

The mean, standard deviation and percent coefficient of variation was calculated for all groups, transposed and plotted as a function of donor. The percent from control values were calculated and also plotted as a function of donor. In addition, results were compared from each individual cell population from all donors. All results were plotted using Prism Version 5 for Mac.

A. Results: Response from Individual Cell Populations

1. Stem Cells:

For all three donors, the primitive HPP-SP stem cells exhibited a greater response than the more mature multi-potential CFC-GEMM stem cells. Variations with respect to the level of potentiation did occur; and at the highest doses, a decrease in colony counts was usually observed. For example, with the HPP-SP cells, donor 1 was stimulated to approx. 300% from controls at Homspera® concentrations of 10⁻¹² M and 10⁻¹¹ M, while donors 2 and 3 were approx. 200% and 175% from controls respectively. Both donors 2 and 3 were maximally stimulated at 10⁻¹⁵ M Homspera®, a concentration lower than that for donor 1.

The variation between donors for CFC-GEMM was less pronounced and the trend was similar to levels previously observed with optimal growth factors in Example 3. Donor 1 exhibited the greatest potentiation to greater than 200% from controls, while donors 2 and 3 were both around 175% from controls. For all three donors, the maximum effects of Homspera® were observed at the 10⁻¹⁴ M dose.

2. Hematopoietic Lineage Cells:

For the GM-CFC population, donors 1 and 2 demonstrated responses above background, while donor 3 was very close to control levels for nearly all the Homspera® concentrations tested. Both donors 1 and 2 were maximally stimulated to 200% and 150% relative to controls at 10⁻¹³ M Homspera®, respectively. The trend observed for GM-CFC is similar to that from the single marrow donor study. Additionally, the two-fold enhancement of colony forming activity and effectiveness at low Homspera® concentrations is consistent with the multi-donor study of Example 6.

This study showed that for all three donors, BFU-E exhibited a response that was in most cases significantly greater than the control over the entire Homspera® dose range. Indeed BFU-E showed the greatest response of all cell populations from the second donor at 10⁻¹¹ M Homspera®. Colony numbers were enhanced to approx. 150-300% relative to controls, depending on the donor. These levels of stimulation are consistent with the multi-donor study of Example 6, examining the effects of Homspera® on BFU-E colony formation.

The Mk-CFC population also exhibited a varied dose response to Homspera®. However, whereas donor 1 demonstrated an overall increase in potentiation with increasing compound dose, the response of donors 2 and 3 was relatively flat, and that from donor 3 was either below or near control levels over the Homspera® doses examined. While the concentration for maximal stimulation varies between donors, donors 1 and 2 both stimulated megakaryocyte colony formation greater than 200% from controls.

3. Lymphopoietic Cell Populations:

These two populations demonstrated the greatest effects of Homspera® with donor 1. The T-CFC response was greater than 300% from controls and greater than 200% for B-CFC for donor 1. However, the maximum effect of Homspera® on donors 2 and 3 was less than 150% from controls. T-CFC for donors 2 and 3 demonstrated a gradual increase with increasing compound dose, but the B-CFC demonstrated a slight decrease with increasing compound dose. The T-CFC population for Donors 2 and 3 exhibited a maximum at 0.1 picoMolar (10⁻¹²M), slightly above control values. For Donors 2 and 3, the peak value occurred at 1×10⁻¹⁵M.

B. Conclusions

The overall response of the three donors was that Homspera® potentiates differentiation of all seven cell populations tested when stimulated with optimal growth factor and cytokine concentrations. There was no apparent toxicity at the highest doses.

Homspera® is effective at increasing colony formation of the multi-lineage progenitor CFC-GEMM, and consistently acts to stimulate the colonies produced from CFC-GEMM.

### Example 5

### Effect of an Exemplary Substance P Analog, Homspera®, on Stem Cell Proliferation and Differentiation

The purpose of the study was to determine if Homspera® (ImmuneRegen BioSciences, affects primitive lympho-hematopoietic stem cell (cells of both the lymphoid & myeloid lineages) proliferation and differentiation as detected using HALO®-96 HPPr (HemoGenix) and phenotypic analysis.

A. Materials and Methods

Homspera® (Lot F209) was shipped as a solid compound. The compound was dissolved in Iscove's Modified Dulbecco's Medium (IMDM) and a serial dose response was prepared in single log doses so that the final dose in culture ranged from 1 nanoMolar (nM) to 1×10⁻¹⁶ M. All working dilutions were performed in IMDM.

Human bone marrow cells were obtained. The mononuclear cell (MNC) fraction was separated from whole bone marrow using Ficoll-Paque density gradient centrifugation. The sample contained 9.38×10⁶ cells/ml, the total nucleated cell (TNC) count was 2.81×10⁷, with viability of 99.1%.

The HALO®-96 HPPr assay (HemoGenix, Inc., Colorado Springs, Colo.) is a primary and secondary re-plating assay for the primitive lympho-hematopoietic stem cell population termed high proliferative phase-stem and progenitor cells (HPP-SP). U.S. Pat. Nos. 7,354,729, 7,354,730 and U.S. patent application Ser. No. 11/561,133, incorporated by reference herein in their entireties.

The HPP-SP cell population is normally quiescent, i.e. in G₀ of the cell cycle. The first step of the assay involved preparing cultures in which the HPP-SP are “primed” out of quiescence and into the cell cycle using a growth factor/cytokine cocktail containing interleukin-3 (IL-3), interleukin-6 (IL-6), stem cell factor (SCF), thrombopoietin (TPO) and “flat” 3 ligand (Fl3-Ligand) (referred to as “primary assay”). The MNC were cultured under Suspension Expansion Culture (SEC) conditions at a concentration of 10,000 cells/well for 5 days at 37° C. in a fully humidified atmosphere containing 5% CO₂ and 5% O₂.

Two treatment groups and two controls were created. Treatment group 1 having Homspera® alone without the “priming” cocktail and Treatment group 2 having Homspera® with the “priming” cocktail. Two control groups were created. Control group 1 being background, no growth factors added to cells and Control 2 having cells stimulated with “priming” cocktail.

For each treatment, 24 replicate wells were prepared for each of the eight Homspera® doses tested, from 1×10⁻¹⁶M to 1×10⁻⁹M in log doses. After five days of incubation, eight replicate wells from each dose and from each treatment were used to measure proliferation of the cells by detecting intracellular adenosine triphosphate (iATP) by bioluminescence. The cells in the remaining 24 wells from each dose treatment and controls were removed and pooled. The cells were centrifuged, the supernatant discarded and a cell concentration determined for each treatment and control. The total cell counts indicated that there was no effect of Homspera® alone on the cell count, however, in the presence of Homspera®+priming cocktail, an increase in cell count above that obtained for the control was observed. The greatest cell count was found with a Homspera® dose at 0.1 picomolar (pM).

The cells were then prepared for the secondary re-plating step (“secondary assay”) by preparing SEC cultures for each dose treatment and control in which cultures were subjected to “full stimulation”. Each culture contained 4,000 cells/well and 12 replicates were prepared in 96-well plates. The cultures were incubated under the same conditions as in the primary assay, but the incubation period was extended to six days. At this time point, eight replicates were used to determine the iATP concentrations and therefore proliferation. The cells in the remaining eight replicates wells from each treatment dose and control were removed, pooled and centrifuged. After discarding the supernatant, the cells were resuspended in approx. 0.7 ml of IMDM. Insufficient cells were available to perform a cell count on each tube. The cell suspension was then divided into six tubes which were then stained with different panels of fluorochrome-conjugated antibodies to various cluster of differentiation (CD) markers.

The panels used were as follows: Panel 1 CD90/CD133/CD45/CD34; Panel 2 CD61/CD41/CD45; Panel 3 CD45/CD15/CD14; Panel 4 CD45/Glycophorin-A/7-AAD; Panel 5 CD45/CD38/CD117 and Panel 6 CD45/CD56/CD19/CD3. Included in all panels was the CD45 pan-leukocyte marker, present on all hematopoietic cells. See also, Table 8.

A maximum of 5,000 events were acquired by flow cytometry and the results were evaluated using FloJo version 8.7 software for Mac. To exclude non-hematopoietic cells from the analyses, the presence (or absence) of membrane expression markers was “gated” through the CD45 “gated” region. Thus, if a population is CD34⁺ or CD34⁺/CD133⁺, both are also CD45⁺. Similarly, if a population is Glycophorin-A⁺, it is also CD45⁺. This gating procedure can exclude some very primitive hematopoietic stem cells that may be CD45⁻, but also excluded mesenchymal stem cells which are known to be CD45⁻. All results are therefore depicted as percent from CD45⁺ cells, rather than the total population.

TABLE 8
Stem Cell Expression Markers

| Cell Membrane Expression | |
|---|---|
| Marker | Remarks |
| CD34 | Present on stem cells and primitive |
| | hematopoietic progenitor cells |
| CD90 | Also known as mouse Thy1 antigen: present |
| | on primitive stem cells |
| CD133 | primitive stem cell marker, although known |
| | to be present on other cell types |
| CD61 | integrin β3 subunit; associates with CD41 |
| | (integrin α2b subunit), to form the |
| | heterodimeric gpIIb/gpIIIa present on |
| | megakaryocytes and platelets |
| CD41 | integrin α2b subunit; associates with CD61 |
| | (integrin β3 subunit), to form the |
| | heterodimeric gpIIb/gpIIIa present on |
| | megakaryocytes and platelets |
| CD14 | Monocytes and macrophages |
| CD15 | Granulocytes |
| Glycophorin-A (CD235a) | Erythroid cells |
| 7-Aminoactinomycin D | Used to detect cell viability |
| (7-AAD) | |
| CD38 | Present on most early committed |
| | hematopoietic cells |
| CD117 | c-kit/stem cell antigen; also present on |
| | non-hematopoietic cells |
| CD3 | T-lymphocyte marker |
| CD19 | B-lymphocyte marker |
| CD56 | Natural killer (NK) cells |

B. Results: Effects of Homspera® on Stem Cell Proliferation

Two treatment groups and two control groups were tested for the ability to proliferate HPP-SP cells. Homspera® was tested in the absence of a priming cocktail to determine if Homspera® alone has the ability to “prime” the quiescent HPP-SP cells out of phase G₀ and into the cell cycle (i.e. primary assay). Additionally, Homspera® was examined with the priming cocktail to determine if Homspera® modifies the proliferative potential of the “primed” HPP-SP cells (i.e. secondary assay).

Human MNC-derived primitive HPP-SP stem cells were primed out of quiescence over a five day incubation period in the presence of increasing doses of Homspera® alone. Under these conditions, there was no dose-dependent increase or decrease in proliferation as detected by ATP-based bioluminescence. Additionally, Homspera® alone was not capable of substituting for the growth factors and cytokines used in the priming mix as demonstrated by the lack of ATP production in the primary assay with Homspera® alone compared to the primary assay of HPP-SP cells incubated with the “priming” cocktail.

Moreover, when Homspera® was tested in the presence of the “priming” cocktail, there was no dose-dependent effect on HPP-SP proliferation.

In the second part of the experiment, cells from all four treatment conditions were removed from the plates, washed and re-plated all in the absence of Homspera®, but with a growth factor cocktail that would promote “full stimulation” of the cells. Proliferation was measured using ATP bioluminescence after incubating the cells for six additional days at 37° C. in a fully humidified environment with 5% CO₂ and 5% O₂.

Under “full stimulation” using the cultures previously treated with Homspera® alone (secondary assay-Homspera® alone+“full stimulation”) or in the presence of Homspera®+“priming” cocktail (secondary assay-Homspera®+“full stimulation”), no definitive changes in proliferation were observed relative to the secondary assay background and secondary assay HPP-SP “full stimulation” controls. Therefore, no dose-dependent effects of Homspera® were observed.

To examine the effects of Homspera® on cell phenotype, cells were removed from the secondary re-plating after a six day incubation. Homspera®'s effects during the primary culture on both stem cells and differentiated cells after the secondary re-plating under “full stimulation” conditions were determined using flow cytometry.

Because all hematopoietic cells express the CD45+ marker, cells were gated for this marker and then further isolated based on the presence of additional CD markers depending on the phenotype of interest. To examine the effects of Homspera® on primitive hematopoietic stem and progenitor cells, antibodies were used against CD34+ cells, CD90+ cells, and CD133+ cell populations. The proportion of cells affected is small, which would be expected from the proportion of stem cells available in the CD45+ region. The effect of Homspera® alone on CD34+, CD90+ and CD133+ cells produces an increase in all 3 cell populations with a maximum at 0.01 pM (1×10⁻¹⁴M) followed by a decrease and an apparent plateau. At maximal stimulation, approximately 1% of the CD45+ cells have the markers from the 3 cell populations. In the presence of Homspera®+prime, there appears to be a gradual increase in CD34+ cells to 1 pM (about 0.5% of CD45+ cells) of Homspera®, followed by a decrease in cells expressing CD34+ as Homspera® concentrations increase. The proportion of CD133+ cells appears to decrease with increasing concentrations of Homspera® from about 0.75% of CD45+ cells to about 0.4%, while, with the exception of an apparent outlier at 0.01 pM, the percent of CD90+ cells increases to almost 2% of the CD45+ cells reaching a maximum at 0.1 pM, followed by a decrease in CD90+ cells to about 0.75% of CD45+ cells. The responses for all populations are greater than controls, with the CD90+ population being significantly increased in the Homspera®+prime treatment group. The increase in response of the stem cell populations to Homspera® alone would imply that a greater number of committed differentiated cells might become available when the cells are affected by growth factors and cytokines. The small increase in CD34+ cells in the presence of Homspera®+prime would imply a similar response. However, if CD133+ cells are more primitive than either CD90+ or CD34+ cells, then a decrease in this population would result in an increase in both CD90+ and CD34+ cells. This might be the reason for the slight increase in CD34+ and the more significant increase in CD90+ cells to Homspera®+prime. The increase in CD90+ cells in response to Homspera®+prime, might also be the precursor to the increase in CD3+ T-lymphocytes.

Next double positive stem cell populations expressing CD45+ were examined, including CD34+/CD133+, CD34+/CD90+, and CD90+/CD133+ cells. The double positive stem cell populations follow a similar pattern to the single positive stem cell response for HSP alone, producing a peak response at 0.01 pM. Note that the proportion of cells affected is approximately the same (ranging from about 0.65% to about 0.9% of CD45+ cells) as for the single positive cells which were around 1% of CD45+. However, the response of these double populations to Homspera®+prime is decreased compared to the single positive populations (values for double positive are all below 0.4% of CD45+ cells). Notably, CD34+/CD90+ and CD90+/CD133+ are both dramatically decreased compared to the single CD90+ population. The lower proportion of double positive cells might imply an efflux of primitive cells into the committed differentiation pathways. Compared to controls, the response to Homspera® alone is significantly increased. Homspera® has been shown to enhance formation of cell colonies in a differentiation assay from early stage stem cells, which likely includes these double positive populations.

To determine the effects of Homspera® on megakaryocytes, cells expressing the single markers CD41+ or CD61+, or cells expressing the double markers (CD41+/CD61+) were examined. Both CD61 and CD41 are sometimes used alone to determine the presence of megakaryocytes and platelets. The combination of these two definitively defines their presence. Therefore, with Homspera® alone, a low proportion of cells express the CD61+ antigen (about 2% of CD45+ cells), but there are very few CD41+ and double positive cells present (less than 0.5%). In contrast, in the presence of Homspera®+prime, both single positive and the CD41/CD61 double positive populations peak at 10 pM. The proportion of CD41+ and CD61+ single positive cells at this Homspera® dose is about 18% of the CD45+ cells while about 35% of the CD45+ cells express both CD41 and CD61 markers (double positive cells). This is a dramatic increase compared to the controls and implies that Homspera® may be particularly effective for this lineage. Furthermore, when bone marrow stem cells are exposed to growth factors and cytokines that promote formation of megakaryocyte colonies, addition of Homspera® to the cultures enhances platelet colony formation. In multi-donor experiments, Homspera® stimulates megakaryocyte colony forming activity at least two-fold above controls that are cultured in the absence of Homspera®. These combined experiments suggest that Homspera® can function at both early and late stages of stem cell maturation into megakaryocytes. The early functions of Homspera® are demonstrated by the increase in platelet phenotype seen from the Homspera®+prime samples. In this study, Homspera® was added only to the initial cultures which were being “primed” out of quiescence. The late-stage function for Homspera® is demonstrated by the colony forming assay, where the effects are seen on cells that have committed to the myeloid lineage and are responsive to the growth factors and cytokines used to assess megakaryocyte colony formation.

The presence of CD15+ cells indicates the availability of granulocytes. Compared to the controls, CD15+ cells do not appear to be affected over the dose range of the Homspera®, regardless of whether it was used alone or with the “priming” cocktail in the primary culture. There is some increase in expression above background levels with Homspera®+prime at 10⁻¹¹M where about 55% of CD45+ cells have the CD15+ marker—compared to controls at about 42%. However, the decrease in granulocytes is made up by the increase in CD14+ cells both with Homspera® alone and Homspera®+prime. Although an apparent outlier occurs at 0.1 pM for Homspera® alone and at 1 pM at Homspera®+prime, the CD14+ population increases. The effects are greatest with Homspera® alone where about 15% of CD45+ cells express CD14+; values that are above background CD14+ expression levels of about 11%. Thus, the increase in CD 14+ cells may be at the expense of the CD15+ granulocytes. Separate colony forming assays that examine stem cell differentiation have confirmed the ability of Homspera® to increase the numbers of granulocyte/macrophage colonies from bone marrow stem cell samples. However, the colony forming assay does not distinguish between granulocyte and macrophage precursor cells. The combined data suggests Homspera® may have more effect on early stage macrophage cells compared to granulocytes, and that Homspera® could be preferentially acting on the macrophage population as the quiescent cells enter the cell cycle. With these cells, Homspera® appears to function at both an early stage as the stem cells leave quiescence and at a later stage as the stem cells have committed to the myeloid lineage and begin differentiating.

Erythroid cells were examined using antibodies against Glycophorin-A+(CD235a) cells. Both Homspera® alone and Homspera®+prime produce an interesting, but almost identical response that appears to be out of phase by one log. The proportion of cells at the lowest Homspera® dose also appears to be similar to the controls, and the whole response does not significantly increase above the respective controls (0.75% for Homspera®+prime and about 1% for Homspera® alone). Despite the responses being close to control values, the shift in response to the left for the Homspera®+prime would imply that the cells might have become more sensitive. While Homspera® is not effective in this assay when given to the cells during a “priming” period, Homspera® does function to stimulate erythroid cells at a later stage as confirmed by colony forming assays. See, for example, Examples 4 and 6. In a colony forming assay, Homspera® enhances the formation of red blood cell precursors more than two-fold relative to controls lacking Homspera®. These differences support a model where Homspera® increases red blood cell precursors by acting on stem cells that have already entered the cell cycle and have committed to the myeloid lineage.

The presence of the CD38 expression marker is an indication of committed hematopoietic progenitors and some precursor cells like Blast Forming Unit-Erythroid, and Colony Forming Unit-Granulocyte/Macrophage. The response of CD38+ cells is, in many respects, similar to that of Glycophorin-A+ cells, in that the response to Homspera® alone and Homspera®+prime is similar, but out of phase by one log, with the Homspera®+prime cells indicating greater sensitivity-because maximal expression (about 35% of CD45+ cells) occurred at 10⁻¹²M Homspera®, compared to maximal stimulation of Homspera® alone at a Homspera® concentration of 10⁻¹¹M (about 35% of CD45+ cells). An increased proportion of CD38+ cells are also seen with Homspera®+prime in the controls, although as a percent of CD45+ cells, there is little difference. There is no dose dependent response of the CD117+ cell population to either Homspera® alone or Homspera®+prime, but Homspera®+prime stimulated the percentage of CD45+ cells expressing CD117 compared to Homspera® alone (about 2.5% to about less than 0.5%).

Antibodies against CD3+, CD19+, CD56+ cells were used to identify cells of the lymphoid lineage. For the Homspera®+prime treatment, there appears to be increase in CD3+ T lymphocytes reaching a maximum of 50% of CD45+ cells at 1 pM Homspera® which is above background values at approximately 35% of CD45+ cells. Although variations occur with Homspera® alone, most values are similar to those of the control. In contrast to the response with CD3+ cells, Homspera® alone is increased above control (1.1% of CD45+) for the CD19+ B-lymphocyte population with a maximum of about 5% CD45+ cells at 1 pM Homspera®. The situation appears to be similar for the CD56+ NK cell population, with Homspera® alone, although with respect to the controls, the situation is reversed and CD56 expression does not differ appreciably from controls either with Homspera® alone or Homspera®+prime.

C. Conclusions

The phenotypic response to Homspera® alone and Homspera®+prime are cell-type specific, indicating that for some populations, Homspera® alone has a dramatic effect, while for others Homspera®+prime has an effect. In addition, both treatments result in a response different from that of the controls. In contrast to the lack of response at the proliferation level, the response on expression markers at the stem cell level and on committed differentiating cells is quite dramatic.

Further, the data indicate that, at the stem cell level, a stimulation and/or potentiation occurs, which in turn, results in an increase or decrease in specific lineage populations, as confirmed by colony forming assays. Thus, the increase in CD90+ cells could increase the influx of lymphopoietic stems into the T- and B-lymphocyte lineages. An increase in hematopoietic stem cells expressing CD34+/CD133+ markers would indicate the increase in specific hematopoietic committed cells, especially megakaryocytes and CD14+ cells, the latter at the expense of the CD15+ granulocytes.

### Example 6

### An Exemplary Substance P Analog, Homspera®, Stimulates Proliferation of Exemplary Adult Stem Cells, Human Bone Marrow Cells (HBMCs)

The objective of this study was to compare stem cell hematopoiesis of Homspera® with native, C-terminally amidated substance P using human bone marrow cells in vitro.

A. Materials and Methods

Colony forming unit (CFU) assays were used to examine the effects of Homspera® in stimulating hematopoietic stem cells isolated from human bone marrow to differentiate into lineage-specific progenitor cells. As stem cells differentiate in response to growth factors, they form a colony of cells with distinct morphologies that can be visualized using a microscope. This study examined the formation of three different progenitor cell populations, erythrocytes, platelets and granulocytes/macrophages.

Bone marrow aspirates were obtained from three healthy donors between 18 and 35 years of age following appropriate guidelines and protocols. Bone marrow mononuclear cells were isolated using a Ficoll-Hypaque density gradient, separating red blood cells from the others. Cells from each donor were processed independently and used for setting up individual experiments to assess the effects, if any, of donor variability.

Cells (1×10⁵) were plated in duplicate onto 35 mm tissue culture plates for each condition tested and set up as described in Rameshwar et al. 1993, Blood. 81:2, 391-398. Erythroid and granulocyte/macrophage cultures were plated using methylcellulose and megakaryocyte cultures (platelet precursors) used a collagen-based support (StemCell Technologies, Vancouver, Canada, catalogue #04973). Cells were cultured in the presence of either Homspera® or substance P at various concentrations. In the platelet study, neurokinin receptor antagonists were used to demonstrate receptor-specific effects. CP 99,994 (Pfizer) was used as a neurokinin-1 receptor antagonist and SR 48968 (Sanofi) was used as a neurokinin-2 receptor antagonist.

The cytokines and growth factors added to Blast Forming Unit-Erythroid (BFU-E) cultures, Colony Forming Unit-Erythroid (CFU-E) cultures, and Colony Forming Unit-Granulocyte/Macrophage (CFU-GM) cultures were added as defined by proliferative units. BFU-E cultures contained 2 Units human interleukin-3 (hIL-3) and 2 Units recombinant human erythropoietin (rhEpo). Two Units IL-3 is about 0.1 ng/3 ml. Rameshwar, private correspondence. CFU-E cultures contained 2 Units rhEpo, and CFU-GM cultures contained 2.6 Units recombinant human granulocyte macrophage-colony stimulating factor (GM-CSF). The 2.6 Units of GM-CSF is about 2 ng/3 ml. Rameshwar, private correspondence.

The biological activity in proliferative units for Epo was characterized by R&D Systems, Inc. in a cell proliferation assay using TF-1 cells, a factor-dependent human erythroleukemic cell line. Kitamura, et al., 1989, J. Cell. Physiol. 140: 323-334. The units for hIL-3 and GM-CSF were defined using an IL-3/GM-CSF-dependent cell line, M-07e, a subline of the M-07 human megakaryoblastic leukemia cell line. Avanzi, G et al., 1990, J. Cell. Physiol., 145:458-464. Standard growth curves were established using serial dilutions of rhIL-3 (50 ng/ml) or rhGM-CSF (1 ng/ml). One cytokine proliferative unit was defined as the amount required to stimulate one-half maximal growth of the M-07e cells.

Cultures were set up with limited cytokines and growth factors which would promote differentiation of one progenitor per plate. Growth factors and cytokines added to Colony Forming Unit-Megakaryocyte (CFU-Mk) cultures used weight/volume ratios and included 50 ng/ml recombinant human thrombopoietin (rhTpo), 10 ng/ml recombinant human interleukin-6 (rhIL-6), and 10 ng/ml recombinant human interleukin-3 (rhIL-3) as suggested and supplied by StemCell Technologies.

Cells were cultured in the presence of Homspera® or substance P over the following molar concentrations: 10⁻⁷, 10⁻⁸, 10⁻⁹, 10⁻¹⁰, 10⁻¹¹, 10⁻¹², 10⁻¹³, and 10⁻¹⁴M. Each compound was dissolved in endotoxin-free water to a final concentration of 0.1 mM. After reconstitution, the solutions were aliquoted into siliconized microcentrifuge tubes and exposed to nitrogen gas, eliminating oxygen in the head space of the enclosure. All aliquots were stored at −20° C. until use and used within one month of reconstitution. See, Rameshwar, et al. 1997, J. Immunol. 158:3417-3424.

Control plates without Homspera® or substance P were used to assess the baseline for colony growth. Cultures were incubated at 37° C. with 5% CO₂ for approximately 14 days, after which colonies were manually enumerated using a microscope. To count megakaryocyte colonies, the cells were fixed followed by a staining procedure using the following antibodies: primary—mouse anti-human GP11b/111a, isotype control—mouse anti-trinitrophenyl, secondary—biotin-conjugated goat anti-mouse IgG, detection—avidin-alkaline phosphatase conjugate.

The results are expressed as percent colonies of control cultures (without Homspera® or substance P). The number of control colonies were normalized to 100% and represented as a zero level on the Y-axis.

B. Results

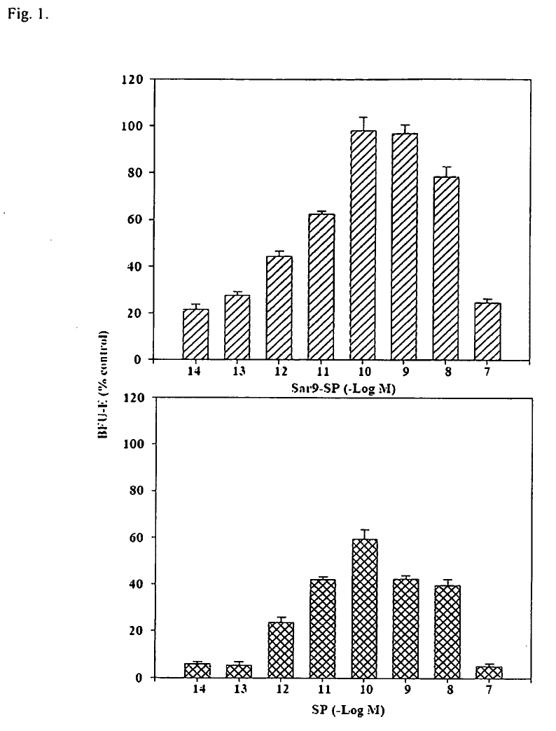

Homspera® was more effective than substance P at increasing colony counts for all stem cell progenitors examined. Two different red blood cell progenitor types were examined, BFU-E and CFU-E, which derive from a common progenitor, colony forming unit-granulocyte erythrocyte macrophage megakaryocyte (CFU-GEMM). BFU-E mature into CFU-E, which ultimately develop into functional red blood cells. (FIGS. 1 & 2)

Homspera® was more effective than substance P at enhancing stem cell differentiation. Homspera® enhanced BFU-E colony formation 2-fold (or 100%) relative to controls, whereas substance P increased colonies about 1.5-fold (or 60%) from control values (FIG. 1). These effects are similar to those of Example 4.

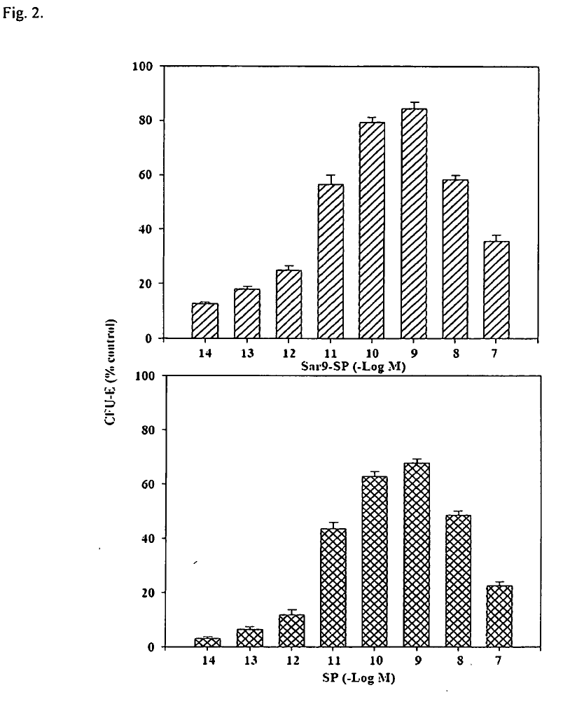

For CFU-E, the difference between Homspera® and substance P treatment was less pronounced. For example, Homspera® enhanced colony formation to greater than 80% from control values, while substance P enhanced colony formation to about 70% from control values (FIG. 2).

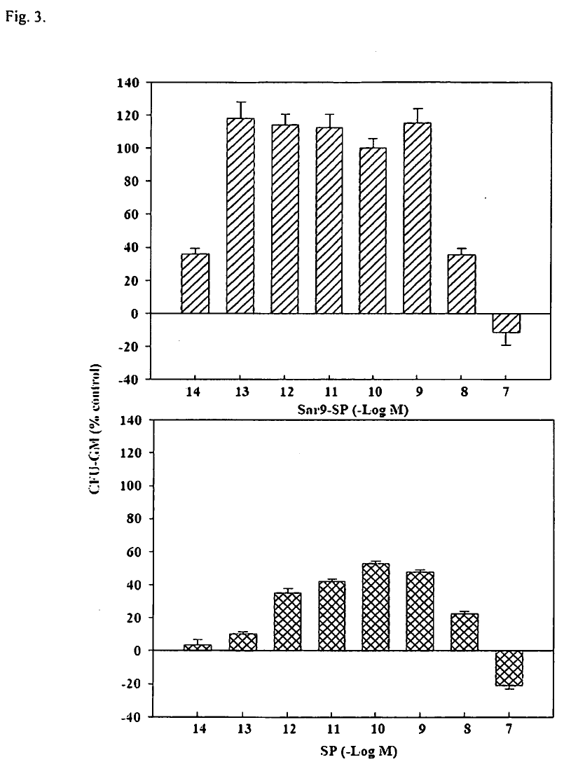

This study demonstrated greater than a 2-fold increase in granulocyte/macrophage precursors when cultured with several different Homspera® concentrations from 10⁻¹³ to 10⁻⁹M (FIG. 3), and Homspera® was twice as effective as substance P at stimulating differentiation of human stem cells into CFU-GM. These results suggest that Homspera® could increase circulating levels of granulocytes and macrophages in vivo, possibly acting to mobilize the progenitors from the bone marrow or through a combination of differentiation and mobilization. The two-fold stimulation is similar to levels observed in Example 4.

Homspera® and substance P treatments each demonstrated approximately a 2-fold stimulatory effect above controls for platelet precursors. However, Homspera® was effective at a concentration one log unit below substance P (10⁻⁹M vs. 10⁻⁸M). To demonstrate that the effects of substance P were occurring through activation of the neurokinin-1 receptor, two different receptor antagonists were used in the presence of substance P. CP-99,994 is a neurokinin-1 receptor antagonist which blocks the stimulatory activity of substance P. Additionally, a neurokinin-2 receptor antagonist SR48968 was used, which showed no effect on substance P activity in enhancing platelet colony formation indicating that the effects of substance P on Mk colonies are via the neurokinin-1 receptor. The effects of Homspera® on platelet precursors is similar to the two-fold levels observed in Example 4.

C. Conclusion

These data indicate Homspera® can stimulate hematopoiesis of all 3 major blood cell types. Homspera® was effective at concentrations ranging from 10⁻⁷M to 10⁻¹⁴M. In addition, Homspera® was more potent than substance P in enhancing colony formation of BFU-E, CFU-E, CFU-GM and CFU-Mk.

### Example 7

### The Effect of an Exemplary Substance P Analog, Homspera® on Fibroblast Proliferation

A. Material and Methods

Homspera® (as the acetate salt) was obtained by ImmuneRegen from CS Bio. The peptide was shipped under refrigerated conditions and stored at −20° C. until reconstitution. Reconstitution of Homspera® was performed by dissolving compound to 1 mg/ml final concentration in sterile phosphate buffer saline (PBS) pH 7.4, then storing reconstituted Homspera® at 4° C. in polypropylene enclosure. Appropriate dilutions were made from this 1 mg/ml working stock by diluting with sterile PBS. Spantide I (CAS 91224-37-2) was obtained from Sigma Aldrich and was added at a concentration of 10 μM. Normal human fibroblasts were obtained from ATCC (passage 2-3) and grown in IMDM-Glutamax media (Invitrogen #31980-030) containing 10% Fetal Bovine Serum (FBS) (Invitrogen #10437-028) and penicillin-streptomycin-amphotericin B (Invitrogen #15240-104). These cells were cultivated up to passage 40. Cells were trypsinized using 0.05% Trypsin (Invitrogen #15400-054) in calcium and magnesium-free Hanks solution (Invitrogen #14170), followed by neutralization in Iscoves medium containing 10% FBS. Cells were maintained in a cell incubator at 37° C. and 5% CO₂.

To quantify proliferation, MTT assay (Invitrogen Molecular Probes M6494) was performed. See, Mosman, 1983, J. Immunol. Methods 65: 55. Briefly, cells were plated into 96-well tissue culture plates at 2,000 cells per well. Cultures were then treated with Homspera®; total well volume was kept to 0.1 mL. MTT was weighed (5 mg) and dissolved in distilled water, filtered using a 200 micron syringe filter and stored in the dark at 4° C. Ten μL MTT was added to each well and mixed. Cultures were incubated for 4 hours with MTT. Then medium was removed and 200 μL DMSO was added to each well and the absorbance was measured on an ELISA plate reader with a test wavelength of 570 nm and a reference wavelength of 630 nm to obtain sample signal.

To test whether Homspera® may induce proliferation directly or acts to facilitate growth-factor driven proliferation, Homspera® was tested at various concentrations (within the range of 0.01-10 μM) under varying growth conditions (serum-free or serum containing) for defined periods of time (24, 48 or 72 hrs) under serum-starved (0.5% FBS) or serum containing conditions (2.5% vs. 5% vs. 10% FBS), respectively. To determine the optimal concentration of serum, MTT assays were performed on fibroblasts grown in media containing 0.5%, 2.5%, 5% or 10% FBS. These studies showed that normal foreskin fibroblasts survive in serum-starved conditions (containing 0.5% FBS) for 5 days and can be propagated in media containing 5% FBS while achieving maximal growth in 4 days. In each of these experiments, the mean optical density (OD) was representative of eight replicates.

To establish the relationship between the number of viable cells and OD (derived from the MTT assay), cells were seeded in various amounts (2,000-200,000 cells/well) and subjected to an MTT assay. Mean OD was calculated from eight replicates. Graphical analysis (OD vs. number of viable cells) revealed that saturation occurs between 0.7-0.8 OD units at a cell density between 30,000-40,000 cells/well. Growth curves generated by seeding 2000 cells/well indicate this occurs within 4-5 days of growth. Hence, all further experiments were performed within this time frame, so as to expose cells to Homspera® within their proliferative phase of growth.

One group of 10 μM Homspera®-treated fibroblasts, was also exposed to 10 μM Spantide I, a neurokinin-1 receptor antagonist. See, Hazlett et al., 2007, Investigative Opthalmology Visual Sci. 48: 797-807.

Experiments were conducted with Homspera® as follows: normal foreskin fibroblasts were seeded in a 96 well plate using IMDM (media) containing 0.5% FBS. The following day, these serum-starved cells were treated with various amounts of Homspera® or Homspera®+antagonist-(Spantide I) for a period of 1 or 3 days. Cells were pretreated with Homspera® in serum free media (0.5% FBS) for 3 hours. Spantide I was added 1-hour prior to the addition of Homspera® in the 10 μM-Homspera®-treated group treated with Spantide I. Cells were then re-stimulated with serum (5% FBS) or not (0.5% FBS) while maintaining the presence of Homspera® and/or Spantide I. MTT assays were performed after 1 and 3 days of treatment with Homspera®. Three independent experiments (n=3) were performed and mean OD was represented as average of eight replicates. One experiment had a lower starting number of cells and was excluded from the analysis. Hence, the following analysis is representative of two independent experiments (n=2) done in replicates of eight. Results are represented as % growth where mean OD of each sample is normalized to its control (Table 9).

B. Results

In cultures exposed to 5% FBS, Homspera® trends toward increasing proliferation after 1 day of exposure. Peak percentage growth (almost 143%) was seen at 10 μM Homspera®, with 1 μM Homspera® providing proliferation about 135% of control. At concentrations of 0.1 μM and less, growth was increased to about 115% of control (114.8% at 0.01 μM Homspera® concentration and 115.5% at 0.1 μM Homspera® concentration). Homspera® increased proliferation at 1-day post-treatment in a dose-dependent manner when cultures were exposed to 5% FBS.

In cultures exposed to 0.5% FBS, treatment with increasing concentrations of Homspera® trends toward increasing proliferation after 3 days of treatment. No effect on proliferation was observed at 1-day post-exposure for cultures exposed to 0.5% FBS.

TABLE 9
Percentage growth of fibroblasts normalized to control.

| | | 0.5% FBS | | 5% FBS | |
|---|---|---|---|---|---|
| | Days of | % | Std | % | Std |
| Sample | treatment | growth | dev | growth | dev |
| Vehicle control | 1 day | 100.0 | 0.0 | 100.0 | 0.0 |
| 0.01 μM Homspera ® | | 86.1 | 12.8 | 114.8 | 3.6¹ |
| 0.1 μM Homspera ® | | 86.0 | 3.4 | 115.5 | 10.9² |
| 1 μM Homspera ® | | 85.0 | 5.9 | 134.7 | 19.9³ |
| 10 μM Homspera ® | | 82.2 | 8.8 | 142.8 | 34.4⁴ |
| 1 μM Homspera ® + | | 79.4 | 12.9 | 120.4 | 20.6⁵ |
| 10 μM Spantide I | | | | | |
| Vehicle control | 3 days | 100.0 | 0.0 | 100.0 | 0.0 |
| 0.01 μM Homspera ® | | 107.4 | 6.3 | 106.4 | 6.8 |
| 0.1 μM Homspera ® | | 103.4 | 10.1 | 105.4 | 3.2 |
| 1 μM Homspera ® | | 106.7 | 4.6 | 102.6 | 0.6 |
| 10 μM Homspera ® | | 118.3 | 7.8 | 97.3 | 1.5 |
| 1 μM Homspera ® + | | 114.5 | 2.6 | 87.2 | 6.1 |
| 10 μM Spantide | | | | | |

¹1-tail P-value compared to vehicle control = 0.177497
²1-tail P-value compared to vehicle control = 0.261254
³1-tail P-value compared to vehicle control = 0.028291
⁴1-tail P-value compared to vehicle control = 0.118126
⁵1-tail P-value compared to vehicle control = 0.08662

C. Conclusion

The effects of Homspera® on human dermal fibroblasts were evaluated in both “serum-starved” (0.5% FBS) and serum-exposed (5% FBS) conditions. An increasing proliferative effect was observed in cultures treated with Homspera® and exposed to 5% FBS at 1-day post-treatment. At 1-day post treatment, cultures exposed to 1 μM Homspera® and 5% FBS had statistically significant (P<0.05) increases in proliferation as determined by MTT assay. Other groups in this series (5% FBS at 1 day of exposure) exhibited an increase in proliferation as well (Table 1). This proliferative effect was less pronounced in cultures exposed to 5% FBS and treated with Homspera® for 3 days. An increasing proliferative effect was also observed in cultures treated with Homspera® and exposed to serum-started (0.5% FBS) conditions at 3-days post-treatment. Thus, Homspera® could have a short-term (about 1-day) effect on the proliferation of human dermal fibroblasts cultured in 5% FBS and a longer-term (about 3 day) effect on the proliferation of human dermal fibroblasts cultured in 0.5% FBS as determined by MTT assay.

### Example 8

### Use of an Exemplary Substance P Analog, Homspera®, to Promote Wound Healing

The objective of this study was to determine the efficacy of Homspera® (Sar⁹, Met (O₂)¹¹-substance P) following topical administration in a 28-day deep wound healing Yorkshire pig model.

A. Materials & Methods

Homspera® (Lot #E844) was formulated in PBS as follows. Three mg of Homspera® were slowly added to 18 mL of sterile Dulbecco's Phosphate Buffered Saline (DPBS) (Mediatech, Cat#21-031-CV, Lot#21031267) while stirring at room temperature. The stirring was continued till the solution was clear, making a stock solution of 10⁻⁴M. From this stock, solution with various concentrations of 10⁻⁶M, 10⁻⁸M, 10⁻¹⁰M were prepared using DPBS as the diluents.

Two (2) normal, female Yorkshire pigs three months in age, weighing 25-75 kg, were quarantined and acclimated in-house for at least 6 days. Animals were identified using ear tattoos. Animals were housed in pens; housing and sanitation were performed according to standard operating procedures. Animals were provided a laboratory canine diet, and were provided tap water ad libitum.

Food was withheld from animals for at least 12 hours prior to administration of anesthesia. Anesthetization was done by intramuscular injection of 20 mg/kg ketamine and 2 mg/kg Xylazine. On the day of wounding (day 0), the pigs were anesthetized. This was followed by intubation and inhalation of 1-2% Isoflurane. The dorsal and lateral thorax and abdomen of the pig were clipped with a #40 Oster clipper blade and washed with an antibiotic-free soap. A total of 20 full-thickness wounds each with a 3 cm diameter were created on each pig using a scalper, 2 cm apart and 10 per side of the animal. Tattoo labels were made around each wound for measuring purposes. Pigs were observed daily for morbidity and toxic signs for 21 days after wound induction. Any signs of clinical illness, such as fever, decreased appetite, reluctance to move, diarrhea, dehydration, infection, etc. were treated per the veterinarian's instruction.

Homspera® (at 10⁻⁴M, 10⁻⁶M, 10⁻⁸M and 10⁻¹⁰M) and control (DPBS) articles were applied topically intra-wound. On each pig, 2 ml of Homspera® at the test concentration or control solution was applied to fill the wound and covered it with saline-moistened (not wet) non-adherent Telfa gauze. The gauze was secured using Transpore tape. All wounds were covered with a blue pad, absorbent layer against the skin. Both pigs were wrapped with a layer of elastic bandaging to prevent movement of the dressings. Sterile techniques were performed as much as possible during the surgery to minimize the risk of infection on wound area. Dressings were changed every 5 days and Homspera® was re-applied at each dressing change.

Wound healing was evaluated by measuring the diameters of wound closure in 4 different directions on designated time points at Days 7, 10, 14, 17, 21, and 24 post-wounding.

B. Results

Wound measurements of each wound were taken at Days 7, 10, 14, 17, 21, and 24 post-wounding. There was a trend of reduced wound area for all Homspera® treatment groups. The animals that received the highest dose of Homspera® (10⁻⁴M) responded the best, showing a nearly 27% reduction in wound size (area) at 14 days post-wounding compared to vehicle-treated controls and a 37% reduction in wound area at Day 21 post-wounding compared to vehicle-treated controls. A reduction in wound size was observed over the time period of Days 7-24 compared to controls. Other doses of Homspera® accelerated the closure of wounds, and the highest dose exhibited the most profound effect.

C. Conclusion

Homspera® treatment reduced wound areas at all time points between days 7 and 24 post-wounding. The highest dose of Homspera® treatment tested, 10⁻⁴M, was most efficacious, yielding a reduction in wound areas of 27% compared to control at Day 14 and a reduction of 37% compared to control at Day 21.

While the methods and compositions have been described with respect to specific examples including presently preferred modes of carrying out certain embodiments, those skilled in the art will appreciate that there are numerous variations and permutations of the above described systems and techniques that fall within the spirit and scope of the invention as set forth in the appended claims.

All references cited herein are incorporated herein by reference in their entireties for all purposes.

What is claimed is:

1. A method of treating or ameliorating a stem cell disorder comprising administering to a subject an effective amount of a substance P analog; wherein the substance P analog is of Formula (I):
(SEQ ID NO: 11)
Z₁-Xaa¹-Xaa²-Xaa³-Xaa⁴-Xaa⁵-Xaa⁶-Xaa⁷-Xaa⁸-Xaa⁹-
Xaa¹⁰-Xaa¹¹-Z₂ (I)
or a pharmaceutically acceptable salt thereof, wherein:
Xaa¹ is Arg, Lys, 6-N methyllysine or (6-N,6-N)dimethyllysine;
Xaa² is Pro or Ala;
Xaa³ is Lys, Arg, 6-N-methyllysine or (6-N,6-N)dimethyllysine;
Xaa⁴ is Pro or Ala;
Xaa⁵ is Gln or Asn;
Xaa⁶ is Gln or Asn;
Xaa⁷ is Tyr, Phe or Phe substituted with chlorine at position 2, 3 or 4;
Xaa⁸ is Tyr, Phe, or Phe substituted with chlorine at position 2, 3 or 4;
Xaa⁹ is Gly, Pro, Ala or N-methylglycine;
Xaa¹⁰ is Leu, Val, Ile, Norleucine, Met, Met sulfoxide, Met sulfone, N-methylleucine, or N-methylvaline;
Xaa¹¹ is Met, Met sulfoxide, Met sulfone, or Norleucine;
Z₁ is R₂N— or RC(O)NR—;
Z₂ is —C(O)NR₂ or —C(O)OR or a salt thereof;
each R is independently —H, (C₁-C₆) alkyl, (C₁-C₆) alkenyl, (C₁-C₆) alkynyl, (C₅-C₂₀) aryl, (C₆-C₂₆) alkaryl, 5-20 membered heteroaryl or 6-26 membered alkheteroaryl; and
each “-” between residues Xaa¹ through Xaa¹¹ independently designates an amide linkage, a substitute amide linkage or an isostere of an amide.

2. The method of claim 1, wherein,
Xaa¹ is Arg;
Xaa² is Pro;
Xaa³ is Lys;
Xaa⁴ is Pro;
Xaa⁵ is Gln;
Xaa⁶ is Gln;
Xaa⁷ is Tyr, Phe or Phe substituted with chlorine at position 4;
Xaa⁸ is Tyr, Phe, or Phe substituted with chlorine at position 4;
Xaa⁹ is Gly, Pro or N-methylglycine;
Xaa¹⁰ is Leu; and
Xaa¹¹ is Met, Met sulfoxide, Met sulfone or Norleucine.

3. The method of claim 1, wherein the “-” between residues Xaa¹ through Xaa¹¹ designates —C(O)NH—;
Z₁ is H₂N—; and
Z₂ is —C(O)NH₂.

4. The method of claim 1, wherein the substance P analog is:

| | |
|---|---|
| RPKPQQFFGLM; | (SEQ ID NO: 1) |
| RPKPQQFFGLNle; | (SEQ ID NO: 2) |
| RPKPQQFFPLM; | (SEQ ID NO: 3) |
| RPKPQQFFMeGlyLM; | (SEQ ID NO: 4) |
| RPKPQQFTGLM; | (SEQ ID NO: 5) |
| RPKPQQF(4-Cl)F(4-Cl)GLM; | (SEQ ID NO: 6) |
| RPKPQQFFGLM(O); | (SEQ ID NO: 7) |
| RPKPQQFFMeGlyLM(O); | (SEQ ID NO: 8) |
| RPKPQQFFGLM(O₂); | (SEQ ID NO: 9) |
| or | |
| RPKPQQFFMeGlyLM(O₂). | (SEQ ID NO: 10) |

5. The method of claim 1, wherein the substance P analog is
Z₁—RPKPQQFFMeGlyLM(O₂)—Z₂;
wherein Z₁ is NH₂ and Z₂ is C(O)NH₂.

6. The method of claim 1, wherein the stem cell disorder is amegakaryocytosis, aplastic anemia, blackfan-diamond anemia, congenital cytopenia, congenital dyserythropoietic anemia, dyskeratosis congenital, Fanconi anemia, paroxysmal nocturnal hemoglobinuria (PNH), pure red cell aplasia, acute myelofibrosis, agnogenic myeloid metaplasia, polycythemia vera, essential thrombocythemia, beta thalassemia major, sickle cell disease, familial erythrophagocytic lymphohistiocytosis, hemophagocytosis, Langerhans' cell histiocytosis (hystiocytosis X), chronic granulomatous disease, congenital neutropenia, ataxia-telangiectasia, myelokathexis, bare lymphocyte syndrome, leukocyte adhesion deficiency, severe combined immunodeficiencies (SCID), common variable immunodeficiency, bare lymphocyte syndrome, Chediak-Higashi syndrome, Kostmann syndrome, Omenn syndrome, purine nucleoside phosphorylase deficiency, reticular dysgenesis, Wiskott-Aldrich syndrome, X-linked lymphoproliferative disorder, adrenoleukodystrophy fucosidosis, Gaucher disease, Hunter's syndrome (MPS-II), Hurler's syndrome (MPS-IH), Krabbe disease, Lesch-Nyhan syndrome, mannosidosis, Maroteaux-Lamy syndrome (MPS-VI), metachromatic leukodystrophy, mucolipidosis II (I-cell disease), neuronal ceroid lipofuscinosis (Batten disease), Niemann-Pick disease, Sandhoff disease, San Filippo syndrome (MPS-III), Morquio Syndrome (MPS-IV), Sly Syndrome, Beta-Glucuronidase deficiency (MPS-VII), andrenoleukodystrophy, Scheie syndrome (MPS-IS), sly syndrome, Tay Sachs, Wolman disease, Mucopolysaccharidoses (MPS), acute biphenotypic leukemia, acute lymphocytic leukemia (ALL), acute myelogenous leukemia (AML), acute undifferentiated leukemia, adult T cell leukemia, adult T cell lymphoma, chronic lymphocytic leukemia (CLL), chronic myelogenous leukemia (CML), Hodgkin's lymphoma, juvenile chronic myelogenous leukemia (JCML), juvenile myelomonocytic leukemia (JMML), myeloid/natural killer cell precursor acute leukemia, non-Hodgkin's lymphoma, polymphocytic leukemia, acute myelofibrosis, agnogenic myeloid metaplasia (myelofibrosis), amyloidosis, chronic myelomonocytic leukemia (CMML), essential thrombocythemia, polycythemia vera, multiple myeloma, plasma cell leukemia, Waldenstrom's macroglobulinemia, cartilage-hair hypoplasia, Glanzmann thrombasthenia, amegakaryocytosis, congenital thrombocytopenia, congenital erythropoietic porphyria (Gunther disease), DiGeorge syndrome, osteopetrosis, brain tumors, Ewing sarcoma, neuroblastoma, ovarian cancer, breast cancer, neuroblastoma, renal cell carcinoma, rhabodomyosarcoma, small cell lung cancer, testicular cancer, thymoma (thymic carcinoma), chronic active Epstein barr, Evans syndrome, multiple sclerosis, rheumatoid arthritis, systemic lupus erythematosus, thymic dysplasia, Chediak-Higashi syndrome, chronic granulomatous disease, neutrophil actin deficiency, reticular dysgenesis, deafness, loss of hearing, diabetes, heart disease, liver disease, muscular dystrophy, Parkinson's disease, spinal cord injury or stroke.

7. The method of claim 1, wherein leukocytes, lymphocytes, neutrophils, band cells, monocytes, granulocytes, erythrocytes, eosinophils, basophils or platelets are increased in the subject.

8. The method of claim 7 wherein the lymphocytes are T lymphocytes or B lymphocytes.

9. The method of claim 1 wherein administration of the substance P analog results in increased differentiation of high proliferative potential-stem and progenitor cells (HPP-SP cells), colony forming cells-granulocyte, erythroid, macrophage, megakaryocyte cells, (CFC-GEMM cells), granulocyte-macrophage-colony forming cells (GM-CFC), megakaryocyte-colony forming cells (Mk-CFC), T-lymphocyte-colony forming cells (T-CFC), B-lymphocyte-colony forming cells (B-CFC), colony forming unit-megakaryocyte cells (CFU-Mk cells), blast forming unit-erythroid cells (BFU-E cells), colony forming unit-erythroid cells (CFU-E cells), or colony forming unit-granulocyte/macrophage cells (CFU-GM cells).

10. The method of claim 9 wherein the subject is human.

11. A composition comprising a cell and a substance P analog in an amount effective to stimulate differentiation of the cell wherein the substance P analog is of Formula (I):
(SEQ ID NO: 11)
Z₁-Xaa¹-Xaa²-Xaa³-Xaa⁴-Xaa⁵-Xaa⁶-Xaa⁷-Xaa⁸-Xaa⁹-
Xaa¹⁰-Xaa¹¹-Z₂ (I)
or a pharmaceutically acceptable salt thereof, wherein:
Xaa¹ is Arg, Lys, 6-N methyllysine or (6-N,6-N)dimethyllysine;
Xaa² is Pro or Ala;
Xaa³ is Lys, Arg, 6-N-methyllysine or (6-N,6-N)dimethyllysine;
Xaa⁴ is Pro or Ala;
Xaa⁵ is Gln or Asn;
Xaa⁶ is Gln or Asn;
Xaa⁷ is Tyr, Phe or Phe substituted with chlorine at position 2, 3 or 4;
Xaa⁸ is Tyr, Phe, or Phe substituted with chlorine at position 2, 3 or 4;
Xaa⁹ is Gly, Pro, Ala or N-methylglycine;
Xaa¹⁰ is Leu, Val, Ile, Norleucine, Met, Met sulfoxide, Met sulfone, N-methylleucine, or N-methylvaline;
Xaa¹¹ is Met, Met sulfoxide, Met sulfone, or Norleucine;
Z₁ is R₂N— or RC(O)NR—;
Z₂ is —C(O)NR₂ or —C(O)OR or a salt thereof;
each R is independently —H, (C₁-C₆) alkyl, (C₁-C₆) alkenyl, (C₁-C₆) alkynyl, (C₅-C₂₀) aryl, (C₆-C₂₆) alkaryl, 5-20 membered heteroaryl or 6-26 membered alkheteroaryl; and
each “-” between residues Xaa¹ through Xaa¹¹ independently designates an amide linkage, a substitute amide linkage or an isostere of an amide.

12. The composition of claim 11, wherein,
Xaa¹ is Arg;
Xaa² is Pro;
Xaa³ is Lys;
Xaa⁴ is Pro;
Xaa⁵ is Gln;
Xaa⁶ is Gln;
Xaa⁷ is Tyr, Phe or Phe substituted with chlorine at position 4;
Xaa⁸ is Tyr, Phe, or Phe substituted with chlorine at position 4;
Xaa⁹ is Gly, Pro or N-methylglycine;
Xaa¹⁰ is Leu; and
Xaa¹¹ is Met, Met sulfoxide, Met sulfone or Norleucine.

13. The composition of claim 11, wherein the “-” between residues Xaa¹ through Xaa¹¹ designates —C(O)NH—;
Z₁ is H₂N—; and
Z₂ is —C(O)NH₂.

14. The composition of claim 11, wherein the substance P analog is:

| | |
|---|---|
| RPKPQQFFGLM; | (SEQ ID NO: 1) |
| RPKPQQFFGLNle; | (SEQ ID NO: 2) |
| RPKPQQFFPLM; | (SEQ ID NO: 3) |
| RPKPQQFFMeGlyLM; | (SEQ ID NO: 4) |
| RPKPQQFTGLM; | (SEQ ID NO: 5) |
| RPKPQQF(4-Cl)F(4-Cl)GLM; | (SEQ ID NO: 6) |
| RPKPQQFFGLM(O); | (SEQ ID NO: 7) |
| RPKPQQFFMeGlyLM(O); | (SEQ ID NO: 8) |
| RPKPQQFFGLM(O₂); | (SEQ ID NO: 9) |
| or | |
| RPKPQQFFMeGlyLM(O₂). | (SEQ ID NO: 10) |

15. The composition of claim 11, wherein the substance P analog is
Z₁—RPKPQQFFMeGlyLM(O₂)—Z₂;
wherein Z₁ is NH₂ and Z₂ is C(O)NH₂.

16. The composition of claim 11, wherein the cell is an undifferentiated cell.

17. The composition of claim 11, wherein the cell is a stem cell, a progenitor cell, or a partially differentiated cell.

18. The composition of claim 17, wherein the stem cell is a hematopoietic stem cell, lymphopoietic stem cell or myelopoietic stem cell.

19. The composition of claim 11, wherein the differentiation results in an increase in cells expressing CD15.

20. The composition of claim 11, wherein the substance P analog is administered parenterally.

21. A composition for promoting wound healing comprising cells, a matrix, and a substance P analog wherein the substance P analog is of Formula (I):
(SEQ ID NO: 11)
Z₁-Xaa¹-Xaa²-Xaa³-Xaa⁴-Xaa⁵-Xaa⁶-Xaa⁷-Xaa⁸-Xaa⁹-
Xaa¹⁰-Xaa¹¹-Z₂ (I)
or a pharmaceutically acceptable salt thereof, wherein:
Xaa¹ is Arg, Lys, 6-N methyllysine or (6-N,6-N)dimethyllysine;
Xaa² is Pro or Ala;
Xaa³ is Lys, Arg, 6-N-methyllysine or (6-N,6-N)dimethyllysine;
Xaa⁴ is Pro or Ala;
Xaa⁵ is Gln or Asn;
Xaa⁶ is Gln or Asn;
Xaa⁷ is Tyr, Phe or Phe substituted with chlorine at position 2, 3 or 4;
Xaa⁸ is Tyr, Phe, or Phe substituted with chlorine at position 2, 3 or 4;
Xaa⁹ is Gly, Pro, Ala or N-methylglycine;
Xaa¹⁰ is Leu, Val, Ile, Norleucine, Met, Met sulfoxide, Met sulfone, N-methylleucine, or N-methylvaline;
Xaa¹¹ is Met, Met sulfoxide, Met sulfone, or Norleucine;
Z₁ is R₂N— or RC(O)NR—;
Z₂ is —C(O)NR₂ or —C(O)OR or a salt thereof;
each R is independently —H, (C₁-C₆) alkyl, (C₁-C₆) alkenyl, (C₁-C₆) alkynyl, (C₅-C₂₀) aryl, (C₆-C₂₆) alkaryl, 5-20 membered heteroaryl or 6-26 membered alkheteroaryl; and
each “-” between residues Xaa¹ through Xaa¹¹ independently designates an amide linkage, a substitute amide linkage or an isostere of an amide.

22. The composition of claim 21, wherein,
Xaa¹ is Arg;
Xaa² is Pro;
Xaa³ is Lys;
Xaa⁴ is Pro;
Xaa⁵ is Gln;
Xaa⁶ is Gln;
Xaa⁷ is Tyr, Phe or Phe substituted with chlorine at position 4;
Xaa⁸ is Tyr, Phe, or Phe substituted with chlorine at position 4;
Xaa⁹ is Gly, Pro or N-methylglycine;
Xaa¹⁰ is Leu; and
Xaa¹¹ is Met, Met sulfoxide, Met sulfone or Norleucine.

23. The composition of claim 21, wherein the “-” between residues Xaa¹ through Xaa¹¹ designates —C(O)NH—;
Z₁ is H₂N—; and
Z₂ is —C(O)NH₂.

24. The composition of claim 21, wherein the substance P analog is:

| | |
|---|---|
| RPKPQQFFGLM; | (SEQ ID NO: 1) |
| RPKPQQFFGLNle; | (SEQ ID NO: 2) |
| RPKPQQFFPLM; | (SEQ ID NO: 3) |
| RPKPQQFFMeGlyLM; | (SEQ ID NO: 4) |
| RPKPQQFTGLM; | (SEQ ID NO: 5) |
| RPKPQQF(4-Cl)F(4-Cl)GLM; | (SEQ ID NO: 6) |
| RPKPQQFFGLM(O); | (SEQ ID NO: 7) |
| RPKPQQFFMeGlyLM(O); | (SEQ ID NO: 8) |
| RPKPQQFFGLM(O₂); | (SEQ ID NO: 9) |
| or | |
| RPKPQQFFMeGlyLM(O₂). | (SEQ ID NO: 10) |

25. The composition of claim 21, wherein the substance P analog is
Z₁—RPKPQQFFMeGlyLM(O₂)—Z₂;
wherein Z₁ is NH₂ and Z₂ is C(O)NH₂.

26. The composition of claim 21, wherein the cells are selected from the group consisting of: stem cells, progenitor cells, and fibroblasts.

27. The composition of claim 26 wherein the progenitor cells are endovascular progenitor cells or endothelial progenitor cells.

28. The composition of claim 21 wherein the cells are placental cells or umbilical cord blood cells.

29. The composition of claim 21, wherein the matrix is comprised of collagen, fibrinogen, fibrin, hydrogen or amniotic membrane allograft.

30. The composition of claim 21 wherein the wound is a diabetic wound.

31. The composition of claim 21 wherein the wound is a decubitus ulcer.


SEQUENCE LISTING (13 sequences)

SEQ ID NO: 1 - protein, 11 aa, Artificial Sequence
  Description of Artificial Sequence Synthetic peptide
RPKPQQFFGLM

SEQ ID NO: 2 - protein, 11 aa, Artificial Sequence
  Description of Artificial Sequence Synthetic peptide
RPKPQQFFGLX

SEQ ID NO: 3 - protein, 11 aa, Artificial Sequence
  Description of Artificial Sequence Synthetic peptide
RPKPQQFFPLM

SEQ ID NO: 4 - protein, 11 aa, Artificial Sequence
  Description of Artificial Sequence Synthetic peptide
RPKPQQFFXLM

SEQ ID NO: 5 - protein, 11 aa, Artificial Sequence
  Description of Artificial Sequence Synthetic peptide
RPKPQQFTGLM

SEQ ID NO: 6 - protein, 11 aa, Artificial Sequence
  Description of Artificial Sequence Synthetic peptide
RPKPQQXXGLM

SEQ ID NO: 7 - protein, 11 aa, Artificial Sequence
  Description of Artificial Sequence Synthetic peptide
RPKPQQFFGLX

SEQ ID NO: 8 - protein, 11 aa, Artificial Sequence
  Description of Artificial Sequence Synthetic peptide
RPKPQQFFXLX

SEQ ID NO: 9 - protein, 11 aa, Artificial Sequence
  Description of Artificial Sequence Synthetic peptide
RPKPQQFFGLX

SEQ ID NO: 10 - protein, 11 aa, Artificial Sequence
  Description of Artificial Sequence Synthetic peptide
RPKPQQFFXLX

SEQ ID NO: 11 - protein, 11 aa, Artificial Sequence
  Description of Artificial Sequence Synthetic peptide
XXXXXXXXXXX

SEQ ID NO: 12 - protein, 4 aa, Artificial Sequence
  Description of Artificial Sequence Synthetic peptide
RPKP

SEQ ID NO: 13 - protein, 11 aa, Artificial Sequence
  Description of Artificial Sequence Synthetic peptide
RPKPQQXXXLX